(19) United States
(12) Patent Application Publication
(10) Pub. No.: US 2009/0280103 A1
(43) Pub. Date: Nov. 12, 2009

(54) REGULATION OF MUSCLE REPAIR

(76) Inventor: Martin Flueck, Cheshire (GB)

Correspondence Address: GRANT ANDERSON LLP;C/O PORTFOLIOIP, P.O. BOX 52050, MINNEAPOLIS, MN, 55402, US

(21) Appl. No.: 12/415,988

(22) Filed: Mar. 31, 2009

Related U.S. Application Data
(60) Provisional application No. 61/042,593, filed on Apr. 4, 2008.
(60) Provisional application No. 61/075,239, filed on Jun. 24, 2008.

(51) Int. Cl. A61K 38/43; A61K 38/16; A61K 31/7088

(52) U.S. Cl. 424/94.5; 514/12; 514/44 R

(57) ABSTRACT

Biochemical signals originating at sites of focal adhesions between muscle fibers are functionally involved in the mechano-dependent governance of muscle gene expression. Herein included information describes a methodology to promote improvements of motor function by combining contraction-related and pharmacological interventions which stimulate focal adhesion signaling and a diagnostic use to identify the responsiveness to treatment of a subject.

### RELATED PATENT APPLICATIONS

This patent application claims the benefit of U.S. provisional patent application No. 61/042,593 filed on Apr. 4, 2008, naming Martin Flueck as inventor and designated by attorney docket no. FLK-1001-PV. The instant patent application also claims the benefit of U.S. provisional patent application No. 61/075,239 designated by attorney docket no. FLK-1001-PV2. The foregoing provisional patent application No. 61/042,593 is entitled OVEREXPRESSION AND LOAD-INDUCIBLE ROLE OF FOCAL ADHESION SIGNALING FOR THE REGULATION OF MUSCLE REPAIR and the provisional patent application No. 61/075,239 is entitled THE REGULATION OF MUSCLE REPAIR. The entire content of each of the foregoing patent applications is incorporated herein by reference, including, without limitation, all text, tables and drawings.

### FIELD OF THE INVENTION

The invention pertains generally to mechanotransduction, and more specifically to focal adhesion signaling in muscles.

### BACKGROUND

Skeletal muscles naturally repair themselves efficiently after injury. However, with muscle damage from overuse during exercise, surgery or trauma or other muscle disorders such as Duchenne Muscular Dystrophy or Ehlers Danlos syndrome and other degenerative muscle diseases, normal repair functions can either be overwhelmed or cannot cope with disease progression to promote muscle repair. Physical therapy is sometimes prescribed to patients with the hope that exercising will aid not only in restoring muscle function but also in preventing further muscle damage or atrophy. Physical therapy helps in maintaining muscle strength and flexibility. Physical aids such as braces or wheelchairs also encourage mobility maintainance. It is possible to use a localized load-inducible role of focal adhesion signaling for the regulation of muscle repair. Biochemical signals originating at sites of focal adhesions between muscle fibers are functionally involved in the mechano-dependent governance of muscle gene expression.

### SUMMARY

Described herein are methods for promoting improvements of motor function by combining contraction-related and pharmacological interventions which stimulate focal adhesion signaling. Presented herein is a method for improving muscle function in a subject, by enhancing focal adhesion signaling and muscle fiber adhesion in a muscle of a subject and providing a load to the muscle whereby the focal adhesion signaling is enhanced and the load is provided each in an amount effective to improve the function of the muscle.

Focal adhesion signaling may be enhanced by any known method in the art. One example of enhancement is by administration of a signaling pathway agonist. Another example of enhancement is by administration of a signaling pathway member.

Enhancing focal adhesion signaling via an agonist can be performed by using a pharmacological drug. Examples of such drugs are bombesin, vasopressin, endothelin, vascular endothelial growth factor, angiotensin 2, activators of integrin signaling, activators of G-protein signaling, and reactive oxygen species.

Enhancing focal adhesion signaling via a signaling pathway member may be performed by delivering a nucleic acid that encodes the member. The nucleic acid encoding the member may be a vector, a plasmid, or a recombinant viral vector. Furthermore, the nucleic acid may be operably linked to a control element capable of directing in vivo transcription of the nucleic acid. The signaling pathway member may be administered by delivering a protein that encodes the member. The signaling pathway member may be selected from any molecule that plays a role in focal adhesion signaling. Examples of such molecules are focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, or eukaryotic translation initiation factor 4E binding protein I (eIF4E-BP1). The nucleic acid selected may be a focal adhesion kinase (FAK) sequence that is identical to or substantially identical to a fragment of an amino acid sequence encoded by SEQ ID No. 1.

The signaling pathway member may also comprise a detectable tag. The tag may be an epitope tag, a fluorescent tag, an affinity tag, a solubilization tag, or a chromatography tag.

The signaling pathway agonist or member may be administered to the subject by any known means in the art such as oral, rectal, transmucosal, transdermal, pulmonary, ophthalmic, intestinal, intramuscular, subcutaneous, intravenous, intramedullary, intrathecal, direct intraventricular, intraperitoneal, intranasal, or intraocular means.

The focal adhesion signaling may be enhanced after load is provided to the muscle. Or the load may be provided to the muscle after focal adhesion signaling is enhanced.

The muscle wherein focal adhesion signaling is enhanced may be skeletal, cardiac, smooth, slow oxidative fibers, fast oxidative fibers or fast glycolytic fibers.

Also provided herein is a method for determining whether a subject will respond to a treatment for improving muscle function, which includes measuring the activity of a focal adhesion signaling pathway member in a sample from a subject who has undergone or will undergo a treatment for muscle function that comprises (i) enhancing focal adhesion signaling in a muscle of a subject; and (ii) providing a load to the muscle; and determining whether the subject will respond to the treatment based on the measured activity. The focal adhesion signaling pathway member is selected from the group consisting of focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1). The S6 kinase level may measure the amount of myogenic effector proteins myo G and/or myo D, amount of S6 kinase RNA, the amount of S6 kinase protein, the degree of S6 kinase phosphorylation or the phosphotransfer activity of S6 kinase.

These and other embodiments are described hereafter in the Detailed Description and in the Claims.

### BRIEF DESCRIPTION OF THE DRAWINGS

FIG. 1 shows a sketch of how direct mechano-transduction and alterations in mechanical stress are mediated through focal adhesion kinase (FAK) towards enhanced protein translation.

FIG. 2 shows FAK staining of a positive fiber in a cross-section from TA muscle with pCMVFAK plasmid overexpression in a cage control animal.

FIG. 3 shows FAK overexpression in supernatant fractions of mouse TA muscle of hindlimb suspended and cage control mice.

FIG. 4 Effect of FAK overexpression on p70S6 Kinase protein phosphorylation on Ser411.

FIG. 5 Effect of FAK overexpression on eIF4E-BP1 phosphorylation on Thr37/46.

FIG. 6 shows the experimental design of the gene electrotransfer (A) and loading regimes (B).

FIG. 7 shows FAK overexpression in m. solei of cage controls.

FIG. 8 shows a gene ontology map.

FIG. 9 shows contractile consequences of FAK overexpression and functional overload.

FIG. 10 shows the effect of FAK overexpression in cage controls and during un- and reloading on protein expression of FAK, COX 1, COX4, MHC I and MHCII.

FIG. 11 shows FAK and MHC protein expression in single fibres.

FIG. 12 shows regulation of FAK.

FIG. 13 shows the plasmid pCMV-Myc used to clone chicken FAK.

FIG. 14A shows expression of TNC isoform in wildtype mice leg muscles and 14B shows TNC expression in lung, brain and skin.

FIG. 15 shows TNC-deficient mice with reduced mass of the pure fast muscle type tibialis anterior and extensor digitorum longus. FIG. 15B shows in TNC-deficient mice reduction of mean cross-sectional area for fast-type muscle fibers in extensor digitorum longus and soleus muscles.

FIG. 16A shows the deconditioned and reloaded soleus muscle of TNC-deficient mice and control mice. FIG. 16B shows transcripts which are significantly dependent on TNC-genotype of the one-day reloading response in one-year-old mice. FIG. 16C shows change in mRNA levels for TNC, cyclin A, myoG and myoD in control and TNC-deficient mice.

FIGS. 17A & B shows TNC overexpression in right muscles over 7 days. FIG. 17C shows levels of cyclin A, myoG and myoD.

FIG. 18 shows protein expression of TNC after electro transfer in TNC-deficient mice (A) and wild type (B), levels of expression (C) and staining with antibody (D).

FIG. 19 shows diagram for muscle repair.

FIG. 20 shows DNA-sequencing of the modified TNC gene.

FIG. 21 shows PCR-based genotyping for wild type and TNC-mice.

FIGS. 22A & B shows tenascin-W protein in soleus muscle of wildtype and TNC-deficient mice.

### DETAILED DESCRIPTION

The present invention relates to the mechanotransduction system within muscles, and to the use of the mechano-transducer focal adhesion kinase (FAK) in association with load-dependent stimulation of focal adhesion signaling for the repair/improvement of muscles. Activation of focal adhesion signaling within muscles may be achieved by gene transfer to increase the potential for focal adhesion signaling in myofibers via overexpression of focal adhesion components or by agonist activation of focal adhesion signalling or coupled pathways. Promotion of signalling within muscle cells may involve, but are not limited to, such proteins as integrin receptors (i.e RDG peptides), seven transmembrane receptors, tenascin-c, bombesin, or reactive oxygen species. The present invention may also serve as a preventative therapy for subjects diagnosed with a muscle related disease in order to slow progression of the disease. In addition the technology might be used in experimental animal models to quantify and optimize muscle repair via improved material properties of muscle related to lateral force transmission via reinforced muscle fibre adhesion. Furthermore, the present invention is especially important in special applications such as in sports medicine where specific muscle groups can be strengthened to decrease the impact of injuries or surgery when traditional exercise is not possible.

The term “subject” as used herein includes, but is not limited to, an organism or animal; a mammal, including, e.g., a human, non-human primate (e.g., monkey), mouse, pig, cow, goat, rabbit, rat, guinea pig, hamster, horse, sheep, or other non-human mammal; a non-mammal, including, e.g., a non-mammalian vertebrate, such as a bird (e.g., a chicken or duck) or a fish, and a non-mammalian invertebrate.

### Muscle Disease and Dystrophies

Muscle disease encompasses any of the diseases and disorders that affect the human muscle system. These diseases include myopathy, fibromyalgia, dermatomyositis, polymyositis, rhabdomyolysis and musuclar dystrophy. The muscular dystrophies (MD) are a group of more than 30 genetic diseases characterized by progressive weakness and degeneration of the skeletal muscles that control movement. Some forms of MD are seen in infancy or childhood, while others may not appear until middle age or later. The disorders differ in terms of the distribution and extent of muscle weakness (some forms of MD also affect cardiac muscle), age of onset, rate of progression, and pattern of inheritance.

Symptoms vary with the different types of muscular dystrophy. Some types, such as Duchenne muscular dystrophy, are deadly, while other types cause little disability and are associated with normal life span.

The muscles affected vary, but can be around the pelvis, shoulder, face or elsewhere. Muscular dystrophy can affect adults, but the more severe forms tend to occur in early childhood. Another type of MD is Ehlers-Danlos Syndrome (EDS) which weakens connective tissues such as collagen. Connective tissues are proteins that support skin, bones, blood vessels and other organs. Symptoms of EDS include loose joints, fragile, small blood vessels, abnormal scar formation and wound healing and soft, velvetly, stretchy skin that bruises easily.

Other symptoms of muscular dystrophies include muscle weakness that slowly gets worse, mental retardation (only present in some types of the condition), hypotonia, joint contractures (clubfoot, clawhand, or others), scoliosis (curved spine). And some types of muscular dystrophy involve the heart muscle, causing cardiomyopathy or arrhythmias.

There is no specific treatment to stop or reverse any form of MD. Treatment may include physical therapy, respiratory therapy, speech therapy, orthopedic appliances used for support, and corrective orthopedic surgery. Drug therapy includes corticosteroids to slow muscle degeneration, anticonvulsants to control seizures and some muscle activity, immunosuppressants to delay some damage to dying muscle cells, and antibiotics to fight respiratory infections. Some individuals may benefit from occupational therapy and assistive technology. Some patients may need assisted ventilation to treat respiratory muscle weakness and a pacemaker for cardiac abnormalities.

The prognosis for subjects with MD varies according to the type and progression of the disorder. Some cases may be mild and progress very slowly over a normal lifespan, while others produce severe muscle weakness, functional disability, and loss of the ability to walk. Some children with MD die in infancy while others live into adulthood with only moderate disability.

Focal Adhesion Signaling within Muscles

Mechanotransduction, or force-initiated signal transduction, is the process by which cells convert mechanical stimuli into a chemical response. Mechanical forces, through the initiation of signal transduction, play a critical role in cellular development (Grill et al. 2001 Nature 409:630 633), wound healing (Timmenga et al. 1991 Br. J. Plast. Surg. 44:514 519), cell growth (Damien et al. 2000 J. Bone Miner. Res. 15:2169 2177; Chen et al. 1997 Science 276:1425 1428), tissue remodeling (Grodzinsky et al. 2000 “Cartilage tissue remodeling in response to mechanical forces,” Annu. Rev. Biomed. Eng. 2:691 713), and sensory functions, such as touch and hearing. As such, understanding the mechanism of and identifying the key proteins in mechanotransduction may be useful for, inter alia, the treatment of wounds (e.g. treatment of burns, injuries and post-surgical lesions), the treatment of cancer through the control of cell growth, the healing of bone fractures, and the treatment of weakened muscle, which can be found in injury, aging, or muscle diseases such as muscular dystrophy and Ehlers Danlos syndrome. Examples of proteins that play a role in the mechanotraduction signalling pathway include focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

Focal adhesion/contacts are sites found on the plasma membrane where intracellular cytoskeletal elements come into contact with ECM proteins. Proteins localized to the focal adhesions/contacts, include focal adhesion kinase (FAK), paxillin, vinculin and integrins. Cells adhere tightly to the underlying substrate and the ECM proteins at focal adhesions. This adhesion is mediated by the integrin family of heterotrimeric cell surface receptors. In addition, actin filaments appear to be bundled by integrin receptors at the focal adhesions (as reviewed by Burridge et al. 1990, Cell Differ Dev December 2; 32(3):337 42). Thus, it has been hypothesized that focal adhesions may also serve at the site of mechanotransduction. Various studies show that mechanotransduction may occur at focal adhesions through the induction of changes to integrin-cytoskeletal bonds (Choquet et al. 1997 Cell 88:39 48) and cause redistribution of proteins to focal adhesions (Balaban et al. 2001 Nat. Cell Biol. 3:466 472).

Although it is not well understood in intact tissue, integrin-based focal adhesion complexes (FACs) are a major path for the integration of mechanical distortions into biochemical responses inside the cell (Davies et al., 1997; MacKenna et al., 2000). FACs are highly organized functional entities of cytoskeletal and signalling proteins of the plasma membrane which relay the extracellular matrix (ECM) to the cell interior (Burridge and Chrzanowska-Wodnicka, 1996; Ingber, 1997). In this context, activation of the integrin-associated focal adhesion kinase (FAK) via phosphorylation reflects the mechano-chemical coupling between mechanical stimulation of integrins and intracellular signalling (Davies et al., 1997; Giannone and Sheetz, 2006; Shyy and Chien, 1997). Post-translational modifications of tyrosine 397 in FAK's kinase domain augment the phosphotransfer activity of FAK towards associated cytoskeletal and signalling proteins (Guan, 1997; Hamadi et al., 2005; Pirone et al., 2006). This induces a cascade of reactions which regulate FAC turnover and may propagate inside the cell to activate downstream gene expression (for reviews see Guan, 1997; Parsons, 2003; Schlaepfer et al., 2004). In addition, there is evidence for an important control of FAK's function via its C-terminal focal adhesion targeting domain (Hildebrand et al., 1993; Ilic et al., 1995; Parsons, 2003; Richardson and Parsons, 1996). This is indicated by the altered cellular distribution of FAK, enhanced focal adhesion turnover and gene expression upon the exogenous overexpression of the FAK-related non-kinase (FRNK) C-terminal splice-form of FAK (Ilic et al., 1995; Mansour et al., 2004; Taylor et al., 2001; Yamada et al., 2005).

The physiological implication of focal-adhesion signalling in mechano-chemical transduction to downstream gene expression in vivo has not been addressed experimentally in a normally developed tissue (Hecker and Gladson, 2003). This is explained by lethal effects, developmental aberrations and compensatory processes resulting from the genetic ablation of focal adhesion components in the germline (Erickson, 1993; Ilic et al., 1995; Li et al., 1997). Since cultured cells do rarely show full differentiation of the adhesion-dependent link between the extracellular and cytoskeletal compartment conclusion on adhesion-dependent tissue control can not necessarily be extrapolated from isolated cell systems (Maniura-Weber et al., 2004; Sanes and Lawrence, 1983). Critical distinctions therefore apply for mechano-transduction via “chemical” focal adhesion signalling in cultures versus intact tissues.

### Tenascin-C (TNC)

Tenascin-C (TNC) is a hexameric extracellular matrix molecule which assembles from differently-spliced isoforms. TNC is expressed only in tissue undergoing active remodeling and in locations subject to high mechanical stress. TNC expression in musculoskeletal tissues is load-dependent and reversible. It has been suggested that micro-damage contributes to mechano-regulation of TNC expression. TNC exerts a strong anabolic and proliferative effect on interstitial and myogenic cells grown in culture. This biological activity is mediated by TNC's de-adhesive property which relieves cells from the growth inhibitory influence of substrate attachment. The transition to an intermediate adhesive state has been suggested to facilitate the expression of genes specific for tissue repair and adaptation. This view is supported by the de novo accumulation of TNC in muscle connective tissue after damaging muscle loading and the correlation of ectopic TNC protein with the expression of growth-related genes during muscle fiber regeneration. These observations suggest that TNC-mediated focal de-adhesion contributes to cell repair in mechano-sensitive tissues. The functional role of TNC in tissue morphogenesis remains unclear mainly because transgenic mice engineered for a TNC-deficiency show only subtle phenotypic defects. The reported pathologies in transgenic mouse lines include, amongst others, reduced neo-vascularization and cell migration in injured muscle tissue and mechanically-stressed corneal wounds. It is possible that the aberrations in TNC-deficient mice are masked to some extent by the permissive expression of an aberrant TNC variant. Possibly this ambiguity relates to the strategy employed to abolish production of the extracellular TNC protein via the disruption of the N-terminal signal sequence for protein export via the Golgi-apparatus. This genetic manipulation would leave intact downstream translation initiation sites for the production of shortened TNC variants. In cells residing in tissues that are normally exposed to mechanical stress, proteins can exit the cytoplasm by a diffusion-mediated route after plasma membrane disruption. The physiological implication of such a mechanism for TNC's role in tissue repair and the minor phenotype of transgenic mice with deficient TNC secretion are not understood.

TNC is associated with tissue remodeling and the present invention demonstrates that TNC deficient mice would demonstrate defects in the repair of damaged leg muscles which would be of functional significance since this tissue is subjected to frequent cycles of mechanical damage and regeneration.

TNC-deficient mice demonstrated a blunted expression of the large TNC isoform and selective atrophy of fast muscle fibers associated with a defective fast myogenic expression response to a damaging mechanical challenge. Transcript profiling mapped a set of de-adhesion, angiogenesis and wound healing regulators as TNC expression targets in striated muscle. Their expression correlated with the residual expression of a damage associated 200-kDa protein which resembled the small TNC isoform. Somatic Knock-in of TNC in fast muscle fibers confirmed the activation of a complex expression program of interstitial and slow myofiber repair by myofiber-derived TNC. Embodiments presented herein show for the first time that a TNC-orchestrated molecular pathway integrates muscle repair into the load-dependent control of the striated muscle phenotype.

The present invention presents a multilevel approach which combines the monitoring of damage related changes in muscles of both TNC-deficient and TNC Knock-in mice. The leg muscles are particularly suitable for this approach since they are amenable to physiological modulation of their mechanical activity and because they are accessible to somatic transgenesis. The pathways underlying TNC action were identified by monitoring aberrant transcript expression of muscle-relevant gene ontologies in anti-gravitational soleus muscle of TNC-deficient mice in response to the damaging mechanical stimulus of reloading after deconditioning bearing in mind the possible expression of an aberrant TNC variant. The control of selected TNC dependent gene products was verified ad hoc with muscle fiber-targeted somatic Knock-in experiments.

### Exercise and Muscle Strength

It is fairly well understood that general muscle strength, condition, and tone has a significant impact on overall health which is often undermined by inadequate exercise. The regularity with which a muscle is used, as well as the duration and intensity of its activity, affects the properties of the muscle. If the neurons to a skeletal muscle are severed or otherwise destroyed, the denervated muscle fibers will become progressively smaller in diameter, and the amount of contractile proteins they contain will decrease. A muscle can also atrophy with its nerve supply intact if the muscle is not used for a long period of time, as when a broken arm or leg is immobilized in a cast (Fluck and Hoppeler, 2003).

In contrast to the decrease in muscle mass that results from a lack of neural stimulation, increased amounts of contractile activity or exercise, can produce an increase in the size (hypertrophy) of muscle fibers as well as changes in their chemical composition. Since the number of fibers in a muscle remains essentially constant throughout adult life, the changes in muscle size with atrophy and hypertrophy do not result from changes in the number of muscle fibers but in the metabolic capacity and size of each fiber.

The force exerted on an object by a contracting muscle is known as muscle tension, and the force exerted on the muscle by the weight of an object is the load. Muscle tension and load are opposing forces. Whether or not force generation leads to fiber shortening depends on the relative magnitudes of the tension and the load. In order for muscle fibers to shorten and therefore move a load, muscle tension must become and remain slightly greater than the opposing load.

Exercise that is of relatively low intensity but of long duration (aerobic exercise) such as long-distance running and swimming, produces increases in the number of mitochondria in the fast and slow oxidative fibers, which are recruited in this type of activity. In addition, there is an increase in the number of capillaries around these fibers. All these changes lead to an increase in the capacity for endurance activity with a minimum of fatigue. Endurance exercises produce changes not only in the skeletal muscles but also in the respiratory and circulatory systems, changes that improve the delivery of oxygen and fuel molecules to the muscle.

In contrast, short-duration, high-intensity exercise (strength training) such as weight lifting, affects primarily the fast-glycolytic fibers, which are briefly recruited during strong contractions. These fibers undergo an increase in fiber diameter resulting from the increased synthesis of actin and myosin filaments, which form more myofibrils. In addition, the glycolytic activity is increased because of the increased synthesis of glycolytic enzymes. The result of such high-intensity exercise is an increase in the strength of the muscle.

Exercise produces little change in the types of myosin formed by the fibers and thus little change in the proportions of fast and slow fibers in a muscle. As described above, however, exercise does change the rates at which metabolic enzymes are synthesized, leading to changes in the proportion of oxidative and glycolytic fibers within a muscle.

The signals responsible for all these changes in muscle with different types of activity are unknown. They are related to the frequency and intensity of the contractile activity in the muscle fibers and thus to the pattern of action potentials produced in the muscle over an extended period of time. Similar adaptive changes in cell size, number, or capacity for functional activity are seen in many other organs in the body in response to increased demands on the tissue.

The changes in muscle occur slowly over a period of weeks in response to repeated periods of exercise. If exercise is stopped, the changes in the muscle that occurred as a result of the exercise will slowly revert to their state before exercise.

In most accounts, cultural belief in the necessity of physical fitness programs for the general population is a relatively new concept in history. The concept of conducting a regular physical fitness program emerged in the first half of the 20th Century and popularity of such programs has steadily progressed. The need for regular physical fitness may be a reflection of the fundamental shift from agrarian to industrial and urban societies over the previous 100 years. As the need to hunt, cultivate, and gather food has been reduced within a culture, the amount of physical activity associated with these activities has correspondingly decreased.

The decrease in overall physical activity has led to the development of numerous public health concerns. Currently, obesity and musculoskeletal disorders are two of the most pressing health problems in the United States. Lack of or reduced physical activity may be a significant factor in both of these conditions as well as many other health related conditions.

There are a variety of ways in which muscles may be damaged. One of ordinary skill in the art can identify the various types of muscles that can be damaged as well as the various ways damage that can be done to a muscle. Examples of different muscle types include, but are not limited to, skeletal, cardiac, smooth, slow oxidative fibers, fast oxidative fibers and fast glycolytic fibers. Examples of different ways damage is done to a muscle include, but are not limited to, crushing, tearing, excess straining, laceration, internal duress, and inflammation.

### B. Enhancing Focal Adhesion Signaling

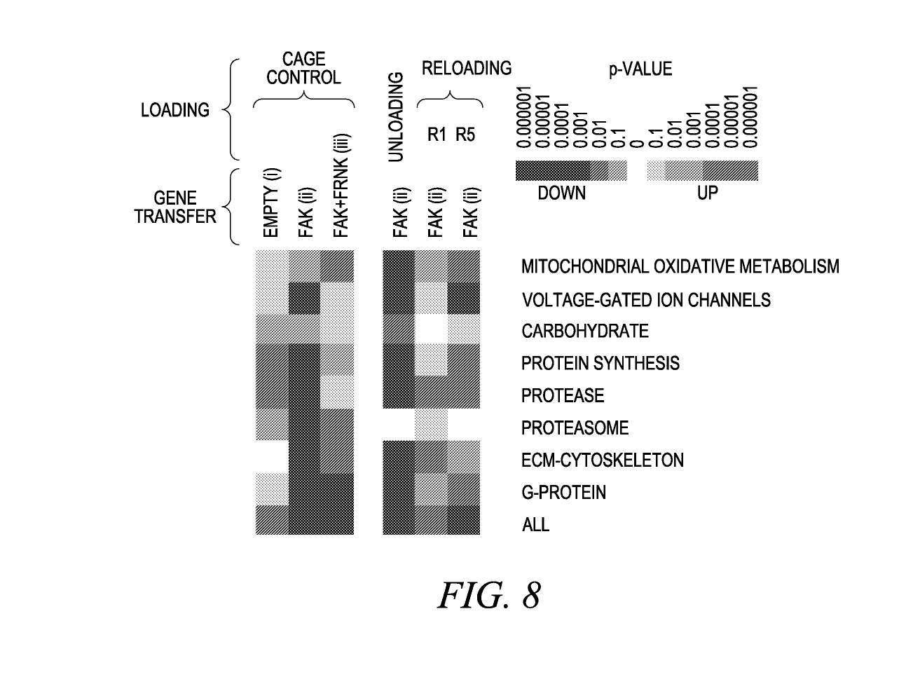

Repair mechanisms importantly contribute to muscle plasticity by promoting the replacement of damaged muscle structures and growth. The relevant molecular mechanisms underlying this control in a physiological context are not well defined. Consequently, current methodologies are not efficient to promote viable tissue growth/repair because the synergistic growth signals of the broad-specific physiological stimuli of muscle plasticity are not targeted. These routes of myocellular signalling have been selected through evolution to provide the powerful stimulation of the biological processes which interplay to condition muscle traits. Such knowledge is the key to the development of effective therapeutic interventions to promote repair and growth of muscle. Striated muscle offers as a well characterized model for the investigation of the mechanodependent growth and differentiation control in a fully-differentiated tissue. Although any muscle systems may be used that one of ordinary skill in the art can identify examples of which include but are not limited to skeletal, cardiac, smooth, slow oxidative fibers, fast oxidative fibers and fast glycolytic fibers which may provide the research environment into mechanotransduction signalling. Both, alterations in muscle loading and recruitment induce pronounced adjustments of the contractile and metabolic makeup of muscle tissue (Fluck and Hoppeler, 2003; Goldspink, 1999; Kjaer, 2004). In this respect, prolonged reductions in weight-bearing (unloading) by hindlimb-suspension bring about metabolic and contractile deconditioning of anti-gravitational soleus muscle by wasting (atrophy) and slow-to-fast transformation which can be reversed by reloading (reviewed by Fluck et al., 2005). Similarly, growth of muscle fibers (hypertrophy) can be induced experimentally within a week by chronic functional overload after tenotomy or ablation of agonist muscles (Gordon et al., 2001). Previous transcript profiling studies invoked the existence of a load-induced master regulatory pathway in the expression control of gene ontologies underlying metabolic differentiation of soleus muscle (Fluck et al., 2005). This was indicated by global expressional regulation of transcripts involved in protein turnover, oxidative metabolism and muscle excitation during the reestablishment of the soleus muscle phenotype with reloading after a period of unloading (for example, see FIG. 8). Thus muscle plasticity models studies offers well-defined endpoints for mechanistic investigations on the regulation of mechano-dependent myocellular remodelling. The recent advent of gene electrotransfer, also offers to overcome critical limitations in the study of signalling in striated muscle (Durieux et al., 2004; Durieux et al., 2002). This mode of somatic transgenesis allows interfering with biological processes in an unperturbed animal via targeted overexpression of exogenous factors in muscle fibres (Klossner). Importantly, gene electrotransfer allows control over technical limitations such as associated fibre damage via the inclusion of tissue-internal, and intra-animal controls (Gronevik et al., 2005; Rizzuto et al., 1999; Tupling et al., 2002).

Comparative studies comply with an involvement of the regulated activation of sarcolemmal FAK in load-dependent muscle plasticity. This is indicated by i) FAK's localization to the major sites of lateral mechano-sensation in contractile cells, i.e. sarcolemmal focal adhesions (costameres) (Bloch and Gonzalez-Serratos, 2003; Ervasti, 2003; Quach and Rando, 2006; Samarel, 2005), ii) the reciprocal control of tyrosine phosphorylation, kinase activity and expression of FAK protein in vertebrate muscle by functional overload and unloading (Fluck et al., 1999; Gordon et al., 2001), and iii) the enhanced localization of FAK to the sarcolemma in compliance with the frequency of fibre recruitment (Fluck et al., 2002). This suggests that FAK could be the mechano-dependent switch for the metabolic and contractile differentiation of frequently recruited fibres with a slow-oxidative phenotype and elevated protein turnover (reviewed in Fluck et al., 2002; Habets et al., 1999). This notion is supported by the demonstrated activation of cell differentiation and hypertrophy pathways by FAK in cardiac and skeletal muscle cultures (Clemente et al., 2005; Fonseca et al., 2005; Kovacic Milivojevic et al., 2001; Nadruz et al., 2005; Pham et al., 2000; Quach and Rando, 2006; Sastry et al., 1999). Results in culture also hint that relocalization of FAK to sarcolemmal locations (costameres) underlies mechano-transduction in contractile cells (Fonseca et al., 2005). The extent to which the sarcoplasmic and sarcolemmal FAK pools in contractile cells (Fonseca et al., 2005) intervene in the mechano-dependent specialization of metabolic and contractile muscle features in vivo is not understood.

The present invention is based in part on the observation that myocellular FAK constitutes a load-dependent governor of the expression program underpinning the phenotypic differentiation of frequently recruited muscle fibres. To demonstrate this, FAK's role in mechano-transduction is shown via interference with load modulated muscle signalling by constitutive, somatic overexpression of the chicken FAK homologue (SEQ ID NO. 1) and competition with its autonomous inhibitor, FRNK, in anti-gravitational muscle. FAK regulates transcript levels of gene ontologies underlying differentiation of slow, fatigue-resistant fibres in rat soleus muscle such as slowed excitation-contraction, elevated oxidative metabolism and the cytoskeleton (Bozyczko et al., 1989), as well as elevated protein turnover (Habets et al., 1999) has been demonstrated. Overexpression and electrotransfer-associated bias was controlled via a design allowing paired comparison to empty- and non-transfected muscles and muscle fibres. FAK-regulated pathways were identified from perturbed muscle transcript expression in conjunction to morphological and contractile adjustments of muscle fibres and changes in FAK's phosphorylation and localization in the transgenic muscle. Mechano-dependent soleus muscle plasticity was induced in the hindlimb unloading-reloading model with the time course of the adaptive response (Fluck et al., 2005; Gordon et al., 2001). Physiological testing was completed with the tenotomy model to produce larger increases in muscle loading and reduce inferences from the silencing of the constitutive promoter activity of the expression plasmid with the long durations of unloading prior to the mechanical stimulus (Brooks et al., 2004). Due to the selective targeting of exogenous protein expression in a proportion of fibres, the muscular changes induced by FAK-overexpression would be small but could be exposed with this quantitative approach.

Overexpression of a molecule involved in focal adhesion signaling to enhance muscle repair may be performed in a variety of ways. One of ordinary skill in the art would recognize the various methods. The overexpression of a molecule may be done in a variety of forms which include but are not limited to a nucleic acid, protein, drug, or component thereof. Routes of activating these molecules may also be performed in a variety of ways which include but are not limited to, administering to a subject a phosphorylated FAK protein, an unphosphorylated FAK protein or a molecule that aids in phosphorylation. The sections below will further go into detail two optional methods of overexpressing molecules within the focal adhesion signaling pathway.

### Nucleic Acid Constructs

FAK Molecules The term “FAK molecule” refers to a molecule, such as a protein, polypeptide, nucleic acid or expression vector, for example, comprising a native FAK sequence, or fragment thereof or substantially identical variant of the foregoing. A FAK molecule sometimes is capable of enhancing antigen presenting cell longevity and immunogenicity when expressed in an antigen presenting cell in such a manner that it is membrane-targeted, and constitutively active in certain embodiments. A FAK molecule also may include other portions in addition to the FAK sequence, and in such embodiments, the FAK sequence in the FAK molecule sometimes is referred to herein as an “FAK portion” or “FAK region.” Additional sequences that may be included optionally in an FAK molecule are described herein, such as a membrane association sequence and/or a multimerization sequence, for example.

A FAK sequence may be a native FAK sequence, a fragment of an FAK sequence or a substantially identical variant of the foregoing. A FAK sequence sometimes is mammalian (e.g., mouse or human), or a fragment or variant sequence thereof. Examples of native polynucleotide sequences that encode FAK polypeptides include, but are not limited to, SEQ ID NO: 1 (chicken FAK) and FAK homologs from other species and including FAK oncogenic viral sequences.

As noted above, FAK sequences include FAK fragment sequences. A FAK fragment sequence may lack one or more nucleotides, amino acids or regions, the latter of which may be a functional region or domain. A FAK fragment sequence can include one or more functional regions, and may lack one or more functional regions compared to a native FAK sequence. Where a FAK fragment sequence includes one or more functional regions, the region may be flanked on each side by a native amino acid sequence from a native FAK sequence. In certain embodiments, a FAK amino acid fragment sequence is 10 or more, 15 or more, 20 or more, 25 or more, 50 or more, 100 or more, 200 or more, 300 or more, 400 or more or 450 or more amino acids from a native FAK protein. A FAK molecule sometimes includes an FAK protein kinase catalytic domain, and therefore sometimes is capable of catalyzing Tyr protein phosphorylation. A FAK fragment can exclude a PH domain or includes a modified PH domain. A modified PH domain may be truncated or mutated, generated by using standard mutagenesis, insertions, deletions, or substitutions, and the modified form may or may not be functional.

FAK sequences include homologs, alternative transcripts, alleles, functionally equivalent fragments, variants, and analogs of native FAK sequences (e.g., nucleotide sequences described herein). The term “substantially identical variant” as used herein refers to a nucleotide or amino acid sequence sharing sequence identity to a nucleotide sequence or amino acid sequence of FAK or another molecule described herein (e.g., membrane association region). Included are nucleotide sequences or amino acid sequences 55% or more, 60% or more, 65% or more, 70% or more, 75% or more, 80% or more, 85% or more, 90% or more, 95% or more (each sometimes within a 1%, 2%, 3% or 4% variability) identical to a nucleotide sequence or encoded amino acid sequence described herein, or has one to ten nucleotide or amino acid substitutions. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, sometimes 60% or more, and more sometimes 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences. Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters sometimes used is a Blossum 62 scoring matrix with a gap open penalty of 12, a gap extend penalty of 4, and a frameshift gap penalty of 5.

Another manner for determining whether two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Sometimes, stringent hybridization conditions are hybridization in 6× sodium chloride/sodium citrate (SSC) at about 45° C., followed by one or more washes in 0.2×SSC, 0.1% SDS at 65° C. Other times, stringency conditions are 0.5M sodium phosphate, 7% SDS at 65° C., followed by one or more washes at 0.2×SSC, 1% SDS at 65° C.

An example of a substantially identical nucleotide sequence to a base nucleotide sequence described herein is one that has a different nucleotide sequence but still encodes the same amino acid sequence encoded by the base nucleotide sequence. Another example is a nucleotide sequence that encodes a protein having an amino acid sequence 70% or more identical to, sometimes 75% or more, 80% or more, or 85% or more identical to, and sometimes 90% to 99% identical to an amino acid sequence encoded by the base nucleotide sequence.

Nucleotide sequences and encoded amino acid sequences described herein can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences described herein. BLAST polypeptide searches can be performed with the XBLAST program, score=50, wordlength=3 to obtain amino acid sequences homologous to those encoded by nucleotide sequences described herein. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http World Wide Web address ncbi.nlm.nih.gov). Thus, a protein having a substantially identical amino acid sequence to (i) an amino acid sequence described herein or (ii) an amino acid sequence encoded by a nucleotide sequence described herein, identified by a query sequence search can be considered a substantially identical sequence.

Substantially identical nucleotide sequences may include altered codons for enhancing expression of an amino acid sequence in a particular expression system. One or more codons may be altered, and sometimes 10% or more or 20% or more of the codons are altered for optimized expression in an expression system that may include bacteria (e.g., E. coli.), yeast (e.g., S. cervesiae), human (e.g., 293 cells or antigen presenting cells), insect, or rodent (e.g., hamster) cells (e.g., antigen presenting cells).

A FAK protein, polypeptide or fragment variant can include one or more amino acid substitutions, deletions or insertions. Any amino acid may be substituted by a conservative or non-conservative substitution. For example, phosphorylatable amino acids (e.g., serine, threonine or tyrosine) in a FAK protein or fragment may be modified (e.g., deleted or substituted with an amino acid that cannot be phosphorylated).

A FAK protein, polypeptide or fragment variant may contain one or more unnatural amino acids. Unnatural amino acids include but are not limited to D-isomer amino acids, ornithine, diaminobutyric acid, norleucine, pyrylalanine, thienylalanine, naphthylalanine and phenylglycine, alpha and alpha-disubstituted amino acids, N-alkyl amino acids, lactic acid, halide derivatives of natural amino acids such as trifluorotyrosine, p-Cl-phenylalanine, p-Br-phenylalanine, p-I-phenylalanine, L-allyl-glycine, beta-alanine, L-alpha-amino butyric acid, L-gamma-amino butyric acid, L-alpha-amino isobutyric acid, L-epsilon-amino caproic acid, 7-amino heptanoic acid, L-methionine sulfone, L-norleucine, L-norvaline, p-nitro-L-phenylalanine, L-hydroxyproline, L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid, L-Phe (4-benzyl), 2,4-diaminobutyric acid, 4-aminobutyric acid (gamma-Abu), 2-amino butyric acid (alpha-Abu), 6-amino hexanoic acid (epsilon-Ahx), 2-amino isobutyric acid (Aib), 3-amino propionic acid, ornithine, norleucine, norvaline, hydroxyproline, sarcosine, citrulline, homocitrulline, cysteic acid, t-butylglycine, t-butylalanine, an amino acid derivatized with a heavy atom or heavy isotope (e.g., Au, deuterium, 15N; useful for synthesizing protein applicable to X-ray crystallographic structural analysis or nuclear magnetic resonance analysis), phenylglycine, cyclohexylalanine, fluoroamino acids, designer amino acids such as beta-methyl amino acids, Ca-methyl amino acids, Na-methyl amino acids, naphthyl alanine, and the like.

Cells are transfected or transformed with a nucleic acid having a FAK polynucleotide homologue sequence that encodes a molecule described herein. The nucleic acid bearing such a nucleotide sequence can be transferred into the muscle in a variety of manners, as described hereafter (e.g., delivery of a naked nucleic acid or encapsulation of the nucleic acid in a liposome or virus). Based on nucleotide sequences within the nucleic acid, a target nucleotide sequence encoding a FAK molecule and/or other target molecules may be stably integrated into the genomic DNA of the antigen presenting cell, in a random or non-random manner (e.g., knock-in), or may be transiently deposited to the antigen presenting cell.

Nucleic acids containing a FAK nucleotide sequence sometimes are referred to herein as “nucleic acid compositions.” A nucleic acid composition can be from any source or composition, such as DNA, cDNA, RNA or mRNA, for example, and can be in any suitable form (e.g., linear, circular, supercoiled, single-stranded, double-stranded, and the like). A nucleic acid composition sometimes is a plasmid, phage, autonomously replicating sequence (ARS), centromere, artificial chromosome or other nucleic acid able to replicate or be replicated in vitro or in a host cell (e.g., dendritic cell). Such nucleic acid compositions are selected for their ability to guide production of the desired protein or nucleic acid molecule. When desired, the nucleic acid composition can be altered as known in the art such that codons encode for a different amino acid than is normal, including unconventional or unnatural amino acids (including detectably labeled amino acids).

A nucleic acid composition can comprise certain elements sometimes selected according to the intended use of the nucleic acid. Any of the following elements can be included in or excluded from a nucleic acid composition. A nucleic acid composition, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and a selection element. A nucleic acid composition is provided with one or more of such elements and other elements may be inserted into the nucleic acid before the template is contacted with a transcription and/or translation system. In some embodiments, a provided nucleic acid composition comprises a promoter, 5′UTR, optional 3′UTR and insertion element(s) by which a target nucleotide sequence is inserted (i.e., cloned) into the template. In certain embodiments, a provided nucleic acid composition comprises a promoter, insertion element(s) and optional 3′UTR, and a 5′ UTR/target nucleotide sequence is inserted with an optional 3′UTR. The elements can be arranged in any order suitable for transcription and/or translation, and in some embodiments a nucleic acid composition comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

A promoter element typically is required for DNA synthesis and/or RNA synthesis. A promoter sometimes interacts with a RNA polymerase to generate message RNA suitable for translation of a protein, polypeptide or peptide. Promoter sequences are readily accessed and obtained by the artisan, and are readily adapted to nucleic acid compositions described herein. The particular promoter employed to control the expression of a polynucleotide sequence of interest is not believed to be important, so long as it is capable of directing the expression of the polynucleotide in the targeted cell. Thus, where a human cell is targeted, it is preferable to position the polynucleotide sequence-coding region adjacent to and under the control of a promoter that is capable of being expressed in a human cell. Generally speaking, such a promoter might include either a human or viral promoter. Examples of promoters include human cytomegalovirus (CMV) immediate early gene promoter, SV40 early promoter, Rous sarcoma virus long terminal repeat, beta.-actin, elongation factor 1-alpha (EF-1.alpha.), rat insulin promoter and glyceraldehyde-3-phosphate dehydrogenase. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

In some circumstances, it is desirable to regulate expression of a transgene in an immunotherapy vector. For example, different viral promoters with varying strengths of activity are utilized depending on the level of expression desired. In mammalian cells, the CMV immediate early promoter can be used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV can be used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus.

Tissue specific promoters sometimes are used to effect transcription in specific tissues or cells so as to reduce potential toxicity or undesirable effects to non-targeted tissues. For example, promoters such as the alpha myosin heavy chain (αMHC) promoter, directing expression to cardiac myocytes.

In certain indications, it is desirable to activate transcription at specific times after administration of the vector. Promoters that are hormone or cytokine regulatable can be utilized. Cytokine and inflammatory protein responsive promoters that can be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antityrpsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and gluccocorticoid inducible), Stromelysin (inducible by phorbol ester, interleukin-1 and EGF), alpha-2 macroglobulin and alpha-1 antichymotrypsin. CID promoters also can be utilized (Ho et al., 1996; Rivera et al., 1996). Full citations of certain documents referenced herein are in U.S. 20030144204, published Jul. 31, 2003.

Other inducible promoters are known and can be utilized. An ecdysone system (Invitrogen, Carlsbad, Calif.) is one such system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-responsive promoter, which drives expression of the gene of interest is on another plasmid. Engineering of this type of system into the gene transfer vector of interest would therefore be useful. Cotransfection of plasmids containing the gene of interest and the receptor monomers in the producer cell line would then allow for the production of the gene transfer vector without expression of a potentially toxic transgene. At the appropriate time, expression of the transgene could be activated with ecdysone or muristeron A. Another inducible system is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Offm system would be preferable so that the producer cells could be grown in the presence of tetracycline or doxycycline and prevent expression of a potentially toxic transgene, but when the vector is introduced to the patient, the gene expression would be induced constitutively.

It is envisioned that any of the above promoters alone or in combination with another can be useful according to the present invention depending on the action desired. In addition, this list of promoters should not be construed to be exhaustive or limiting, those of skill in the art will know of other promoters that are used in conjunction with the promoters and methods disclosed herein.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the transcription and/or translation system being utilized. A 5′ UTR sometimes comprises one or more of the following elements known to the artisan: enhancer sequence (e.g., Eukaryotic Promoter Data Base EPDB), translational enhancer sequence, transcription initiation site, transcription factor binding site, translation regulation site, translation initiation site, translation factor binding site, ribosome binding site, replicon, enhancer element, internal ribosome entry site (IRES), and silencer element.

A 5′UTR in the nucleic acid composition can comprise a translational enhancer nucleotide sequence. A translational enhancer nucleotide sequence sometimes is located between the promoter and the target nucleotide sequence in a nucleic acid composition. A translational enhancer sequence sometimes binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews 0004.1-0001.10 (2002); Gallie, Nucleic Acids Research 30: 3401-3411 (2002); Shaloiko et al., http address www.interscience.wiley.com, DOI: 10.1002/bit.20267; and Gallie et al., Nucleic Acids Research 15: 3257-3273 (1987)). A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid composition as a translational enhancer sequence (e.g., devoid of guanosine nucleotides and includes a 25 nucleotide long poly (CAA) central region). In some embodiments, a translational enhancer sequence comprises one or more ARC-1 or ARC-1 like sequence, such as one of the following nucleotide sequences GCCGGCGGAG, CUCAUAAGGU, GACUUUGAUU, CGGAACCCAA, AUACUCCCCC and CCUUGCGACC, or a substantially identical sequence thereof. In certain embodiments, a translational enhancer sequence comprises an IRES sequence, such as one or more of EMBL nucleotide sequences J04513, X87949, M95825, M12783, AF025841, AF013263, AF006822, M17169, M13440, M22427, D14838 and M17446, or a substantially identical nucleotide sequence thereof. An IRES sequence may be a type I IRES (e.g., from enterovirus (e.g., poliovirus), rhinovirus (e.g., human rhinovirus)), a type II IRES (e.g., from cardiovirus (e.g., encephalomyocraditis virus), aphthovirus (e.g., foot-and-mouth disease virus)), a type III IRES (e.g., from Hepatitis A virus) or other picornavirus sequence (e.g., Paulos et al. supra, and Jackson et al., RNA 1: 985-1000 (1995)).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the transcription and/or translation system being utilized. A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR can include a polyadenosine tail, and sometimes may not. If a polyadenosine tail is present, one or more adenosine moieties may be added or deleted from the native length (e.g., about 5, about 10, about 15, about 20, about 25, about 30, about 35, about 40, about 45 or about 50 adenosine moieties may be added or subtracted).

The term a “target nucleotide sequence” as used herein encodes a nucleic acid, peptide, polypeptide or protein of interest, and may be a ribonucleotide sequence or a deoxyribonucleotide sequence. A FAK nucleotide sequence (e.g., a sequence encoding a FAK homologue sequence) may be incorporated into a nucleic acid composition as a target nucleotide sequence. The term “nucleic acid” as used herein is generic to polydeoxyribonucleotides (containing 2′-deoxy-D-ribose or modified forms thereof), to polyribonucleotides (containing D-ribose or modified forms thereof), and to any other type of polynucleotide which is an N-glycoside of a purine or pyrimidine bases, or modified purine or pyrimidine bases. A target nucleic acid can include an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide or protein, which are sometimes referred to herein as “target peptides,” “target polypeptides” or “target proteins.” The term “protein” as used herein refers to a molecule having a sequence of amino acids linked by peptide bonds. This term includes fusion proteins, oligopeptides, polypeptides, cyclic peptides, polypeptides and polypeptide derivatives. A protein or polypeptide sometimes is of intracellular origin (e.g., located in the nucleus, cytosol, or interstitial space of host cells in vivo) and sometimes is a cell membrane protein in vivo.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A nucleic acid composition sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species, such as human, insect, nematode, bovine, equine, canine, feline, rat or mouse, for example. A FAK nucleotide sequence sometimes is utilized as an ORF herein, and sometimes a membrane association region-encoding nucleotide sequence is utilized as an ORF.

A nucleic acid composition sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid composition, thereby encoding a tag at the C-terminus or N-terminus of the protein or peptide encoded by the ORF. Any tag that does not abrogate or substantially reduce transcription and/or translation may be utilized and may be appropriately selected by the artisan. A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), AU1 (e.g., DTYRYI), V5 (e.g., GKPIPNPLLGLDST), c-MYC (e.g., EQKLISEEDL), HSV (e.g., QPELAPEDPED), influenza hemaglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His₆) or other sequence that chelates a metal (e.g., cobalt, zinc, copper) and/or a cysteine-rich sequence that binds to an arsenic-containing molecule. In certain embodiments, a cysteine-rich tag comprises the amino acid sequence CC-Xₙ-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His₆).

A tag sometimes conveniently binds to a binding partner. For example, some tags bind to an antibody (e.g., FLAG) and sometimes specifically bind to a small molecule. For example, a polyhistidine tag specifically chelates a bivalent metal, such as copper, zinc and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ (EDT_{2[}4′,5′-bis(1,3,2-dithioarsolan-2-yl)fluorescein-(1,2-ethanedithiol)₂]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”). Such antibodies and small molecules sometimes are linked to a solid phase for convenient isolation of the target protein or target peptide, as described in greater detail hereafter.

A tag sometimes comprises a sequence that localizes a translated protein or peptide to a component in a system, which is referred to as a “signal sequence” or “localization signal sequence” herein. A signal sequence sometimes is incorporated at the N-terminus of a target protein or target peptide, and sometimes is incorporated at the C-terminus. Examples of signal sequences are known to the artisan, are readily incorporated into a nucleic acid composition, and sometimes are selected according to the cells from which a cell-free extract is prepared. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondria targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A tag sometimes is directly adjacent to the amino acid sequence encoded by an ORF (i.e., there is no intervening sequence) and sometimes a tag is substantially adjacent to the ORF encoded amino acid sequence (e.g., an intervening sequence is present). An intervening sequence sometimes includes a recognition site for a protease, which is useful for cleaving a tag from a target protein or peptide. In some embodiments, the intervening sequence is cleaved by Factor Xa (e.g., recognition site I(E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and sometimes comprises a higher proportion of amino acids having relatively short side chains (e.g., glycine, alanine, serine and threonine).

A nucleic acid composition sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including but not limited to, supE, supP, supD, supF and supZ suppressors, which suppress the termination of translation of the amber stop codon; supB, gIT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a nucleic acid composition comprising a stop codon located between an ORF and a tag can yield a translated ORF alone when no suppressor tRNA is present in the translation system, and can yield a translated ORF-tag fusion when a suppressor tRNA is present in the system. In some embodiments, the stop codon is located 3′ of an insertion element or ORF and 5′ of a tag, and the stop codon sometimes is an amber codon. Suppressor tRNA sometimes are within a cell-free extract (e.g., the cell-free extract is prepared from cells that produce the suppressor tRNA), sometimes are added to the cell-free extract as isolated molecules, and sometimes are added to a cell-free extract as part of another extract. A provided suppressor tRNA sometimes is loaded with one of the twenty naturally occurring amino acids or an unnatural amino acid (described herein). Suppressor tRNA can be generated in cells transfected with a nucleic acid encoding the tRNA (e.g., a replication incompetent adenovirus containing the human tRNA-Ser suppressor gene can be transfected into cells). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address www.invitrogen.com/content/sfs/manuals/tagondemand_supernatant_man.pdf; Tag-On-Demand™ Gateway® Vector Instruction Manual, Version B, 20 Jun. 2003 at http address www.invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

Any convenient cloning strategy known to the artisan may be utilized to incorporate an element, such as an ORF, into a nucleic acid composition. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid composition, such as an oligonucleotide primer hybridization site for PCR, for example, and others described hereafter.

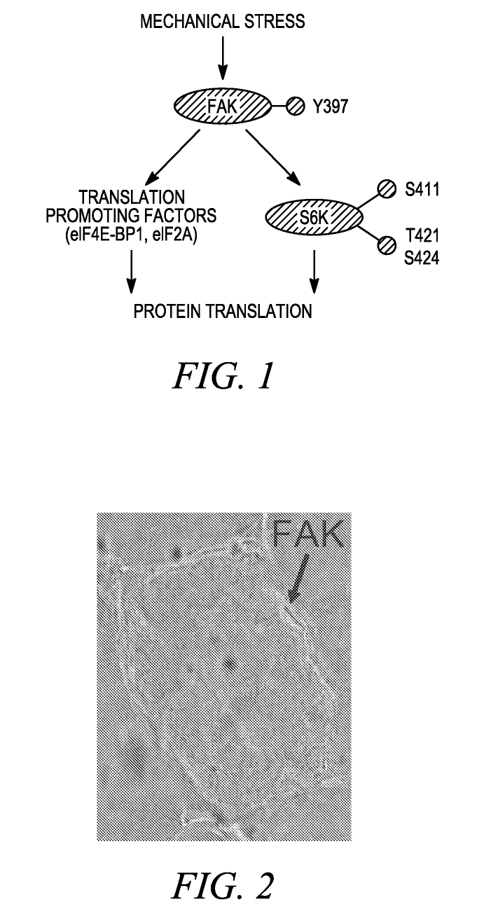

In some embodiments, the nucleic acid composition includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. No. 09/517,466, filed Mar. 2, 2000, and 09/732,914, filed Aug. 14, 2003, and in U.S. patent publication no. 2002-0007051-A1; Landy, Curr. Opin. Biotech. 3:699-707 (1993)). Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, sometimes based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

In certain embodiments, the nucleic acid composition includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO₄-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted sometimes is combined with topoisomerase-reacted template and thereby incorporated into the nucleic acid composition (e.g., http address www.invitrogen.com/downloads/F-13512_Topo_Flyer.pdf; http address at www.invitrogen.com/content/sfs/brochures/710_{—}021849%20_B_TOPOCloning_bro.pdf; TOPO TA Cloning® Kit and Zero Blunt® TOPO® Cloning Kit product information).

A nucleic acid composition sometimes contains one or more origin of replication (ORI) elements. In some embodiments, a template comprises two or more ORIs, where one functions efficiently in one organism (e.g., a bacterium) and another functions efficiently in another organism (e.g., a eukaryote). In some embodiments, an ORI may function efficiently in insect cells and another ORI may function efficiently in mammalian cells. A nucleic acid composition also sometimes includes one or more transcription regulation sites.

A nucleic acid composition sometimes includes one or more selection elements. Selection elements sometimes are utilized using known processes to determine whether a nucleic acid composition is included in a cell. In some embodiments, a nucleic acid composition includes two or more selection elements, where one functions efficiently in one organisms and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; nucleic acid segments that inhibit replication, partition or heritability of nucleic acid molecules that contain them; and/or nucleic acid segments that encode conditional replication functions, e.g., replication in certain hosts or host cell strains or under certain environmental conditions (e.g., temperature, nutritional conditions, and the like).

Certain nucleotide sequences sometimes are added to, modified or removed from one or more of the nucleic acid composition elements, such as the promoter, 5′UTR, target sequence, or 3′UTR elements, to enhance or potentially enhance transcription and/or translation before or after such elements are incorporated in a nucleic acid composition. In some embodiments, one or more of the following sequences may be modified or removed if they are present in a 5′UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures)); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′UTR (e.g., EMBL nucleotide sequences J04513, X87949, M95825, M12783, AF025841, AF013263, AF006822, M17169, M13440, M22427, D14838 and M17446 and substantially identical nucleotide sequences). An AU-rich element (ARE, e.g., AUUUA repeats) and/or splicing junction that follows a non-sense codon sometimes is removed from or modified in a 3′UTR. A polyadenosine tail sometimes is inserted into a 3′UTR if none is present, sometimes is removed if it is present, and adenosine moieties sometimes are added to or removed from a polyadenosine tail present in a 3′UTR. Thus, some embodiments are directed to a process comprising: determining whether any nucleotide sequences that reduce or potentially reduce translation efficiency are present in the elements, and removing or modifying one or more of such sequences if they are identified. Certain embodiments are directed to a process comprising: determining whether any nucleotide sequences that increase or potentially increase translation efficiency are not present in the elements, and incorporating such sequences into the nucleic acid composition.

An ORF sometimes is mutated or modified (for example, by point mutation, deletion mutation, insertion mutation, and the like) to alter, enhance or increase, reduce, substantially reduce or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA in cells used to prepare a cell-free extract). To determine the relative activity, the activity from the product of the mutated ORF (or cell containing it) can be compared to the activity of the product or protein encoded by the unmodified ORF (or cell containing it).

A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon sometimes is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., http address www.iupac.org/news/prize/2003/wang.pdf). Examples of unnatural amino acids are described above.

A nucleic acid composition is of any form useful for in vitro or in vivo transcription and/or translation. A nucleic acid sometimes is a plasmid, such as a supercoiled plasmid, sometimes is a linear nucleic acid (e.g., a linear nucleic acid produced by PCR or by restriction digest), sometimes is single-stranded and sometimes is double-stranded. A nucleic acid composition for transcription and/or translation can be prepared by any suitable process. A nucleic acid composition sometimes is prepared by an amplification process, such as a polymerase chain reaction (PCR) process or transcription-mediated amplification process (TMA). In TMA, two enzymes are used in an isothermal reaction to produce amplification products detected by light emission (see, e.g., Biochemistry 1996 Jun. 25; 35(25):8429-38 and http address www.devicelink.com/ivdt/archive/00/11/007.html). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

In some embodiments, a nucleic acid of other molecule described herein is isolated or purified. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. “Purified,” if a nucleic acid or protein for example, refers to a substance in a composition that contains fewer nucleic acid species or protein species other than the nucleic acid or protein of interest in comparison to the sample from which it originated. Sometimes, a protein or nucleic acid is “substantially pure,” indicating that the protein or nucleic acid represents at least 50% of protein or nucleic acid on a mass basis of the composition. Sometimes, a substantially pure protein or nucleic acid is at least 75% on a mass basis of the composition, and sometimes at least 95% on a mass basis of the composition.

Other nucleotide sequences not specifically described herein can be included in nucleic acid compositions, as selected for an application of the nucleic acid composition by the person of ordinary skill in the art.

Viral Vector-Mediated Transfer. In certain embodiments, a transgene is incorporated into a viral particle to mediate gene transfer to a cell. Typically, the virus simply will be exposed to the appropriate host cell under physiologic conditions, permitting uptake of the virus. The present methods are advantageously employed using a variety of viral vectors, as discussed below.

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5 tripartite leader (TL) sequence, which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsulation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity should be achieved.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1995; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

Other viral vectors are employed as expression constructs in the present invention. Vectors derived from viruses such as vaccinia virus (Ridgeway, 1988; Baichwal and Sugden, 1986; Coupar et al., 1988) canary pox virus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

Electroporation In certain embodiments of the present invention, a polynucleotide is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### Pharmacological Agonist

Another pharmacological approach to activation of focal adhesion signalling within muscles may involve activation by an agonist. The term “agonist” is used in the broadest sense to include any molecule that increases focal adhesion signaling or any molecule that can enhance certain FAK member activity.

Bombesin, vasopressin, endothelin, vascular endothelial growth factor, angiotensin 2, activators of integrin signaling (ie. RGD peptides), activators of G-protein signaling, reactive oxygen species, bradykinin, and Platelet-derived Growth Factor are a few examples of known pharmacological activators of the FAK signaling pathway and can be administrated along with the load-dependent stimulation of focal adhesion signalling to improve muscle function.

The compounds of the invention are administered to subjects in a biologically compatible form suitable for pharmaceutical administration in vivo. By “biologically compatible form suitable for administration in vivo” is meant a compound to be administered in which any toxic effects are outweighed by the therapeutic effects of the compound. The term subject is intended to include living organisms such as mammals. Examples of subjects include but are not limited to humans, dogs, cats, mice, rats, and species thereof. Administration of a therapeutically active amount of the therapeutic compositions of the present invention is defined as an amount effective, at dosages and for periods of time necessary to achieve the desired result. For example, a therapeutically active amount of a compound of the invention may vary according to factors such as the disease state, age, sex, and weight of the individual, and the ability of antibody to elicit a desired response in the individual. Dosage regimes may be adjusted to provide the optimum therapeutic response. For example, several divided doses may be administered daily or the dose may be proportionally reduced as indicated by the exigencies of the therapeutic situation.

As defined herein, a therapeutically effective amount of agonist (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an agonist can include a single treatment or, preferably, can include a series of treatments. It will also be appreciated that the effective dosage of in used for treatment may increase or decrease over the course of a particular treatment. Changes in dosage may result from the results of diagnostic assays as described herein.

### Routes of Administration

The active compound (i.e. an agonist) may be administered in a convenient manner such as by injection (subcutaneous, intravenous, etc.), oral administration, inhalation, transdermal application, or rectal administration. Depending on the route of administration, the active compound may be coated in a material to protect the compound from the action of enzymes, acids and other natural conditions which may inactivate the compound.

Because more load is found at the ends of muscles, administration of an active compound such as an agonist to localize in those areas is preferable.

Methods of administering a compound to an individual include providing pharmaceutically acceptable compositions. In one embodiment, pharmaceutically acceptable compositions comprise a therapeutically effective amount of one or more of the compounds described above, formulated together with one or more pharmaceutically acceptable carriers (additives) and/or diluents. In one embodiment, the active agonists may pass the blood brain barrier and may have to be chemically modified, e.g., made hydrophobic, to facilitate this or be administered directly to the muscle or via other suitable routes. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In another embodiment, the therapeutic compound is administered orally. The compounds of the invention can be formulated as pharmaceutical compositions for administration to a subject, e.g., a mammal, including a mouse or a human.

A compound of the invention can be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound of the invention by other than parenteral administration, it may be necessary to coat the compound with, or co-administer the compound with a material to prevent its inactivation. Liposomes include water-in-oil-in-water emulsions as well as conventional liposomes (Strejan et al., (1984) J. Neuroimmunol. 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

When the active compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the therapeutic treatment of individuals.

### Monitoring Enhanced In Vivo Signalling

One of skill in the art will appreciate that whatever monitoring method is used, if a quantitative result is desired, care may be taken to use a method that maintains or controls for the relative frequencies of the amplified nucleic acids to achieve quantitative amplification. Methods of “quantitative” amplification are well known to those of skill in the art. This monitoring will aid in accessing whether overexpression of a molecule within the focal adhesion signaling pathway has been achieved within a subject. Monitoring will also aid in assessing whether more or less administration of the focal adhesion molecule should be given to the subject and/or in combination with more or less muscle stimulus (see section below). For example, quantitative PCR involves simultaneously co-amplifying a known quantity of a control sequence using the same primers. This provides an internal standard that may be used to calibrate the PCR reaction. A high density array may then include probes specific to the internal standard for quantification of the amplified nucleic acid.

Another preferred internal standard is a synthetic FAK cRNA. The FAK cRNA is combined with RNA isolated from the sample according to standard techniques known to those of skilled in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known FAK RNA standard. Detailed protocols for quantitative PCR are provided in PCR Protocols, A Guide to Methods and Applications, Innis et al., Academic Press, Inc. N.Y., (1990).

In a preferred embodiment, a sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (See, e.g., Sambrook et al., “Molecular Cloning: A Laboratory Manual” (New York, Cold Spring Harbor Laboratory, 1989)._and this particular method is described in detail by Van Gelder, et al., Proc. Natl. Acad. Sci. USA, 87: 1663 1667 (1990) who demonstrate that in vitro amplification according to this method preserves the relative frequencies of the various RNA transcripts). Moreover, Eberwine et al., Proc. Natl. Acad. Sci. USA, 89: 3010 3014 provide a protocol that uses two rounds of amplification via in vitro transcription to achieve greater than 106 fold amplification of the original starting material, thereby permitting expression monitoring even where biological samples are limited.

One of skill in the art will also recognize that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The purification of a FAK polypeptide from solution can be accomplished using a variety of techniques. If the polypeptide has been synthesized such that it contains a tag such as Hexahistidine, or CBP (Stratagene), or FLAG (Eastman Kodak Co., New Haven, Conn.) or myc (Invitrogen™, Carlsbad, Calif.) at either its carboxyl or amino terminus, it may be purified in a one-step process by passing the solution through an affinity column where the column matrix has a high affinity for the tag. One of skill in the art will appreciate that essentially any vector containing another tag encoding sequence could instead, or in addition, be modified to encode a FAK binding tag of the present invention.

For example, polyhistidine binds with great affinity and specificity to nickel; thus an affinity column of nickel (such as the Qiagen™ nickel columns) could be used for purification of FAK polypeptide/polyHis. See for example, Ausubel et al., eds., Current Protocols in Molecular Biology, Section 10.11.8, John Wiley & Sons, New York (1993).

Additionally, the FAK polypeptide may be purified through use of a monoclonal antibody which is capable of specifically recognizing and binding to the FAK polypeptide.

Suitable procedures for purification thus include, without limitation, affinity chromatography, immunoaffinity chromatography, ion exchange chromatography, molecular sieve chromatography, High Performance Liquid Chromatography (HPLC), electrophoresis (including native gel electrophoresis) followed by gel elution, and preparative isoelectric focusing (“Isoprime” machine/technique, Hoefer Scientific, San Francisco, Calif.). In some cases, two or more purification techniques may be combined to achieve increased purity.

Within laboratory animal models, enhanced in vivo FAK signalling via gene transfer may be analyzed with immunoblotting and immunofluorescence, details are described further herein.

### C. Muscle Stimulation

One of skill in the art may use any technique to achieve the desired results of muscle stimulation. Known exercise techniques are conventionally classified as isometric, isotonic, and isokinetic. All of these techniques except isometric utilize motion of the limb for strengthening or treating an injured muscle and all of the techniques can have corresponding exercise equipment associated with them in order to provide more load. For example, muscles that are stimulated may be flexed, extended or electrically stimulated.

Isometric exercise is a strength-training exercise, where muscles contract but the joints do not move and muscle fibers maintain a constant length. The exercises are typically performed against an immovable surface for example, pressing the palm of a hand against a wall. Isometric training is effective for developing total strength of a particular muscle or group of muscles. This is sometimes used for rehabilitation because the exact area of muscle weakness can be isolated and strengthening exercises can be administered at the proper joint angle. Isometric strength training is not ideal for sports training, but it has many useful purposes. This kind of training can provide a relatively quick and convenient method for overloading and strengthening muscles without any special equipment and with less chance of injury.

In isotonic exercise, a body part is moved and the muscle shortens or lengthens. Although sit-ups, push-ups and pull-ups are isotonic, lifting free weights, like dumbbells and barbells, is considered the classic form of isotonic exercise.

Isokinetic exercise is performed with a specialized apparatus that provides variable resistance to a movement, so that no matter how much effort is exerted, the movement takes place at a constant speed. Such exercise is used to test and improve muscular strength and endurance, especially after injury.

Flexibility exercises use gentle, stretching movements to increase the length of the muscles and the effective range of motion in joints. They may consist of a series of specific stretching exercises, or be part of a larger exercise program such as yoga or dance classes. Because one of the main goals of stretching is to lengthen the connective tissue surrounding the muscle fibers, flexibility exercises should be done after the muscles have been warmed up by a few minutes of aerobic activity. Although flexibility exercises do not offer the dramatic overall benefits of aerobic or resistance exercise, regular stretching (several times a week) can be an important way to maintain a body's mobility and freedom of movement, particularly as one gets older. Stretching exercises can also improve posture and are an essential part of effective long-term treatment for strained or chronical muscle injuries. Flexibility exercises can be an important part of an injury-prevention or rehabilitation program when chronically tight muscle groups contribute to the problem.

Electrical muscle stimulation (EMS) is the concept whereby electric impulses are used to contract muscles. EMS has been used in the field of medicine as therapy for muscle atrophy, as well as in many other conditions. Electrical muscle stimulation usually is localized to stimulate a part of the body. For this purpose, an electronic device is used, whereby small electrodes are directly placed onto the body area(s) that needs to be stimulated. A slow tension is then put on the wires and muscle stimulation is performed. Changes in the voltage can stimulate different pressure on the muscles, for creating various effects. The electrical impulses provide a strong stimulation for the muscles. Low voltage is typically used on smaller, involuntary muscle groups, which cannot be stimulated in other ways. For example, low voltage can be used to stimulate the brain, which can then start sending impulses through the involuntary muscles, thus stimulating them as well.

### D. Treatment Methods, Dosages, and Cominbation Therapies Responsiveness to Treatment

Monitoring the influence of agonists or gene therapy on the overexpression of FAK signalling along with load-dependent stimulation can be applied during clinical trials, muscle therapy and basic drug screening.

One of ordinary skill in the art may identify a variety of methods to monitor responsiveness to treatment. Any molecule that can assess enhancement to focal adhesion signaling may be monitored for determining whether the subject is responsive to treatment. For example, the effectiveness of an agonist or gene therapy along with load-dependent stimulation determined by a screening assay as described herein to overexpress FAK signalling, can be monitored in clinical trials of subjects exhibiting increased ribosomal S6 kinase levels. In such clinical trials, phosphorylation patterns of S6 kinase that have been implicated in FAK signalling can be used as a “read out.” Any molecule that can assess enhancement to focal adhesion signaling may be monitored for determining whether the subject is responsive to treatment and any screening method may be used to measure any of these changes such as in levels, activity, localization, binding and the like of such molecules. For instance, levels of DNA, RNA, protein and the like, or phosphorylation levels, or binding to particular molecules or not binding to particular molecules, localization variation and the like are examples of changes to monitor. Any molecule may include focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

For example, and not by way of limitation, S6 kinase phosphorylation that is increased in cells by treatment with an agonist or gene therapy along with load-dependent stimulation which increases S6 kinase phosphorylation (e.g., identified in a screening assay as described herein) can be identified. Thus, to study the effect of agonists or gene therapy along with load-dependent stimulation on muscle improvement, for example, in a clinical trial, cells can be isolated and protein prepared and analyzed for the levels of S6 kinase phosphorylation. The levels of S6 kinase phosphorylation may be analyzed by measuring the amount of phosphorylation by one of the methods as described herein. In this way, the phosphorylation pattern can serve as a marker, indicative of the physiological response of the cells to the agonist or gene therapy along with load-dependent stimulation. Accordingly, this response state may be determined before, and at various points during treatment of the individual with the agonist or gene therapy along with load-dependent stimulation. Other useful markers of FAK signalling are described further herein.

In one embodiment, the present invention provides a method for monitoring the effectiveness of treatment of a subject with an agonist or gene therapy along with load-dependent stimulation including the steps of (i) obtaining a pre-administration sample from a subject prior to administration of the agonist or gene therapy along with load-dependent stimulation; (ii) detecting the level of S6 kinase phosphorylation in the preadministration sample; (iii) obtaining one or more post-administration samples from the subject; (iv) detecting the level of S6 kinase phosphorylation in the post-administration samples; (v) comparing the level of S6 kinase phosphorylation in the pre-administration sample with the S6 kinase phosphorylation in the post administration sample or samples; and (vi) altering the administration of the agonist or gene therapy and/or load-dependent stimulation to the subject accordingly. For example, increased administration of the agonist/gene therapy and/or load-dependent stimulus may be desirable to increase S6 kinase phosphorylation to higher levels than detected, i.e., to increase the effectiveness of the agonist/gene therapy and/or load-dependent stimulus. Alternatively, decreased administration of the agonist/gene therapy and/or load-dependent stimulus may be desirable to decrease S6 kinase phosphorylation to lower levels than detected, i.e., to decrease the effectiveness of the agonist/gene transfer and/or load-dependent stimulus. According to such an embodiment, S6 kinase phosphorylation may be used as an indicator of the effectiveness of an agonist/gene transfer and/or load-dependent stimulus, even in the absence of an observable phenotypic response.

An exemplary method for detecting phosphorylation (e.g., tau phosphorylation, S6 kinase phosphorylation and the like) in a biological sample involves obtaining a biological sample from a test subject and contacting the biological sample with a compound or an agent capable of detecting phosphorylated peptide such that the presence of phosphorylated peptide is detected in the biological sample. As used herein, the term “peptide” refers to polypeptides comprising two or more amino acid residues, full length protein sequences, fragments of protein sequences and the like. In one aspect, an agent for detecting phosphorylated peptide is an antibody capable of binding to the peptide (e.g., binding to S6 kinase), such as an antibody with a detectable label. In one aspect, the antibody only binds to hyperphosphorylated S6 kinase and/or S6 kinase phosphorylated at threonine 421. Antibodies which bind only to phosphorylated S6 kinase are described herein. Antibodies can be polyclonal, or in another aspect, monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect phosphorylated peptide (e.g., S6 kinase) in a biological sample in vitro as well as in vivo. For example, in vitro techniques for detection of phosphorylated S6 kinase include enzyme linked immunosorbent assays (ELISAs), Western blots, immunoprecipitations and immunofluorescence. In vitro techniques for detection of phosphorylated S6 kinase include introducing into a subject a labeled anti-S6 kinase antibody. For example, the antibody can be labeled with a radioactive marker whose presence and location in a subject can be detected by standard imaging techniques.

In another embodiment, the methods of the invention further involve obtaining a control biological sample from a control subject, contacting the control sample with a compound or agent capable of detecting phosphorylated peptide (e.g., S6 kinase), such that the presence of phosphorylated peptide is detected in the biological sample, and comparing the peptide phosphorylation in the control sample with the presence of increased peptide phosphorylation in the test sample. As described herein, one or more database can also be used as the control.

The diagnostic methods described herein can furthermore be utilized to identify the responsiveness to treatment of a subject with muscle degenerating disease, for example, by determining the absence or decreased amount of peptide (i.e., S6 kinase) phosphorylation. As used herein, the term “decreased” includes peptide phosphorylation which is decreased relative to either previously samples of the subject's peptide phosphorylation levels or the wildtype peptide phosphorylation indicative of an individual not suffering from a muscle degenerative disease (i.e., a healthy individual). Responsiveness to treatment include hyperphosphorylation and/or phosphorylation of serine 411 and/or the dual site threonine 421/serine 424 of S6 kinase, as well as phosphorylated S6 kinase expression or activity which does not follow the wildtype developmental pattern of expression or the subcellular pattern of expression.

Furthermore, the therapeutic assays described herein can be used to determine whether a subject can be administered an agonist/gene therapy and/or load-dependent stimulus with to treat muscle disease. Thus, the present invention provides methods for determining whether a subject can be effectively treated with an agonist/gene therapy and/or load-dependent stimulus for muscle disease associated with increased S6 kinase phosphorylation in which a test sample is obtained and S6 kinase phosphorylation is detected.

### E. Assessing Improved Muscle Function

Improved muscle function can be determined by electrophysiological and mechanophysiological studies. In such studies, only one muscle, such as the soleus, rather than all major muscle groups should be tested. The soleus, containing many red fibers that are slow twitching, is unique and different from other muscles in the body that are composed of fast twitching fibers. In humans and some other mammals, the soleus is a powerful muscle in the back part of the lower leg (the calf). It runs from just below the knee to the heel, and is involved in standing and walking. It is closely connected to the gastrocnemius muscle and some anatomists consider them to be a single muscle, the triceps surae. The soleus is located in the superficial posterior compartment of the leg. Not all mammals have a soleus muscle, for example dogs lack soleus muscles. The action of the calf muscles, including the soleus, is to plantar flex the foot (that is, they increase the angle between the foot and the leg). They are powerful muscles and are vital in walking, running, and dancing. The soleus specifically plays an important role in standing; if not for its constant pull, the body would fall forward. Also, in upright posture, it is responsible for pumping venous blood back into the heart from the periphery.

Muscle dystrophic characteristics in mice include muscle fiber splitting, central nucleation, phagocytic necrosis, variation in fiber shape and size, and increase in intercellular connective tissues. Within a specified amount of time after treatment, behavior and movement may be assessed. For example, two to four months after treatment, mice with damaged muscles can be tested for such behavioral improvement and their locomotive patterns in comparison with those of the wildtype mice. Sporadic flexion and flaccid extension of their hindlimbs can be monitored as well as their ability to use their hindlimbs and toes. Certain exercises can be performed such as testing whether they can balance themselves on a glass rod by using their hindlimb muscles or their ability to run. The occasional walking with duck feet, or splayed toes is a normal phenotype. Muscle bulk can be assessed in both legs and in the chest. Normal littermates treated similarly have been shown to be hyperactive, displaying enlarged leg and intercoastal muscles, but were otherwise normal.

### A. EXAMPLES

The examples set forth below illustrate but do not limit the invention. These experiments tested the suitability of skeletal muscle to elucidate the signaling processes governing the major control of protein translation by muscle loading. These studies explored the possible functional coupling between the activation of a key player of protein translation in striated muscle, the 70 kDa ribosomal protein S6 kinase (p70S6K) and the integrin-associated focal adhesion kinase (FAK). This focus was motivated by observations indicating that mechano-signaling via FAK to the serine/threonine p70S6K might be the missing molecular connection in the important control of muscle protein synthesis by mechanical factors. This is indicated by the role of FAK as a sarcolemmal mechano-transducer in striated muscle, the positioning of FAK upstream of p70S6K activation in different cell cultures and the association of the phosphotransfer activity and phosphorylation of both kinases with load-dependent increases in muscle mass. Results demonstrate that muscle fiber-targeted FAK overexpression in combination with the mechanical stimulus of reloading after prolonged muscle unloading would enhance p70S6K-mediated translation control in tibialis anterior (TA) muscle. Also elucidated is the time course and relationship of the early FAK activation to the putative downstream phosphorylation of p70S6K and “S6K-independent” translation factors at important regulatory sites after the mechanical stimuli.

### Example 1

### Methods and Materials for Examples 1-5

Cytomegalovirus (CMV) promoter-driven expression plasmid pCMV encoding chicken FAK gene (pCMV-FAK) or an empty plasmid (pCMV) were isolated under endotoxin-free conditions at Plasmid Factory GmbH (Bielefeld, Germany, www.plasmidfactory.de). Fluorescent-compatible mounting medium was from DAKO (DAKO, Carpinteria, Calif., USA). Bicinchoninic acid assay reagents and protein A Sepharose were from Sigma (Sigma-Aldrich, St. Louis, Mo., USA). The deployed antibodies against the signaling molecules in focus and the verified phosphorylation sites involved in their regulation are summarized in Table 1. Peroxidase-conjugated secondary antibodies goat anti-rabbit IgG and goat anti-mouse whole IgG were obtained from ICN Biomedicals GMBH (Germany). Alexa488-conjugated antirabbit IgG antibody was from Invitrogen (Invitrogen, Basel, Switzerland). Super Signal West Femto Kit and Kodak XAR5 films were from Pierce (Perbio Science, Lausanne, Switzerland) and Sigma (Buchs, Switzerland), respectively.

TABLE 1
Name, function, epitope and source of all primary antibodies used to identify FAK dependentsignaling in this study.

| Antibody | Function | Epitope | Source |
|---|---|---|---|
| FAK | Tyrosine kinase | N-terminal (A-17) | Santa Cruz, CA, USA |
| FAK | Tyrosine kinase | C-terminal | Ziemiecki [Flueck et al. 1999] |
| pFAK-Y397 | Major auto-phosphorylation | Phospho-Tyrosine 397 (Y397) | Santa Cruz, CA USA BioSource |
| | and activation site | | Europe (Nivelles, Belgium) |
| p70S6K | Key regulator of mRNA | P70S6K(C-18) | Santa Cruz, CA USA |
| | translation | | |
| p-p70S6K-S411 | Pre-activation of p70S6K | Phospho-Serine 411 (S411) | Santa Cruz, CA, USA |
| p-p70S6K-T421/S424 | Pre-activation of p70S6K (a | Phospho-Threonine 421 | Santa Cruz, CA, USA |
| | Serine/Threonine kinase) | and Serine 424 (T421/S424 | |
| p-3IF2alpha-S52 | Regulation of translation | Phospho-Serine 52 (Ser52) | BioSource Europe, Nivelles, Belgium |
| | initiation | | |
| p-4E-BP1-T37/46 | Regulation of translation | Phospho-Threonine 37 | Cell Signaling Technology, |
| | initiation | and 46 (T37/46) | Danvers, MA, USA |

Animals. The animal protocol was approved by the Animal Protection Commission of the Canton Berne, Switzerland. The 6 month old male mice 129/SVEV weighed 35.4±0.7 g (n=17) before the intervention. They were housed individually in a temperature-controlled room (21° C.) with a 12:12 h light-dark cycle. Animals were allowed food and water ad libitum.

Gene electro transfer Intramuscular gene transfer was achieved via injection of plasmid DNA and subsequent electrotransfer with modifications as previously described. The mice were individually anesthetized with isoflurane and the lower limbs were shaved. 25 μg of expression plasmid in 25 μl physiological saline solution (0.9% NaCl) was injected with a sterile 100 μl syringe into the tibialis anterior (TA) muscle. After 5 minutes of incubation, electric pulses (4 trains of 100 pulses of 100 μsec each at 50 mA at 4 different locations) were delivered using the GET42 pulser with needle electrodes (E.I.P. Electronique et Informatique du Pilat, Jonzieux, France). Typically, mice recovered rapidly from this procedure and begin to move freely 2 hours after the intervention.

Modulation of muscle loading. The animals were subjected to either of five different loading conditions basically as previously described. Two days after the gene electrotransfer, animals were subjected to 7 days of hindlimb unloading (HU) by tail suspension or kept as cage controls (CC). Subsequently, a set of suspended animals was subjected to reloading for 1 hour (R1), 6 hours (R6) or 24 hours (R24). Cage activity was encouraged in the first hour of reloading by tipping the finger into the suspension cage. TA muscles were harvested from anesthetized animals, rapidly weighed, frozen in nitrogen-cooled isopentane and stored at −70° C. for subsequent analysis.

Confocal microscopy. The subcellular localization of FAK was detected on cryosections as previously described, but with the modification that fluorescent-labelled secondary antibodies were used. The deployed primary FAK antibody A-17 was applied at a 1:100 dilution in 0.3% BSA/PBS, reacted with 200-fold diluted Alexa488-conjugated anti-rabbit IgG and embedded in fluorescent-compatible mounting medium. Fluoroscence and digital phase contrasts were analyzed with a Leica TCS SP5 confocal microscope (Leica Microsystem CMS, Wetzlar, Germany).

Immunoblotting. Protein homogenate was prepared by minzing frozen mouse TA muscles for 20 seconds on ice with a Polytron homogenizer (Kinematica) in RIPA buffer. The homogenate was incubated for 20 minutes on a shaker (1000 rpm, 4 degree Celsius) and centrifuged for 5 minutes (10000 g, 4 degree Celsius). Soluble proteins were recovered in the supernatant and protein concentration determined with the bicinchoninic acid assay. SDS-PAGE, Western blotting and immunodetection were performed with specific antibodies (see Table 1) as previously described. Standardized amounts of protein (20 μg) were loaded per well. Western transfer efficiency and equal loading was controlled by visual inspection of the Ponceau S stained membrane. Signal was recorded with enhanced chemiluminescence by using the Super Signal West Femto Kit and Kodak XAR5 films. The signal intensity of the specific protein band was determined using the line and band density mode in the Quantity One 1-D analysis software 4.6.1 (Bio-Rad, Life Science Research, Hercules, Calif., USA). Immunoprecipitation was performed with 1 mg protein in 750 μl RIPA buffer. Therefore, 1 μl pFAK serum from BioSource and 10 μl p-FAK from Santa Cruz were combined and incubated with 5 mg Protein A Sepharose (Sigma) with shaking at 4 degrees Celsius for 2 hours. After incubation of antibodies with the protein sample over night, the immunocomplexes were precipitated by centrifugation for 10 minutes (10000 g, 4 degree Celcius), washed twice in 1 ml RIPA and resuspended in SDS loading buffer for separation by 7.5% SDS-PAGE.

In vitro S6 Kinase activity assay. Phosphotransfer activity of p70S6K was evaluated in vitro. S6 Kinase phosphorylation was initiated by the addition of 75 μg protein homogenate to 45 μl preheated phosphorylation mixture including S6K substrate peptide (RRRLRRLRA) at 30° C. basically as described. The reaction was stopped after 5 minutes by spotting 20 μl on a Whatman P81 filter and by washing in 75 mM H3PO4 and Acetone. Quantification of incorporated 32P was performed by liquid scintillation counting. To technical replicas were measured from each sample.

Statistics. Due to the large total sample number, not all samples could be analyzed in one assay. In order to minimize the influence of inter-assay error in the quantitative analysis of molecular parameters, a paired analytic design was employed: Muscle samples from contralateral transfection pairs were analyzed in the same assay. For immunoblotting samples from contralateral muscle pairs (i.e. pCMV-FAK and pCMV-empty-transfected TA) were separated in adjacent lanes of the SDS-PAGE gel. A reference sample was run in all gels. Data were related to the reference sample, normalized to the mean of signal for empty transfection in cage controls on the respective gel, biological replica from different gels were pooled.

Statistical analysis was carried out with Statistica 6.1 (StatSoft Europe, Hamburg, Germany). The expression and phosphorylation level in pCMV-transfected muscles, as well as body and muscle weight, were evaluated with a one-factor analysis of variance (ANOVA).

Differences between pCMV-FAK and pCMV-transfected left and right muscle pairs were analyzed with a Friedman-ANOVA with repeated measurements. This was based on the experimental design where protein measures in muscle pairs reflected biological repetitions of electrotransfer and loading stimulus but where the plasmid identify was modulated. Subsequently, a Fisher post hoc test was carried out to localize the effect. Linear regression analysis was carried out with Pearson correlation. A p-value of 0.05 was selected as the significance level for all tests. Values are given as means±standard error (SE).

### Example 2

### Effect of Muscle Loading on FAK Overexpression and Activity

Interaction of electrotransfer and muscle loading. TA muscle pairs of adult mice were subjected to gene electrotransfer. Right TA was transfected with constitutive-active plasmid for chicken FAK, i.e. pCMV-FAK. The contralateral TA was transfected with pCMV-empty plasmid. Subsequently, animals were subjected to 7 days of unloading followed by a course of reloading for 1 hour, 6 hours or 24 hours or housed under normal cage activity for the same total time duration. Body weight was reduced with unloading and returned to cage control level after 1 day of reloading. The characterization of electro-transferred TA muscle also confirmed the expected drop in TA wet weight with 7 days of unloading and showed return of muscle weight to basal levels within one day of reloading without reaching the level of significance among the different time points (Table 2).

TABLE 2
Body weight and tibialis anterior muscle weight of all mice used
in this study. Effect of time was analyzed with a one-factor
ANOVA and the effect was localized with a Fisher-
LSD post-hoc test (*). The level of significance was p < 0.05.

| | HU7 | HU7R1 | HU7R6 | HU7R24 | CC | p-value |
|---|---|---|---|---|---|---|
| number | n = 2 | n = 3 | n = 3 | n = 4 | n = 5 | |
| Body weight [g] | 30.5 ± 1.1 | 28.9 ± 0.6 | 29.8 ± 1.2 | 34.4 ± 0.1 | 38.2 ± 0.8 | 0.002 |
| left TA weight | 34.0 ± 4.0 | 37.3 ± 0.9 | 35.3 ± 1.5 | 40.3 ± 4.7 | 46.0 ± 2.0 | 0.658 |
| [mg] | | | | | | |
| right TA | 38.0 ± 1.0 | 39.7 ± 2.8 | 33.0 ± 2.1 | 44.7 ± 2.0 | 45.0 ± 1.5 | 0.049 |
| weight [mg] | | | | | | |

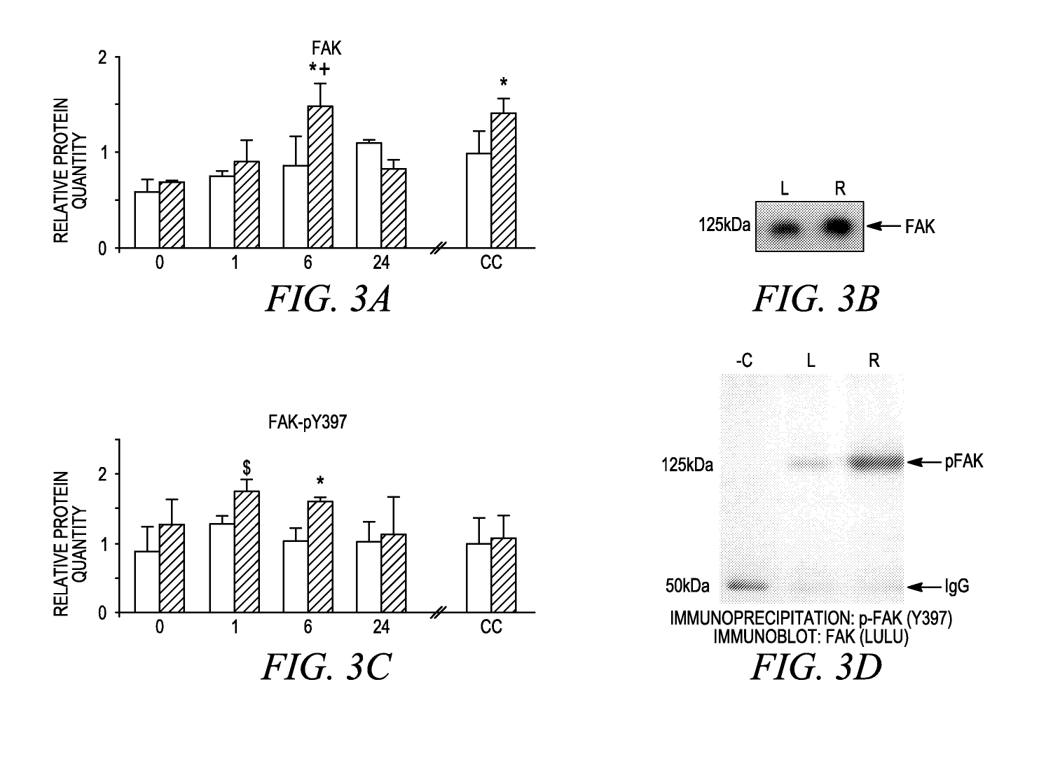

FAK overexpression. The expression level and localization of FAK protein in transfected TA muscle was analyzed with immunoblotting and immunofluorescence. FIG. 2 depicts FAK-immunostaining in a pCMV-FAK overexpressing cryosection. Within the FAK positive fiber most of the exogenous FAK is located near the plasma membrane and only some in the cytoplasm. The majority of FAK localized to the sarcolemma and punctuate staining was observed in the sarcoplasm. A proportion of fibers in empty transfected muscle demonstrated FAK-immunostaining but staining intensity was more pronounced in pCMV-FAK transfected muscle. Gene electrotransfer also caused muscle fiber damage in the transfected region, which was comparable for both conditions of transfection (not shown). Qualitative immunoblotting identified a 1.6-fold increase in FAK protein levels in cage controls 9 days after electrotransfer (FIG. 3A). The mouse FAK isoform was detected as a single band at 125 kDa in empty and chicken FAK transfected muscles (FIG. 3B).

Effect of muscle loading on FAK overexpression and activity. The difference in FAK expression was load-dependent. FAK overexpression in pCMV-FAK transfected muscle was lost with 7 days of unloading but was reestablished within 6 hours of reloading (FIG. 3A). This increase in FAK protein with reloading was preceded by enhanced phosphorylation of FAK on the major activation site Tyr397 after 1 hour of reloading (FIGS. 3 C and D). This tyrosine phosphorylation with reloading was transient and lost after 24 hours and correlated significantly with FAK protein level (r=0.45). A: FAK protein levels are elevated in cage control and after 6 hours of reloading; white bars: pCMV-empty plasmid in left TA muscle; black bars: pCMV-FAK plasmid in TA right muscle; CC: cage control, 9 days of overexpression; 0: 7 days of hindlimb unloading and no reloading; 1: 7 days unloading and 1 hour of reloading; 6: 6 hours of reloading, 24: 24 hours of reloading; Friedman-ANOVA: *, p<0.05 versus contralateral control; +, p<0.05 versus time point 0. B: Representative Western Blot experiment of cage control muscle 9 days after gene electrotransfer; L: pCMV-empty of CC sample; R: pCMV-FAK of CC sample. C: activation of FAK via phosphorylation on Tyr397 after 1 and 6 hours of reloading; Friedman-ANOVA: *, p<0.05 versus contralateral control; 0.05<p<0.10 versus contralateral control. D: Immunoprecipitation was performed with pFAK (Y397) and immunoblotting with FAK (Lulu) antibody. Note that in the negative control more IgG protein was added to the sample and that this did not precipitate any FAK protein from the muscle homogenate; −C: negative control, i.e. immunoprecipitation without p-FAK antibody; L: pCMV-empty of time point 6; R: pCMV-FAK of time point 6; IgG: Immunoglobulin gamma heavy chain.

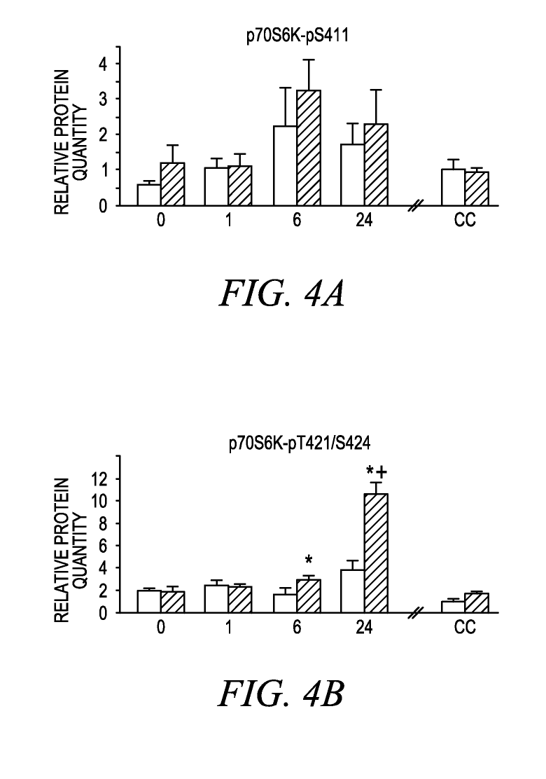

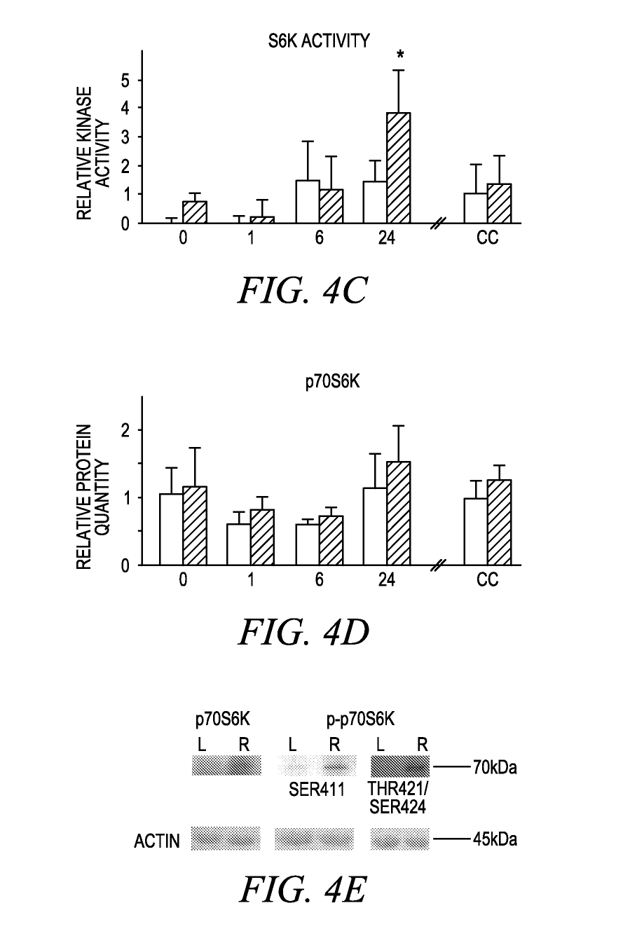

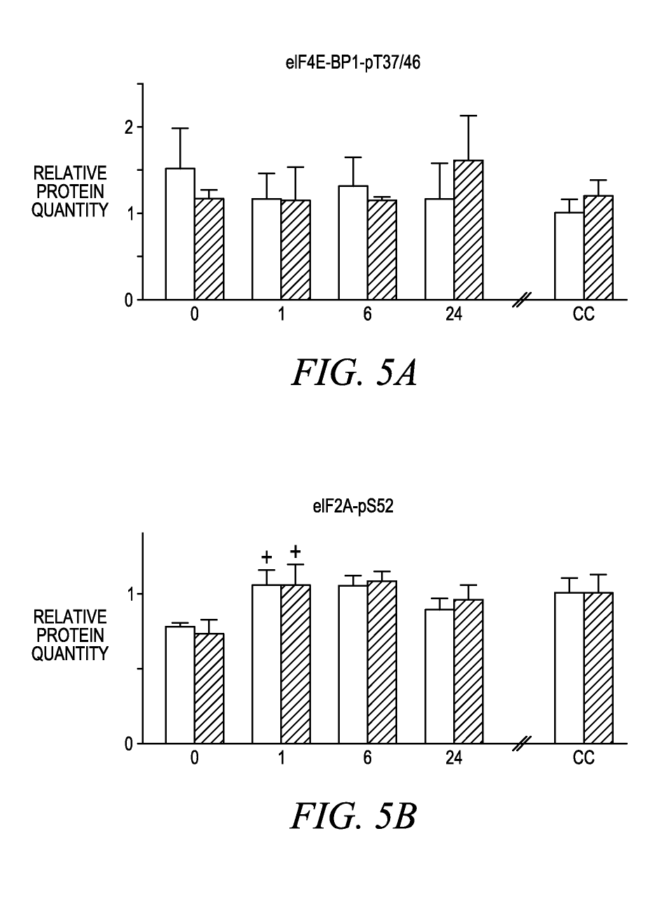

Load-dependent p70 S6K-signaling in FAK-transfected muscle. We tested whether the added mechanical stress of reloading in combination with FAK overexpression would activate p70S6K in muscle which was deconditioned by unloading. Unloading did not bring about significant differences in p70S6K amount, phosphorylation and phosphotransfer activity between FAK- and empty-transfected TA muscles (FIG. 4A-D). (A) and on Thr421/Ser424 (B), S6 Kinase in vitro kinase activity (C) and p70S6 Kinase protein level (D). E: Representative experiments of p70S6K Western Blots shown in A-D and corresponding Actin protein staining; white bars: pCMV-empty in left (L) muscle; black bars: pCMV-FAK in right (R) muscle. Friedman-ANOVA: *, p<0.05 versus contralateral control; +, p<0.05 time point 0. Subsequent reloading altered the phosphorylation status of p70S6K in deconditioned TA muscle both qualitatively and quantitatively: 6 hours after the first ground contact of hindlimbs, p70S6K was increasingly phosphorylated on the dual site Thr421/Ser424 in FAK transfected muscle versus their contralateral controls, which peaked after 24 hours of reloading (FIGS. 4A and B). Phosphorylation on Ser411 showed a near trend for FAK transfection mediated elevation 6 hours after reloading. Functionally important regulation of p70S6K by loading was emphasized by a significant 3.8-fold enhanced p70S6 K phosphotransfer activity in FAK-versus empty-transfected muscle after 24 hours of reloading (FIG. 4 C). p70S6K protein levels were not affected by reloading of FAK overexpression (FIG. 4D). Phosphotransfer activity and p70S6K phosphorylation status of the verified sites during the reloading response were significantly correlated for both conditions of transfection (pS411: r=0.73; pT421/S424: r=0.60). The verification of two key translation initiation factors identified no significant effect of FAK-transfection. Neither phosphorylation on the key regulatory sites Thr37/Thr46 in the eukaryotic translation initiation factor 4E binding protein 1 (eIF4E-BP1) nor the activating site Ser52 in the eukaryotic initiation factor 2 alpha (eIF2A) was significantly affected by the introduction of FAK (FIGS. 5 A and B). (A) and eIF2A on Ser52 (B). white bars: pCMV in left muscle, black bars: pCMV-FAK in right muscle. Friedman-ANOVA: +, p<0.05 versus time point 0. Reloading per se increased however Ser52 phosphorylation of eIF2A after one hour of reloading in both transfection conditions. The total protein content of eIF2 was unchanged (data not shown). eIF2A pS52 was negatively correlated to p70S6K pT421/S424 and eIF4E-BP1 pT37/46 phosphorylation.

### Example 3

### Test System and Technical Considerations

Electrotransfer of naked DNA is a classic technique for transfection of suspended cells in culture. After ground breaking work a decade ago demonstrating the importance of technical parameters in electropulsing for the transfection rate, gene electrotransfer is now increasingly applied at the organ level. This technique is particularly effective in skeletal muscle and gene electrotransfer of exogenous promoter reporter constructs has recently been applied to the study of gene regulation in this tissue. Our current investigation indicates that myocellular overproduction of an exogenous tyrosine kinase (i.e. chicken FAK) via electro-assisted transfection in combination with a paired design also develops the nominal resolution to directly identify endogenous biochemical signaling during physiologically-induced adaptations. In the following, pertinent technical and biological aspects of the investigated phenomenon of mechano-dependent translation control are addressed.

Technical considerations on the deployed experimental approach indicate the important contribution of biological variables other than FAK and muscle loading to the measured FAK mediated signaling. Foremost this is presented by the reported damage response of transfected muscle portions by the selected methodology of gene transfer. The subsequently induced biological process of muscle regeneration likely influenced the observable FAK dependent signaling response since sarcolemmal levels of FAK are elevated during fiber renewal. This possibility is indicated by the detectable sarcolemmal FAK staining in a proportion of fibers of empty-transfected TA muscle (see FIG. 2B) which is expected to show a low level of FAK staining. In this context it is important to note that massive fiber damage and regeneration was the major bias associated with somatic muscle transgenesis before the advent of the convenient electro-assisted gene transfer. In our setting this bias was controlled by comparing the net effect of transfection in contralateral muscle pairs between FAK-producing and empty expression plasmid. This “subtraction” allowed the identification of statistically significant effects of FAK-transfection on p70S6K signalling in transfected TA muscle which were mechano-modulated throughout time. This is considerable taking into account the relative low percentage of muscle fiber transfection, the moderate responsiveness of TA muscle to hindlimb unloading compared to other leg muscle groups and the restrictions imposed by the relatively low number of animals per experimental group. This highlights the resolution power of our approach for exposing muscle signaling.

With regard to the specificity of our somatic transfection experiments we identified that the exogenously introduced chicken FAK did not distinguish to the endogenous mouse isoform in the soluble fraction based upon size (see FIG. 3 B). This observation corresponds to previous molecular work concluding on a highly conserved amino acid sequence between FAK homologues from the two species. It indicates that the addition of an introduced chicken FAK homologue does not visibly alter the gel migration of unphosphorylated and phosphorylated FAK isoforms.

### Example 4

### Specificity of FAK-Mediated Mechano-Signaling

The measured control of FAK protein and tyrosine phosphorylation levels in transfected muscles implies an important physiological modulation of FAK function by muscle loading. This regulation of Tyr397 phosphorylation and amount of FAK between FAK-transfected and empty-transfected muscle differed with regard to the “effective” time of loading. For instance, total level of Tyr397 phosphorylation was transiently enhanced by reloading without a change in FAK protein. In cage controls, no difference in Tyr397 phosphorylation was however visible between FAK-transfected and empty-transfected TA muscle when total FAK protein levels were elevated. We suggest that the elevated FAK activation within the first hours of reloading reflects the possibly higher mechanical impact of normal cage activity on TA muscle after a period of unloading than in cage controls. This observation also hints for an elevated potential of FAK-mediated mechano-transduction after muscle deconditioning. The absence of differences in FAK protein between both FAK- and control-transfected muscles indicates the implication of a process upstream of FAK tyrosine phosphorylation to enhance mechano-sensitivity.

Downstream mechano-signaling of FAK. The molecular measures demonstrate that the experimental enhancement of FAK-signaling transduces a mechanically-imposed stimulus to the delayed activation of p70S6K-signaling. These temporal and mechanistic relationships between FAK and p70S6K-phosphorylation and phosphotransfer activity establish that an important load-modulated signaling pathway of translation control in fully-differentiated muscle is under control of FAK. The measured phosphorylation of p70S6K allows important regulatory conclusions on the pathway connecting FAK to p70S6K activation. The FAK-modulated phosphorylation on Ser411 and the dual phosphorylation Thr421 and Ser424 of p70S6K points to the involvement of serine/threonine kinases since FAK activity explicitly targets tyrosine residues. The measured p70S6K phosphorylation sites are targeted by numerous kinases, including PI3K, Akt, PDK1, mTOR and PKC, which could mediate the identified connections of FAK and p70S6K activation in vivo.

### Example 5

### P70S6K-Mediated Translation Control

Our observations in intact muscle recapitulate the reported role of p70S6K phosphorylation for protein synthesis in vivo. For instance p70S6K phosphorylation on Thr421 and Ser424 has been shown to correlate with gains in muscle mass in different animal models (i.e. stretch and resistance exercise) for muscle hypertrophy. The assessed sites control biochemical function of p70S6K in vitro and their enhanced phosphorylation is believed to stimulate protein synthesis in culture. Phosphorylation of p70S6K on Ser411 and the tandem Thr421/Ser424 relieves the phospho-transfer activity from autoinhibition prior to a full activation of the enzyme. The correlation of phosphorylation at the latter tandem sites in FAK-transfected muscles supports the notion of a functional implication of FAK stimulated p70S6K activation in translation control in vivo. The findings corroborate earlier suggestions on a role of FAK in protein synthesis and cell size regulation. These results now imply a functional contribution of FAK in modulating the load-induced hypertrophic response of muscle due to p70S6K-mediated induction of protein translation.

### Example 6

### Material and Methods for Examples 6-8

Reagents. pCMV-expression plasmids with constitutively active cytomegalo virus (CMV) promoters were used. pCMV-b-galactosidase reporter plasmid was from BD Clontech (Basel). Plasmids for overexpression of chicken FAK (pCMV-FAK) and FRNK homologues (pCMV-FRNK) were received as a gift from Tony Parsons. The amino acid sequences are highly conserved between the chicken and rat FAK homologue (92%) with all major regulatory sites being present. Empty pCMV plasmid was used as a control. Plasmids were sent out to plasmidfactory (Bielefeld, Germany, www.plasmidfactory.de) for propagation in bacteria and isolation of endogen-free DNA. Polyclonal rabbit antibodies A-17 against the FAK N-terminus was from Santa Cruz Biotechnology. FAK-pY397-specific antibody was from. C-terminal FAK serum was a gift of Dr. Andrew Ziemiecki (University of Bern, Bern, Switzerland) and has been characterized previously (Fluck et al., 1999). Monoclonal antibodies against mitochondrial cytochrome C subunits I and IV were from Molecular probes (Eugene, Oreg.). Type I and II myosin heavy chain and tenascin-C antibodies were applied as described previously (Fluck et al., 2005). Horse radish-peroxidase-conjugated antirabbit and anti-mouse secondary antibodies (Cappel Inc.) were used at dilution 1:5,000.

Animals-3-month old, female pathogen-free Wistar rats (Charles River Laboratory, L'Arbresles, France) were delivered to Lyon or Berne. After a first phase of recovery from the travelling, animals were acclimatized for housing in single cages prior to entry in the specific experiments.

Somatic transgenesis-Gene electrotransfer was basically carried out as described (Durieux et al., 2002) and optimized for soleus muscle. Animals were anesthetized, the skin of the lower leg was shaved and a medial incision was made. Then the connective tissue sheet between the gastrocnemius and tibialis anterior muscle was split and the soleus muscle was surgically exposed with the help of a threat and forceps. Endotoxin-free plasmid (see ‘Experimental design’) was injected in the belly portion of soleus muscle. After 5 minutes incubation, 3 trains of 80 pulses of 100 μsec each at 100 mA were applied with the GET42 generator (E.I.P. Electronique et Informatique du Pilat, Jonzieux, France). Needle electrodes were used with a 4-mm gap to deliver electrical stimulation to the injected muscle portion. After gene electrotransfer, the skin and fascia were closed with sutures and animals transferred in the single cages for the rest of the experiment. Rats recovered rapidly from this procedure and begin to walk the next day.

Muscle loading-Loading of rat soleus muscle was modulated by hindlimb unloading-reloading and tenotomy. Unloading and corresponding gene electrotransfer experiments were performed in the animal facilities of the Universite Claude Bernard Lyon I in Villeurbanne, France. In these experiments, anaesthesia was achieved via intraperitoneal injection of sodium pentobarbital (60 mg/kg body weight, Sanofi, France). For the tenotomy experiments, gene electrotransfer was carried out at the Institute of Anatomy of the University of Berne under 2% isoflurane anesthesia. The freshly transfected animals were transported to Pavia for tenotomy (Italy). Experiments were performed with permission of the local Animal Care Committee under compliance with the newest guiding principles for animal research.

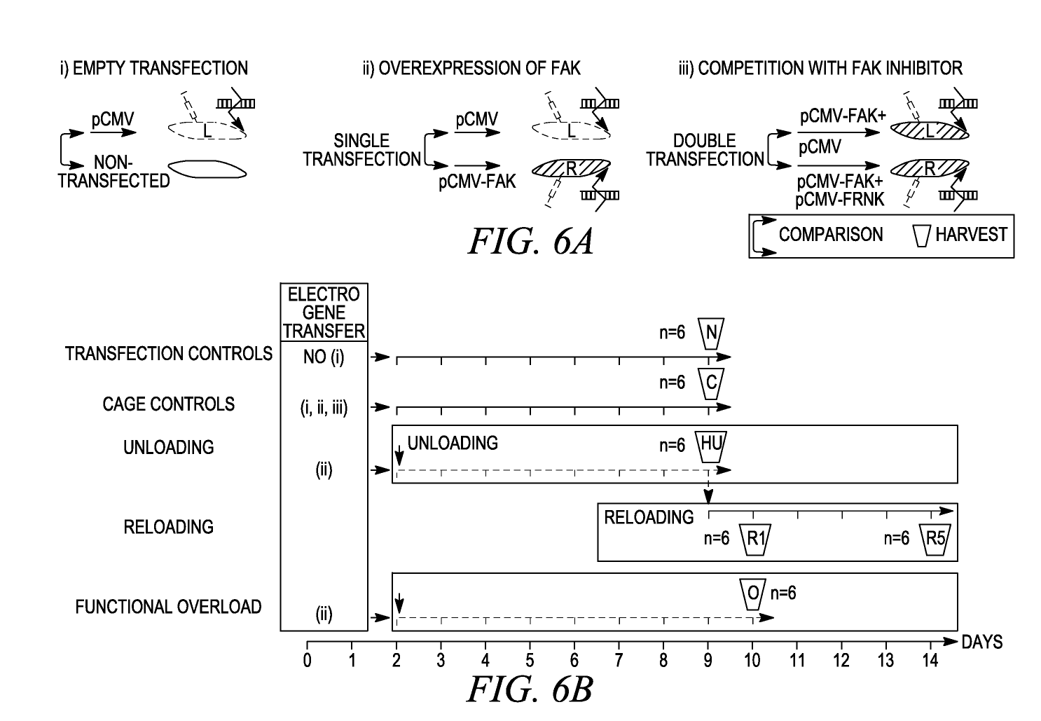

Unloading of soleus muscles by hindlimb suspension was essentially performed as described (Fluck et al., 2005). The suspension for 7 days was started 2 days after transfection. For the reloading intervention the animals were allowed to return to normal cage activity for 1 or 5 days after the period of unloading (see FIG. 6). A particular emphasis is put on the duration of experiments, the harvesting and the relevant comparisons of empty (pCMV), FAK (pCMV-FAK) and FRNK (pCMV-FRNK) transfected soleus muscles. Abbreviations: N, non-transfected muscle; C, cage control; HU, 7 days of hindlimb unloading; R1, 1 day of reloading after unloading; R5, 5 days of reloading after unloading; 0, 8 days of functional overload; n, number of animals per treatment. At the end of the respective protocol, rats were weighed, anesthetised and m. solei of both hindlimbs were rapidly dissected and weighed. 4-mm thick slices from the transfected belly portion were isolated, perpendicularly oriented on a cork and frozen in melting-isopentane. Samples were stored in sealed tubes at −80° C. until the subsequent analysis was carried out. Tenotomy of gastrocnemius muscle was performed at the University of Pavia (Italy) 2 days after transfection under anesthesia. A scalpel incision was made to the achilles tendon of the anestetized animals and secured with stitches. Soleus muscles were harvested 8 days after tenotomy.

Experimental design—In all cases, a paired design was adopted to allow intra-animal comparisons (FIG. 6). For the FAK overexpression experiments, right and left m. solei were transfected with the same amount of pCMV-FAK and pCMV plasmid, i.e. 35 μg in 50 μl respectively. For the FRNK coexpression experiment, one soleus muscle was transfected with a mix of pCMV-FAK (25 μg) and pCMV-FRNK (45 μg) in a final volume of 70 μL. The same relative amounts of pCMV-FAK (25 μg)/pCMV (45 μg) plasmid were injected in the contralateral muscle. The impact of gene electrotransfer on soleus muscle was analyzed, by comparing centrally-nucleated fibres, cell infiltration, ectopic tenascin-C abundance and gene expression between empty plasmid-transfected (i.e. pCMV) muscles to nontransfected muscles from a previous study (Fluck et al., 2005). Soleus muscles from the cage control group were analysed 7-9 days after gene electrotransfer for FAK overexpression. Appropriate statistical tests were employed as specified in the respective analysis.

Transcript profiling—Total RNA was extracted from pooled cryosections of transfected soleus portions and subjected to microarray analysis with Atlas Rat 1.2 cDNA microarrays (ATLAS™ 1.2 array, BD CLONTECH Basel, Switzerland) as described (Fluck et al., 2005). Raw data of all measured 1185 transcripts of a filter were normalized to the total sum of transcript signals on the filter and subjected to statistical analysis for microarrays (SAM) for a two class paired design (Dapp et al., 2004). Significant regulation of transcript expression within a gene ontology (GO) was analyzed by estimating the enrichment of co-incidental level changes by using a binomial test normalized to the p-value of a hypothetical symmetric up- and down-regulation within a GO (Excel in MS-Office for Windows XP, Kildare, Ireland). The p-values were visualized via Cluster and Treeview (http://rana.lbl.gov/EisenSoftware.htm) and assembled with CorelDraw X3 (Corel Corporation) and Powerpoint (MS-Office for Windows XP, Kildare, Ireland). Data sets are deposited under series numbers at Genomnibus.

Protein quantification in homogenate—Muscle sample preparation from cryosections into RIPA buffer, protein detection and quantification in immunoblots, and quantitative immunohistochemical experiments were carried as described (Fluck et al., 1999; Fluck et al., 2002; Giraud et al., 2005). Myosin heavy chain content in single fibres and whole muscle was assessed by highly resolving SDSPAGE electrophoresis and silver staining. A Wilcoxon test was applied to test the effect of pCMV-FAK plasmid gene electrotransfer vs. paired pCMV-transfected control on protein expression in homogenate. A two-way ANOVA was applied to test the effect of loading (normal, unloading, 1 day reloading, 5 days reloading) and transfection of the contralateral muscles with either of the plasmid.

Morphometry-Fibre cross sectional area and percentage of fibres with sarcolemmal FAK concentration was determined from FAK-stained cross-sections (Fluck et al.) using a microscopic station (Leica DMRB, Vienna, Austria) running under Analysis 5.0 software (Olympus Soft Imaging Solutions GmbH, www.olympus-sis.com). Pictures were taken at 20-fold magnification to cover the entire cross-section of soleus muscle. The area of FAK-positive and negative-fibre in the targeted muscle portion was estimated with a circumference method. On average, 980 fibres were counted from different muscles for each comparison. The mean area of FAK transfected fibres per total cross section was also determined.

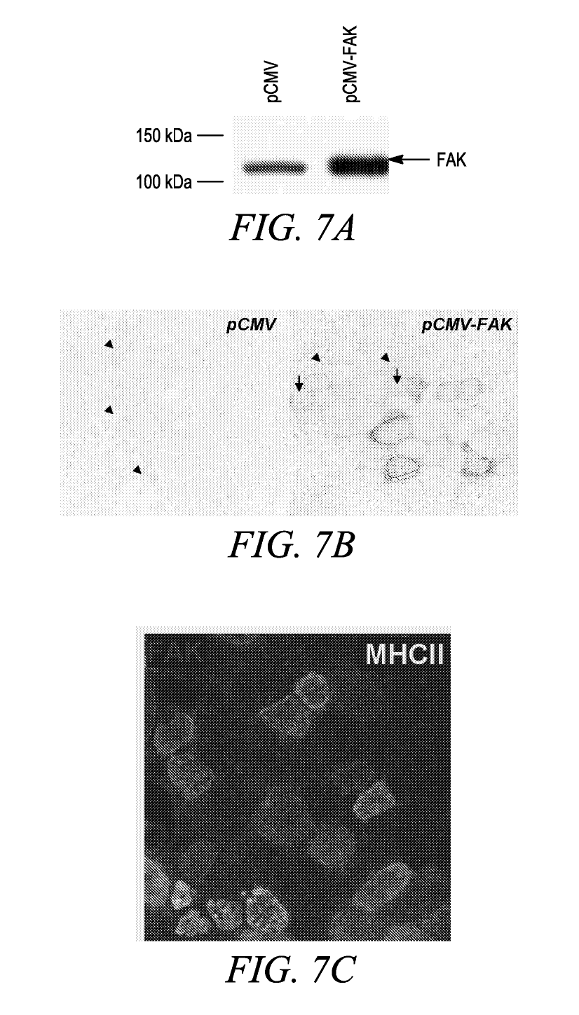

The percentage of fibres in FAK-transfected muscles with subsarcolemmal FAK localization was determined essentially as described (Fluck et al., 2002). In brief, microscopic pictures from the FAK stained cryosections were printed and fibres were classified into those with distinct sarcolemmal stain or showing an exclusive staining in the sarcoplasm (see FIG. 7). FAK protein in homogenate (A) and consecutive cross-sections (B) of empty-plasmid (‘empty’) and FAK-expression plasmid (‘FAK’) transfected contralateral muscle pairs of a rat. FAK protein in B is detected as an orange stain along with the counterstained nuclei. Arrowheads and arrows indicate cell infiltration and regenerating muscle fibres with central nuclei. Note the absence of FAK staining in empty-transfected fibres and the concentrated FAK staining at the sarcolemma and as punctuate stain in the sarcoplasm of FAK transfected fibres. C) Immunoflorescence double staining for FAK and type II MHC protein. The number of fibres falling into the 2 categories were counted and pooled between muscles with the same treatment. A Chi² test was applied to test differences in the frequency of the 2 fibre categories (MS-Office for Windows XP, Kildare, Ireland).

The mean cross-sectional area of fibres from the two categories was determined with a circumference method as described above after assignment of the fibres. Additionally FAK-staining and myosin heavy chain II double staining was visualized with immunofluorescence a Leica SP5 system using FAK and MHC II primary and Alexa fluor 488 and Alexa fluor 555-labelled secondary antibodies.

Myography-Contractile parameters in whole mounts and single fibres were assessed as described (D'Antona et al., 2003).

### Example 7

### FAK Overexpression, Modulation, Regulation and Relocalization

Muscle-fibre targeted FAK overexpression-Focal adhesion signalling in muscle fibres was manipulated via electrotransfer of a constitutively-active FAK expression plasmid (pCMV-FAK, FAK transfected) in the belly portion of the right soleus muscle. Adjustments were compared to transfections with empty plasmid pCMV (empty-transfected) in the left soleus muscle (FIG. 6). The ratio of FAK protein between FAK- and empty transfected soleus pairs was enhanced by 2.6-fold in the targeted muscle portion 8 days after gene electrotransfer (FIG. 7A). 23.4%±4.9% of total muscle cross section stained positive for FAK (FIG. 7B). No signal was detected in the empty-transfected left muscle (FIG. 7B). FAK overexpression was exclusively localized to muscle fibres and was associated both to the sarcolemma and the sarcoplasm (FIG. 7B/C).

Comparison to non-transfected rat soleus muscles (FIG. 6; Fluck et al., 2005) demonstrated that the gene electrotransfer procedure was associated with muscle regeneration. This was visualized by cell infiltration, the appearance of central nuclei and elevated hybrid fibre type I/II percentage (FIG. 7B/C). This resulted in sizeable transcript level changes which reproduced the adjustments seen with fibre damage (FIG. 8, Fluck et al., 2005). Graphical representation of the transcript level changes in the functionally-distinct gene family. Color code denotes the level of significance of coincidental level change of transcripts in the different load and gene transfer treatments as referred to in FIG. 6. This concerned a general upregulation of RNAs for protein turnover factors with a concomitant down-regulation of mitochondrial transcripts. The cell quantitative analysis of FAK-transfected muscle revealed that the fibre cross sectional area of FAK-positive fibres was 5% reduced over non-transfected fibres, i.e. 2,020.1±49.5 versus 2,123.4±16.7 μm².

Transcript profiling identified a general downregulation of RNAs in FAK overexpressing soleus muscle versus the empty-transfected contralateral control (FIG. 8). The majority of changes situated below 50%. A significant enrichment of mRNA down-regulation was evident for gene ontologies involved in oxidative metabolism, voltage-gated ion transporters, protein synthesis, proteolysis, the adhesion-cytoskeleton axes and G-proteins. Notable exception was the enhanced level of the message fore the slow-twitch specific SERCA2 calcium channel (Table 3).

TABLE 3
Soleus muscle mass after FAK overexpression and different treatments.
Summary of the detected transcript level changes with FAK
overexpression in the different loading treatments.

| | Plasmid | C | HU7 | R1 | R5 | 0 |
|---|---|---|---|---|---|---|
| Mass | CMV | 0.44 ± 0.04 | 0.30 ± 0.02 | 0.33 ± 0.02 | 02 0.47 ± 0.02 | 0.59 ± 0.05 |
| [mg/g] | CMV- | 0.50 ± 0.02 | 0.31 ± 0.03 | 0.37 ± 0.02 | 0.45 ± 0.02 | 0.52 ± 0.04 |
| | FAK | | | | | |
| number | | 6 | 6 | 6 | 6 | 7 |
| p value | | 0.06 | ns | <0.05 | ns | ns |

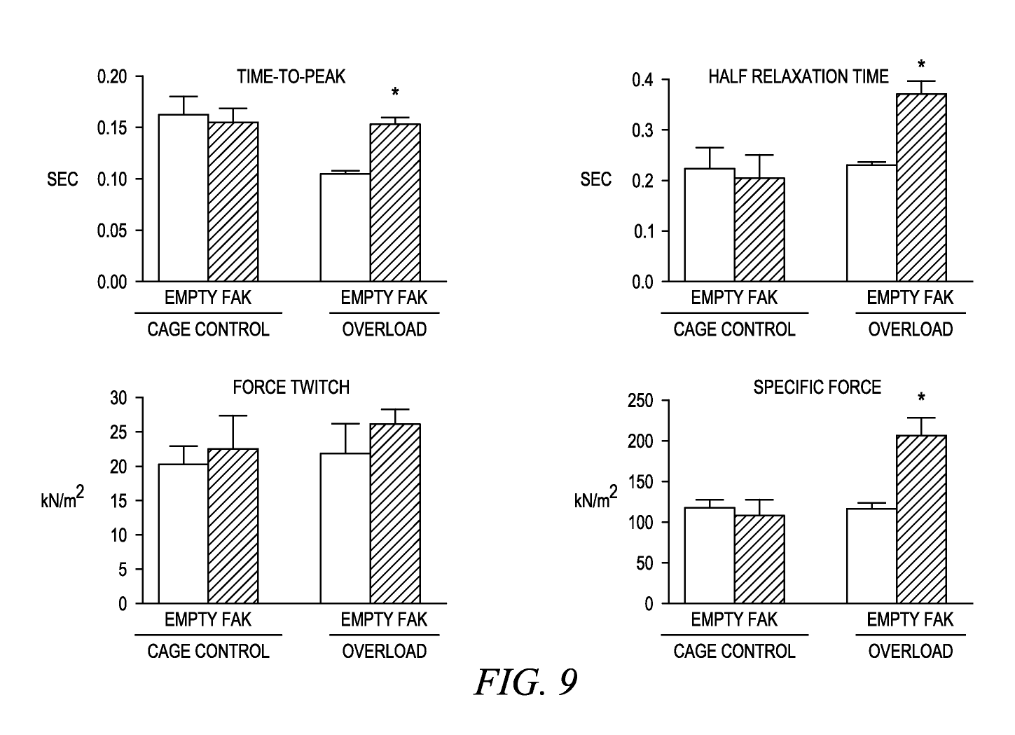

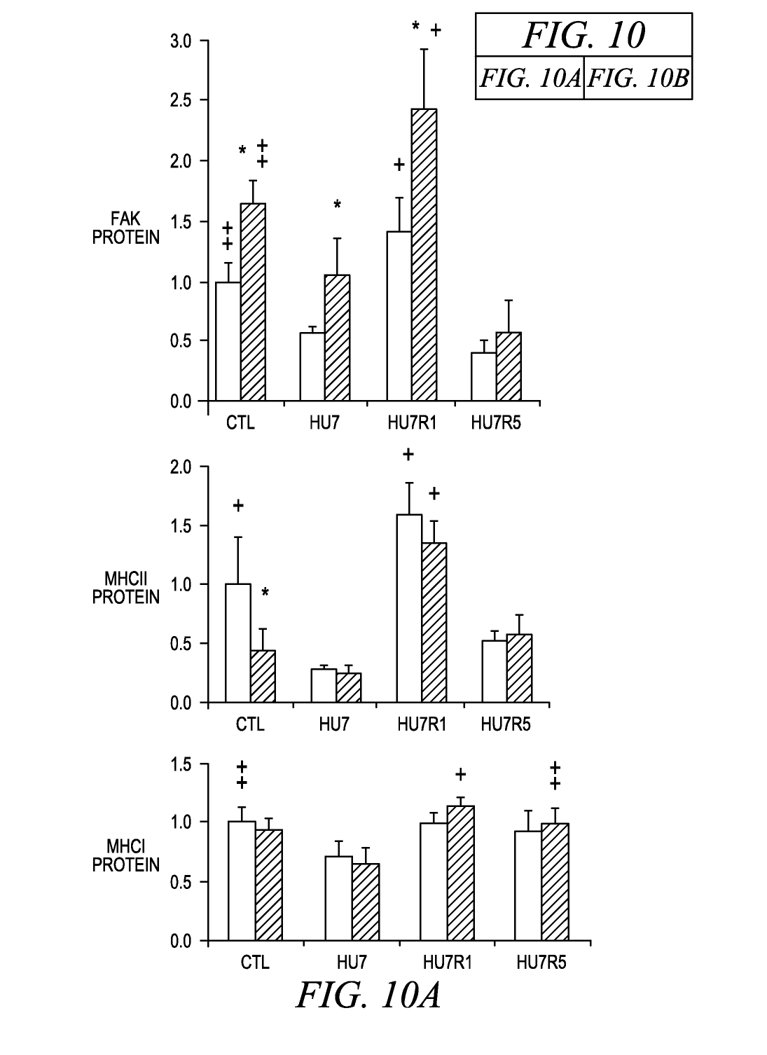

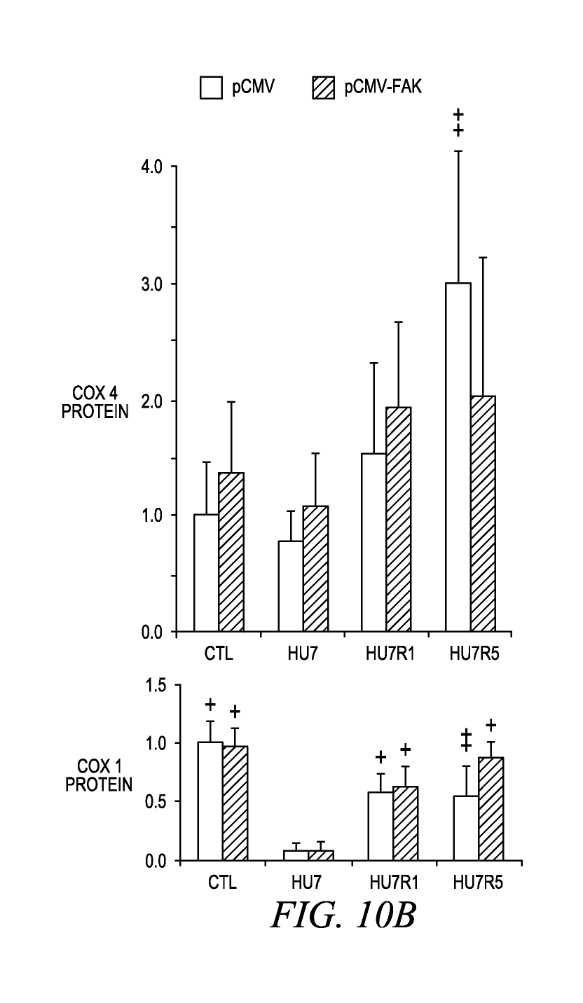

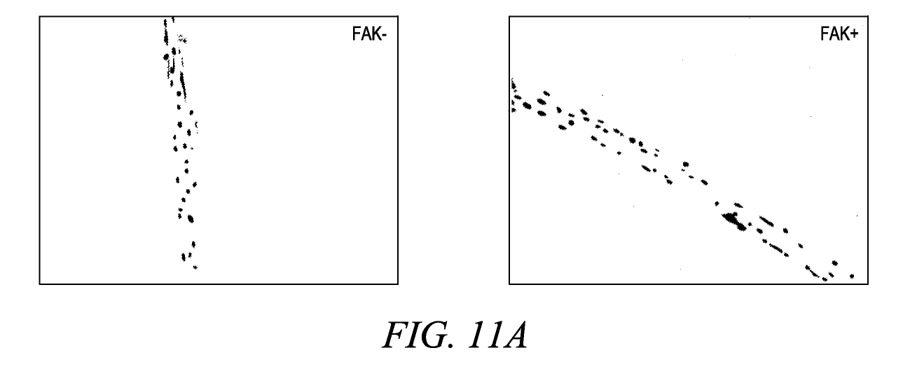

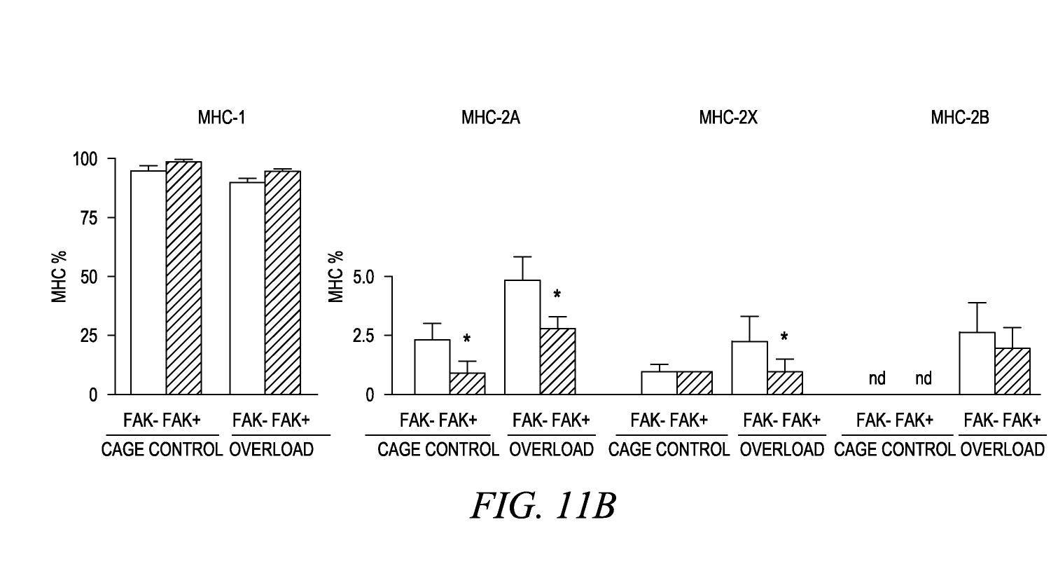

There was a trend for elevated soleus mass in FAK-overexpressing versus empty-transfected contralateral control muscle (table 3). Contractility was not significantly different between the FAK- and empty transfected soleus muscle (FIG. 9). A) Myograph measures witnessing slowed time-to-peak, and half relaxation time of single twitches and enhanced specific force of FAK-transfected muscle after functional overload but not in cage controls. Because transfection is confined to a portion of fibres only we assessed the effects of FAK overexpression at the single fibre level. These experiments did not bring about structural and functional effects of FAK-overexpression compared to intra-muscular and contralateral controls. The analysis did however indicate a 50%-reduction of the fast-type myosin heavy chain II A (MHCIIA) in transfected fibres with FAK overexpression. The content of the more abundant slow-type MHCl was unaltered (FIG. 10) [*, p<0.05; $, 0.05<p<0.10 vs. contralateral control (Wilcoxon-Test). +, p<0.05; ‡, 0.05<p<0.10 vs. same transfection after unloading (ANOVAFisher)]. These changes were confirmed by subsequent protein measures on whole muscle (FIG. 11). A) Microscopic picture of FAK-positive (FAK+) and FAK-negative (FAK−) fibre 8 days after FAK-transfection and functional overload. B) Mean of MHC isoform protein in single fibres of soleus muscle of cage controls and after functional overload. *, p<0.05 vs. contralateral control.

Modulation of FAK-dependent muscle control by un- and reloading—We tested whether muscle reloading after 7 days of unloading promotes the hypothesized FAK-dependent muscle expressional adjustment of gene expression. 1 and 5 days of reloading of empty-transfected soleus muscle reproduced the transcript and weight changes seen before with longer durations of suspension (Fluck et al., 2005; data not shown). Endogenous FAK protein levels in empty-transfected soleus muscles were reduced after 7 days of hindlimb unloading, and transiently increased after 1 day of reloading (FIG. 10). A similar behaviour was seen for MHCII and COX I protein.

With FAK-transfection, total FAK protein in right soleus muscle was elevated compared to the paired empty-transfected muscles after 1 day of reloading but returned to baseline levels after 5 days of the reloading stimulus (FIG. 10). FAK-dependent transcript expression in unloaded muscle reproduced the changes seen in cage controls (FIG. 8). Reloading of deconditioned muscle transiently enhanced the transcript levels for hypothesized FAK-dependent mRNAs related to mitochondria and oxidative metabolism, protein degradation and adhesion/cytoskeleton after 1 day. As well the mRNA levels of G proteins and the regain of muscle mass were transiently elevated with reloading and FAK overexpression (table 3). Immunoblotting experiments of key mitochondrial and sarcomeric factors revealed that neither of the elevated mRNAs for cytochrome c oxidase 1 and 4 (COX1, COX 4) translated into corresponding alterations of the encoded protein (FIG. 10). Nor was there a FAK dependent difference in MHC protein expression. The experimental setup would not allow myograph measures on site.

FAK regulation by chronic overload—We tested in subsequent experiments whether functional overloading of soleus muscle by tenotomy of the synergistic gastrocnemius muscle would promote a translation of the FAK-induced adjustments after (re)loading to a functional level. Based on the duration of the previous experiments we reasoned that 8 days of functional overloading would be a sufficient stimulus and specifically focused on contractile adjustments as these can be quantitatively analysed at the single fibre level.

FAK protein overexpression was readily detected in the FAK-transfected right but not the empty transfected left soleus muscle (FIG. 11). Mass of soleus muscle was not different between the FAK transfected and empty-transfected muscles (table 3). Similarly cross sectional area did not distinguish FAK-transfected from non-transfected fibres in the FAK-overexpressing soleus muscle. The myograph measures witnesses a shift of FAK-transfected muscles versus a slow contraction type. This was indicated by a slowed time-to-peak and half relaxation time of single twitches in FAK- vs. empty-transfected muscles (FIG. 9A) and earlier tetanic fusion (data not shown). This related to lowered fast myosin expression in FAK-transfected muscle fibres (FIG. 11B). Specific force was enhanced in FAK-transfected m. solei but was not different between FAK-transfected and non-transfected fibres in FAK-transfected right muscles.

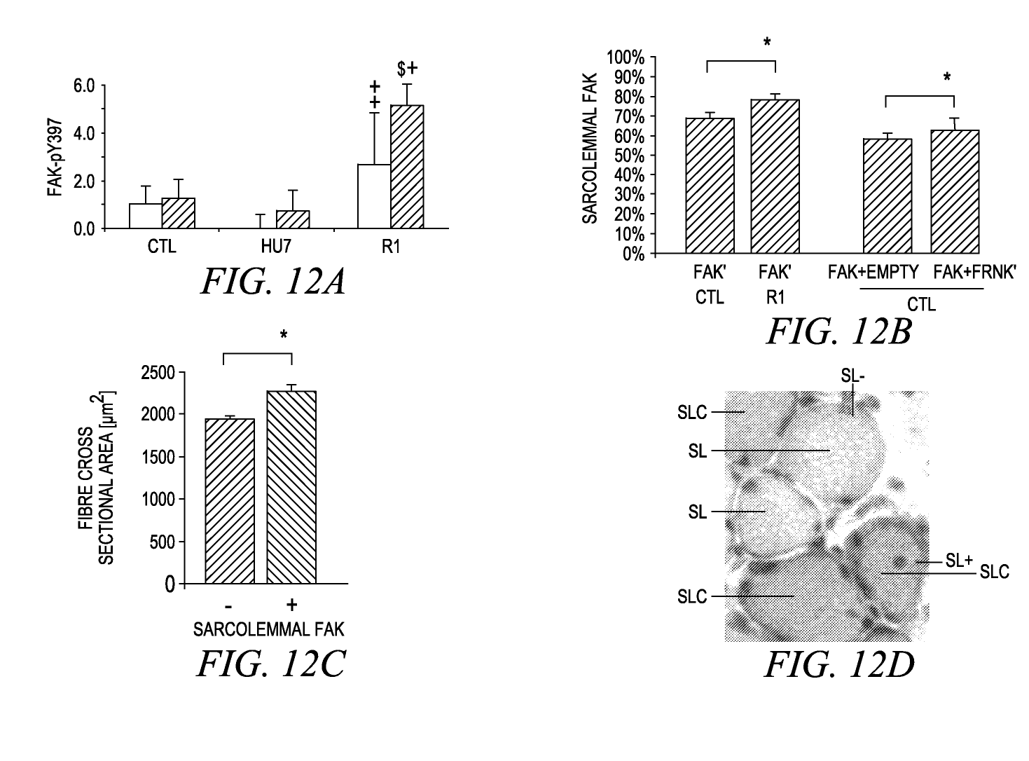

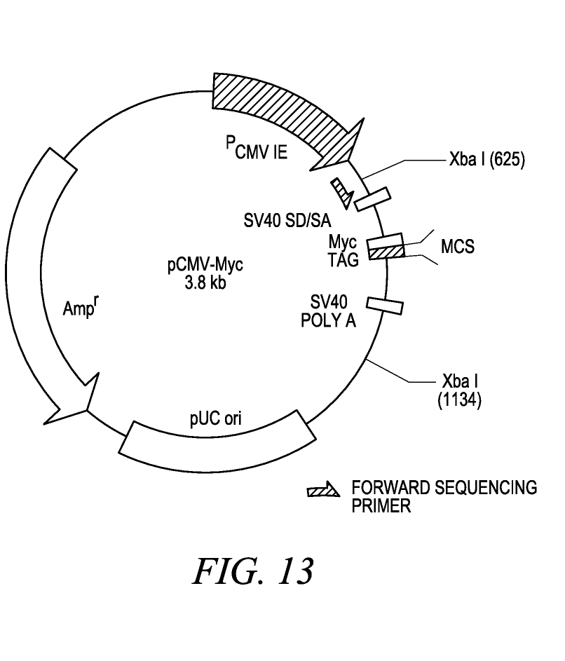

FAK-relocalization and load control of muscle—The contribution of FAK tyrosine phosphorylation at amino acid 397 (FAK-pY397) and re-localization to the sarcolemma to the observed effects in FAK transfected muscle was analysed. This testing was possible in cage controls and 1 day reloaded muscles where FAK overexpression was readily detectable. FAK-pY397 was low in muscles from cage control and unloaded muscle but was elevated 1 day after reloading in FAK-transfected versus contralateral muscles (FIG. 12A). A) FAK phosphorylation at tyrosine 397 with un- and reloading. B) Translocation of overexpressed FAK to the sarcolemma with one day of reloading and after cotransfection with FAK's autonomous competitor FRNK. C) Hypertrophy of fibres with strong sarcolemmal FAK staining after FRNK overexpression. One tailed repeated ANOVA: $: 0.05<p<0.10 vs contralateral control. +: p<0.05; ‡, 0.05<p<0.10 vs cage control. (Fisher HSD). D) Representative picture of FAK overexpressing fibres with (SL+) or lacking (SL−) sarcolemmal FAK protein. This corresponded to an elevated number of transfected fibres with subsarcolemmal FAK staining after 1 day of reloading versus cage controls (FIG. 12B).

Co-transfection experiments with FAK and its autonomous competitor FRNK were performed to interfere with FAK's action in soleus muscles of cage controls. The de novo expression of FRNK in muscle fibres reproduced the significant reinforcement of FAK staining at the sarcolemmal (FIG. 12B). Morphometric analysis showed that strong subsarcolemmal FAK localization coincided with 16%-increased mean fibre cross sectional area compared to muscle fibres with exclusive sarcoplasmic FAK expression (FIG. 12C). Co-incidentally, the transcript levels of FAK-regulated gene ontologies were enhanced from their reduced levels in the FAK-only transfected cage controls (FIG. 8).

### Example 8

### FAK is the Potential Coordinator of Contractile and Metabolic Differentiation in Slow-Oxidative Muscle Fibers Via a Load-Dependent Mechanism

Skeletal muscle's contractile and metabolic properties undergo pronounced differentiation upon the impact of recruitment-related stimuli. This gives rise to a spectrum of muscle phenotypes with differences in force production and fatigue resistance. The functional implication of load-regulated molecular pathways in such muscle conditioning is poorly understood (Fluck and Hoppeler, 2003). Towards this end our muscle-targeted transgenic investigation focussed on focal adhesion kinase since this molecule is one of a few signal transducers which post-translational modification and expressional regulation complies with a regulatory role in mechano-transduction in striated muscle (Durieux et al., 2007; Fluck et al., 1999; Fluck et al., 2002; Gordon et al., 2001; Quach and Rando, 2006). Previous investigations on focal adhesion kinase signalling did not allow revealing information on the normal physiology of FAK's molecular function since FAK gene ablation in the germline is of lethal consequence. Our somatic transgenic approach circumvents this limitation and provides combined molecular, cellular and functional evidence for load-dependent myocellular adjustments after FAK overexpression. This integrative analysis is thus first to single out the in vivo contribution of focal adhesion signalling to the gene-mediated regulation of load-dependent muscle characteristics.

The comparison to non-transfected soleus muscle identified gene transfer-induced myocellular degeneration-regeneration as the major co-variable of our approach (Durieux et al., 2004; Durieux et al., 2002; Rizzuto et al., 1999). These adverse effects on muscle were presented by the appearance of central nuclei and a sizeable drop in specific force of transfected m. solei muscles from 200 to 110 kN/mm2. The observed transcript signature with empty transfection reproduced the myocellular reprogramming seen with reloading damage of deconditioned soleus muscle (Fluck et al., 2005). These findings imply that the muscular adjustments to FAK overexpression have to be seen in the context of the enhanced plasticity due fibre repair. The inherent bias of somatic gene transfer was controlled by paired intra-animal comparisons allowing ‘to subtract’ the transfection interference. Limitations were also indicated concerning the transient nature of somatic FAK overexpression from the constitutive CMV-promoter after extended durations of reloading (Brooks et al., 2004). We handled this biological constraint by switching to the tenotomy model which allowed higher increments in loading early on after transfection. This layout and the integrative combination of muscle tests developed a high biological resolution power which allowed assigning distinct regulatory adjustments in muscle fibres as downstream to FAK function.

These experiments have identified the FAK-dependent regulation of distinct gene ontologies in soleus muscle. The reloading-induced transcript levels of the two gene families ‘mitochondrial oxidative metabolism’ and the ‘ECM-cytoskeletal axes’ are of particular interest. Both are associated with the metabolic and mechanical differentiation of frequently recruited muscle fibres (reviewed in Bozyczko et al., 1989; Fluck et al., 2002; Hoppeler and Fluck, 2003) and FAK-suppressed the corresponding transcript levels in cage controls and during unloading (FIG. 8). The notion of FAK's functional implication in mechano-regulation was best illustrated after functional overloading of m. soleus when FAK remained constitutively overexpressed (FIG. 11A). In this situation, the FAK-provoked expression changes of key elements of excitation-contraction coupling in cage controls, i.e. MHC II and SERCA2, translated to the functional level (FIG. 11). This load-dependent shift of FAK-transfected soleus muscle to a slower contraction speed with higher specific force (FIG. 9) could be assigned to the transfected fibre population by means of lowered MHC II isoform expression (FIG. 11). The drop in expression of all fast MHC isoforms with virtually unchanged expression of the major slow type MHC isoform in FAK-transfected soleus fibres highlights the suspected implication of FAK in the contractile differentiation of slow-fatigue fibres muscle. Collectively the data support that FAK exerts control over the character of frequently recruited soleus muscle fibres via load-dependent expressional regulation of energy fuelling and mechanical support of slow fibre contractions.

The absence of the FAK-promoted reduction of MHC type II protein in soleus muscle after unloading and reloading was a surprising given that unloading is expected to increase MHC II protein expression (Fluck et al., 2005). This finding contrasted the transiently elevated MHCII content with one day of reloading in both FAK- and empty-transfected soleus muscle and the elevated type II fibre content in transfected muscle. MHC II isoform expression in slow-contracting soleus muscle arise form muscle degeneration due to denervation or damage (reviewed in Fluck and Hoppeler, 2003). The former possibility can be excluded. These observations point to the modulation of damage in transfected muscle fibres as the obvious explanation of the observed alteration in sarcomere remodelling with FAK overexpression. This specifically implies that FAK promotes the faster reestablishment of the normal slow contractile characteristics of muscle fibres in this anti-gravitational muscle.

The probing of FAK's function in vivo with a non-constitutive FAK molecule also identified the regulation of a number of mRNAs for voltage-gated ion channels and G-proteins. These data witness that FAK is a broadly effective facilitator of the phenotypic control of anti-gravitational muscle via control over a spectra of biological processes (Campbell et al., 2001). Meanwhile the absence of a significant translation of elevated levels of the major mitochondrial subunits COX1 and 4 in FAK transfected muscle after reloading indicates that other factors which are missing in our somatic transgenic approach come into play to produce net mitochondrial biogenesis.

The load-dependent upregulation of transcript levels in FAK-transfected m. solei coincided with the augmented tyrosine phosphorylation of FAK at its main regulatory site of kinase activation and the translocation of FAK from the sarcoplasma to the sarcolemma (FIG. 12A/B). Interestingly, the coexpression of FAK with its autonomous competitor, FRNK, caused a similar relief from the ‘suppressive’ effect of FAK on transcript levels for ‘mitochondrial oxidative metabolism’ in cage controls, and reduced FAK's sarcoplasmic retention. Notably, the corresponding fibre population with elevated subsarcolemmal FAK content demonstrated hypertrophy (FIG. 12). These observations support the functional relevance of the previously reported induction of FAK phosphorylation by muscle loading (Fluck et al., 1999; Gordon et al., 2001) and the association of subsarcolemmal FAK protein levels with frequently recruited muscle fibres (Fluck et al., 2002).

The findings point out a novel mechanism involving a differential role of sarcoplasmic and subsarcolemmal FAK pools for the physiological control of metabolic and contractile features in slow tonic soleus muscle. This type of muscle shows a high degree of oxidative metabolic differentiation due to its frequent involvement in anti-gravitational support functions (Fluck et al., 2005). The results support that elevated sarcoplasmic FAK levels in absence of FAK activation exert a negative gene regulatory influence over metabolic functions of slow type contractions (FIG. 8). This can be reversed by muscle loading via tyrosine 397-phosphorylation and translocation to the subsarcolemma.

Microscopically examination with N-terminal FAK antibodies visualized the presence of FAK at the surface of myosin heavy chain bundles (FIG. 7B). This is compatible with the reported association of FAK to myofibrils of cardiac muscle cells (Fonseca et al., 2005). Interestingly, this interaction in cardiocytes appears preferential for FAK which is not phosphorylated at Tyr-397 and is reduced by mechanical stress when FAK relocates to costameres and Z-discs. The redistribution of FAK to the sarcolemma in hypertrophying fibres in our study upon co-overexpression of FAK with its C-terminal FRNK isoform suggest that the sarcoplasmic FAK pool is subject to a similar regulation in soleus muscle fibres. Collectively, the observations indicate an important spatial level of control for the physiological regulation of fibre type differentiation via focal adhesion signalling.

In summary, these experiments imply that controlled gene transfer combined with transcript profiling, morphometric and myographic analysis is suitable to pinpoint at the functional implication of single molecules in myocellular remodelling in vivo. The identification of novel downstream targets of focal adhesion signalling highlights that striated muscle plasticity is a powerful paradigm to elucidate physiologically important pathways. These results identify FAK as the long sought coordinator of contractile and metabolic differentiation in slow-oxidative muscle fibres via a new, load-dependent mechanism involving FAK's translocation from a myofibrillar compartment to the sarcolemma. Given the resemblance of FAK-associated events in skeletal, cardiac and smooth cells, our findings are of relevance for other contractile tissues as FAK may controls the mechano-dependent repair of the entire myogenic lineage (Mansour et al., 2004; Quach and Rando, 2006; Taylor et al., 2001).

### Example 9

### Representative Sequence

Provided hereafter is an example of a representative sequence. The underlined section is a tag.

(chicken FAK)

SEQ ID NO: 1

atggagcagaagctgatctccgaggaggacctgggatccatggcagcagc
ttaccttgatccaaacttgaatcatacaccaagttcaagtgcaaagacgc
acctcggtactgggatggagcgttccccgggggccatggagcgagtccta
aaggtttttcactactttgaaaacagcagcgagccaacgacgtgggccag
cattatccggcatggagatgctactgatgttcgaggcataatacagaaga
ttgtggactgtcacaaagtgaaaaatgtggcctgctatgggttgcgactc
agtcatctgcagtctgaggaggttcactggctgcacctggacatgggggt
atccaatgtgagagagaaatttgaactagcacatcctccagaagaatgga
aatatgaactgagaattcggtacctgcccaaaggatttctaaaccagttc
actgaggacaaaccaactttaaattttttctatcagcaggtgaaaaatga
ctatatgttagaaatagcagatcaagtggaccaggaaattgctttgaaac
taggttgccttgaaatcaggagatcctacggagagatgagaggcaatgca
ttagagaagaaatccaactatgaagtgctagaaaaagatgtcggtttaag
acgattttttccgaagagtttgctagattcagtgaaggccaaaacactac
gaaaattaatccaacagacatttcgacaatttgccaacctcaacagagaa
gaaagtattttgaaattctttgagatcctctctccagtgtacagatttga
caaggaatgcttcaagtgtgcccttggttcaagctggattatttcagtgg
agctggcaattggcccagaggaaggaatcagctaccttacagacaagggt
gcaaatccaactcacctggcagattttaatcaagtacaaactattcagta
ttcaaacagtgaagacaaggacagaaaagggatgttgcaactgaagatag
ctggtgcacctgagcctctgacagtgacagcaccatccttaaccattgca
gagaatatggctgacttgatagacggatactgccgactggtgaatggagc
cacgcaatcttttattatcaggccacagaaagaaggtgaaagagctttac
catcaataccaaagctggccaacaatgagaagcaaggagtaaggtcgcac
acagtctctgtatcagaaacagatgactatgcagagataatagatgaaga
agatacttatacaatgccatcaaccagagattatgaaattcaaagggaga
gaattgaactggggcgctgcattggtgaaggacagtttggagatgtgcac
caaggaatttacatgagtccggaaaatccagctatggctgtagcaatcaa
aacatgtaaaaactgcacctcagacagcgttagagaaaagttcctacaag
aagccttaacaatgcgtcagtttgatcatcctcacattgtgaagctcatt
ggagttattacagaaaacccagtgtggataatcatggagctctgtacact
tggagagttgagatcgtttctgcaagtaagaaaattcagcttggacctgg
cctccctcatcctctacgcttaccagcttagcacagcacttgcttaccta
gagagcaaaagatttgtacatagagatattgctgctaggaacgtgctggt
atctgccactgactgtgtgaaattgggtgactttggcttatcccgataca
tggaagacagtacttactataaagcttccaaaggaaagttacctatcaaa
tggatggctccagagtcaatcaacttccgacggtttacctcagcaagcga
tgtgtggatgtttggtgtgtgtatgtgggagatcctgatgcatggggtaa
agcccttccagggagtgaaaaataatgatgttattggtcggattgagaac
ggtgagcggctccccatgcctccgaactgccctcccaccctctacagcct
tatgaccaagtgctgggcatacgaccctagtagacgacccaggtttactg
aacttaaagcacaactcagtacaatactggaggaggagaagctgcagcaa
gaggaacgaatgagaatggaatccaggcgacaagtcacagtatcctggga
ctcaggaggatcagatgaagctcctcccaagcccagcaggcctggttacc
ccagcccaaggtccagtgaagggttttatccgagtcctcagcatatggta
cagccaaatcactaccaggtatctggctactctggttctcatgggatacc
agccatggcaggcagcatttatcctgggcaagcttctctcttggatcaaa
cagattcctggaaccatcgacctcaggaagtatcagcatggcagccaaac
atggaggattcgggcactttggatgtacgaggaatggggcaggttctgcc
cacacatctcatggaggagaggttaataagacaacagcaagagatggaag
aagatcaacgctggcttgagaaagaggaacgattcctggtaatgaaacct
gatgtgcggctctccagaggcagcattgaacgggaggacggaggtctcca
gggcccagctggtaaccagcacatatatcagcctgtgggtaaaccagatc
atgccgctccaccaaagaagccccctcgccctggagccccccacttgggc
agcctcgcgagcctgaacagccccgtggacagctacaacgaaggcgtgaa
gatcaagccacaggaaatcagccctcctcctacggccaacctggaccgct
ccaatgacaaagtctatgagaatgtaaccgggctggtgaaagctgtcata
gagatgtccagtaaaatacagccagctccgccagaggagtacgtgcccat
ggtaaaggaggttggcttggcgctgagaaccttgctagcaacagtggatg
agtcgctgccagtgcttcctgcaagcacccacagagagattgagatggcc
cagaaactgctgaactctgacctggctgagctcattaacaagatgaagct
ggcccagcagtacgtcatgaccagcctgcagcaggagtacaagaagcaaa
tgctgacggctgctcacgctctggctgtggatgccaagaacttgctggat
gtcatcgatcaagccagactgaaaatgatcagccagtccaggccccac
taa

### Example 10

Provided hereafter is an example of a representative protein sequence. The underlined section is a tag.

Met E Q K L I S E E D L G S Met A A A Y L D P N L
N H T P S S S A K T H L G T G Met E R S P G A Met
E R V L K V F H Y F E N S S E P T T W A S I I R H
G D A T D V R G I I Q K I V D C H K V K N V A C Y
G L R L S H L Q S E E V H W L H L D Met G V S N V
R E K F E L A H P P E E W K Y E L R I R Y L P K G
F L N Q F T E D K P T L N F F Y Q Q V K N D Y Met
L E I A D Q V D Q E I A L K L G C L E I R R S Y G
E Met R G N A L E K K S N Y E V L E K D V G L R R
F F P K S L L D S V K A K T L R K L I Q Q T F R Q
F A N L N R E E S I L K F F E I L S P V Y R F D K
E C F K C A L G S S W I I S V E L A I G P E E G I
S Y L T D K G A N P T H L A D F N Q V Q T I Q Y S
N S E D K D R K G Met L Q L K I A G A P E P L T V
T A P S L T I A E N Met A D L I D G Y C R L V N G
A T Q S F I I R P Q K E G E R A L P S I P K L A N
N E K Q G V R S H T V S V S E T D D Y A E I I D E
E D T Y T Met P S T R D Y E I Q R E R I E L G R C
I G E G Q F G D V H Q G I Y Met S P E N P A Met A
V A I K T C K N C T S D S V R E K F L Q E A L T
Met R Q F D H P H I V K L I G V I T E N P V W I I
Met E L C T L G E L R S F L Q V R K F S L D L A S
L I L Y A Y Q L S T A L A Y L E S K R F V H R D I
A A R N V L V S A T D C V K L G D F G L S R Y Met
E D S T Y Y K A S K G K L P I K W Met A P E S I N
F R R F T S A S D V W Met F G V C Met W E I L Met
H G V K P F Q G V K N N D V I G R I E N G E R L P
Met P P N C P P T L Y S L Met T K C W A Y D P S R
R P R F T E L K A Q L S T I L E E E K L Q Q E E R
Met R Met E S R R Q V T V S W D S G G S D E A P P
K P S R P G Y P S P R S S E G F Y P S P Q H Met V
Q P N H Y Q V S G Y S G S H G I P A Met A G S I Y
P G Q A S L L D Q T D S W N H R P Q E V S A W Q P
N Met E D S G T L D V R G Met G Q V L P T H L Met
E E R L I R Q Q Q E Met E E D Q R W L E K E E R F
L V Met K P D V R L S R G S I E R E D G G L Q G P
A G N Q H I Y Q P V G K P D H A A P P K K P P R P
G A P H L G S L A S L N S P V D S Y N E G V K I K
P Q E I S P P P T A N L D R S N D K V Y E N V T G
L V K A V I E Met S S K I Q P A P P E E Y V P Met
V K E V G L A L R T L L A T V D E S L P V L P A S
T H R E I E Met A Q K L L N S D L A E L I N K Met
K L A Q Q Y V Met T S L Q Q E Y K K Q Met L T A A
H A L A V D A K N L L D V I D Q A R L K Met I S Q
S R P H Stop

### Example 11

### Methods and Materials for Examples 11-13

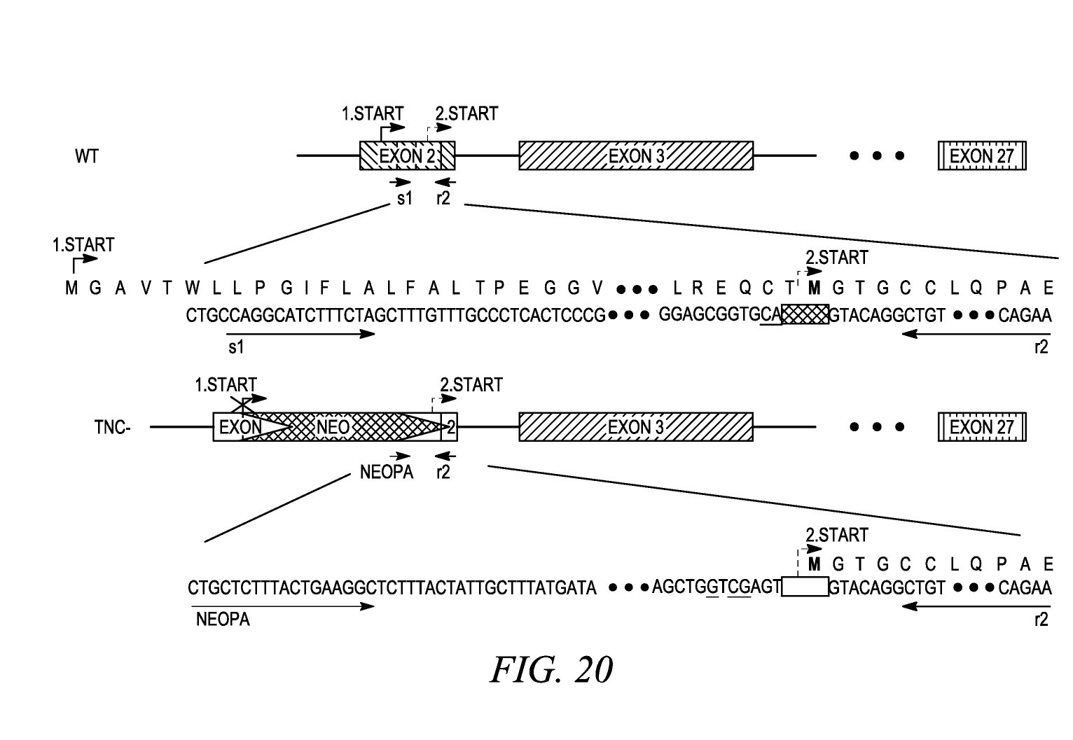

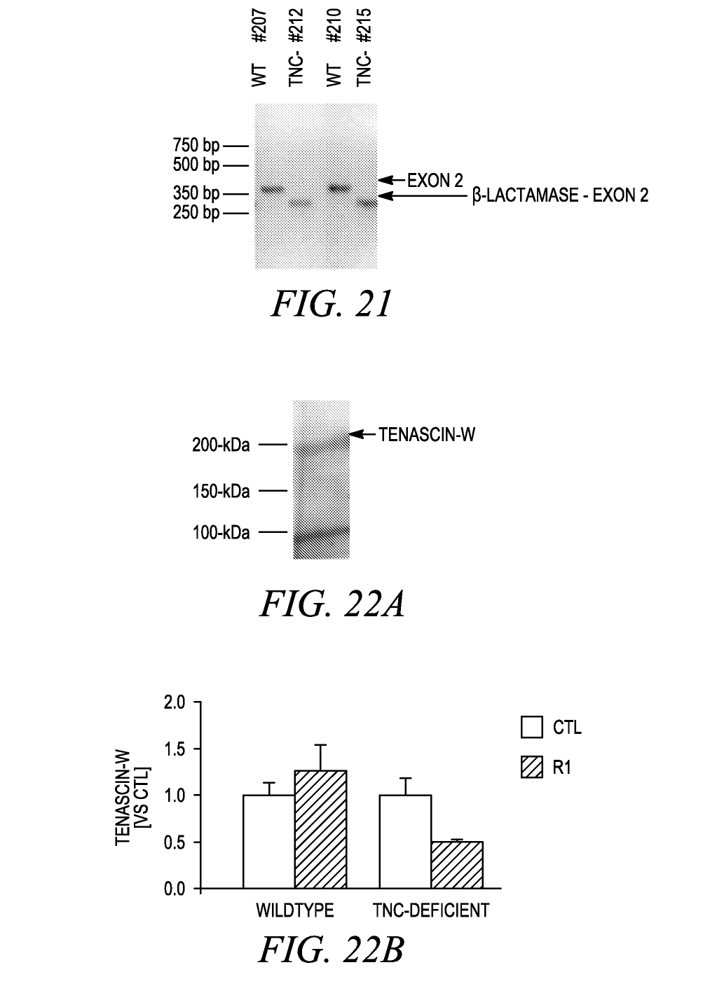

Animals—Male TNC-deficient mice of the 129/SV strain with the targeted insertion of a β-lactamase cassette in the NcoI site of exon 2 of the TNC gene were used for the study. Animals were derived from the original strain and back-crossed with wildtype 129/SV mice (Institut für Labortierkunde, University of Zurich). Genotype was determined with PCR on tail DNA as described. For details see FIG. 20. FIG. 20 shows Genotyping of ‘TNC-deficient’ mice. A) Construction of the mutated TNC translation initiation site. Sketches depicts the organization of the TNC gene on chromosome 4 (Genbank X56304) in wildytpe (WT) and TNC deficient mice (TNC-) with a particular focus on the targeted exon 2. Below each sketch, the nucleic acid and amino acid sequence (larger font) is given. In wildtype mice, exon 2 bears the initial start codon (1. start) which is followed by the signal peptide (italics). In TNC-deficient mice, exon 2 was modified by the insertion of a beta-lactamase gene cassette in the proximal and distal of three NcoI sites. This is indicated by lines in the exon 2 box. The beta-lactamase sequence in exon 2 of the TNC-deficient mouse (neo) is given in grey. The localization of the three primers (s1, r2 and neopa) used for PCR genotyping and sequencing are given by arrows in the sketch and shadowed font in the nucleotide sequence. Consensus sequences for translation initiation are underlined. The codon for the start methionin is given in bold. Note that there is an ‘in frame’ consensus translation initiation (2. start) in wildtype and TNC-mice which is 141 amino acids downstream of the original start. B) PCR-based genotyping. PCR was performed on isolated tail DNA (DNAeasy Tissue kit, Qiagen). The amplicons serving for the identification of wildytpe (435 bp) and TNC-mice (340 bp) are indicated. The PCR amplicons were also verified by cloning of into TOPO-PcrII vector (BD Biosciences Clontech) and DNA sequencing (Microsynth, Balgach, Switzerland).

Bio-reagents—Bacterial clones carrying plasmid for the CMV-driven expression of the 190-kDa chicken TNC isoform, pcDNAI-chTNC, or empty vector (pcDNAI) were received from Ruth Chiquet-Ehrismann (FMI, Basel, Switzerland). Slab cultures were shipped to Plasmidfactory (Bielefeld, Germany, www.plasmidfactory.de) for propagation and isolation of endogen-free plasmid. The identity of plasmids was verified by sequencing of the insert (Microsynth, Balgach, Switzerland). Beta-galactosidase plasmid was prepared as described in Durieux, A. C., Bonnefoy, R., Manissolle, C. & Freyssenet, D. (2002) Biochem Biophys Res Commun 296, 443-450.

Monoclonal TNC antibodies from rat hybridomas (mTN12), mouse hybridomas (Tn20) and Tenascin-W antibody were received from Matthias Chiquet and Ruth Chiquet-Ehrismann (FMI, Basel, Switzerland). Polyclonal antibody C24230 (BD Transduction Laboratories, Basel, Switzerland) was used to detect cyclin A isoforms. Polyclonal MyoD antibody (sc-304) was from Santa Cruz Biotechnology. A panel of horse radish peroxidase-coupled secondary antibodies (i.e. HRP-conjugated), was used to detect the immunoreactivity of the former antibodies. This concerned A-5595 for the rat IgG (Sigma-Aldrich, St. Louis, Mo., USA), A-2304 for the mouse IgG (Sigma-Aldrich, St. Louis, Mo., USA) and 55676 for rabbit IgG (ICN Biomedicals Inc., Aurora, Ohio, USA). The specificity of protein detection was tested against extracts with high expression of the protein of interest and with incubations omitting the primary antibody.

Cage controls and reloading of soleus muscle—Animals were acclimatized to housing in single cages before the experiments. After one week, animals were assigned to the reloading group (designated ‘R1’) or the cage control group (‘CTL’). De-conditioning of hindlimb muscles for 7 days by unloading, reloading and harvesting of muscle pairs was carried out as described. Unloading causes a reduction in whole body mass and soleus muscle mass. These parameters did not return to baseline with one day of reloading. In order to control for general effects on body mass, muscle mass was normalized to body mass. All procedures were approved by the Animal Protection Commission of the Kanton Bern, Switzerland and were carried out according to the latest guiding principles for research.

Muscle-targeted TNC Knock-In—Mice were anesthetized with 1-5% isofluran. The central portion of tibialis anterior muscle was injected with 30 μg of plasmid in 30 μl of 0.9% NaCl. After a 5-minute incubation period electropulses (3 trains of 100 pulses of 100 sec each at 50 mA) were delivered with needle electrodes using a GET42 electropulser (E.I.P. Electronique Informatique du Pilat, 42660 Jonzieux, France, www.elecinfopilat.com) as described in Durieux, A. C., Bonnefoy, R., Manissolle, C. & Freyssenet, D. (2002) Biochem Biophys Res Commun 296, 443-450. The overexpression experiments with CMV driven vector were carried out in a paired design; empty pcDNAI plasmid was injected in the left tibialis anterior muscle and corresponding amounts of pcDNAI-chTNC were deposited in the contralateral right muscle. Muscles were harvested 1, 2, 4 and 7 days after the transfection, trimmed to the transfected part and frozen for subsequent analysis as described.

In control experiments, electro-pulsing was performed after injection of betagalactosidase plasmid. Tibialis anterior, extensor digitorum longus, gastrocnemius and soleus muscles were harvested 7 days after the intervention and frozen. The occurrence of muscle damage was verified by the appearance of cell infiltration and central myonuclei in hematoxylin-stained muscle cross sections.

Muscle fiber structure—Fiber type composition and mean cross-sectional area of slow and fast-type muscle fibers were determined with standard morphometry on cross-sections from the muscle belly portion after immunostaining for fast- and slow-type muscle fibers. Slides were visualized at a 40-fold magnification on a microscopic station (Leica DMRB, Vienna, Austria) and pictures of visual fields were taken in a random initiated, systematic manner with a digital camera running under Analysis 5.0 software (Olympus Soft Imaging Solutions GmbH, www.olympus-sis.com). The number of fast and slow-type muscle fibers per square of 150×150 micrometer was determined using the forbidden line rule. The area of fibers of each type was determined by point counting using a 15×15 micrometer mesh. On average, 224 muscle fibers were counted per section. Cross-sectional area and the percentage of fast and slow fibers in each muscle cross-section were calculated with the Step-One program running on Windows 3.1 (MSOffice). The output values from the individual muscle sections were used to determine the mean values for fiber-type specific cross-sectional areas and percentage distribution of fiber types.

In situ testing of muscle contractility—Assessment of the contractile characteristics of the soleus muscles was carried out with modifications from Andrade et al (2004) using a muscle tester (SI systems Heidelberg, Germany) operated by a Powerlab system (ADinstruments, www.adinstruments.com). In brief, mice were anesthetized with subcutaneous injection of pentobarbital (50 mg/kg) and m. solei carefully removed. Muscles were equilibrated in gassed Tyrod-solution (121 mM NaCl, 5 mM KCl, 1.8 mM CaCl2, 0.5 mM MgCl2, 0.4 mM NaH2PO4, 24 mM NaHCO3, 0.5 mM glucose, 5% CO2, 95% O2) at 25° C. and transferred to the incubation chamber. Intact muscles were attached via clamps on one end to a micrometer screw and to a KG7-force transducer at the other. Muscle contractions were evoked by stimulation at 10V using an Ion Optix Myopacer (IonOptix Corporation, Milton, Mass. 02186, USA). Force signals were recorded by Chart 5 software (v5.4.1, ADinstruments, www.adinstruments.com). Muscle length was adjusted for optimal magnitude of single twitches. Single twitch and maximal tetanic force were measured after stimulation at 1 Hz for 0.4 milliseconds and 60 Hz for 4 seconds, respectively. Fatigue was determined with trains of tetanic contractions for 4 seconds interspersed with 4 seconds of rest. The time was recorded for force to drop below 50% of original tetanic force. Before each measure the gassed Tyrode solution was replaced and 2-5 minutes of rest was permitted. After the contraction protocol, muscles were frozen in N2-cooled isopentane and tibia length was determined with a millimeter ruler. Time-to-peak and duration time of the twitch contraction, half relaxation times of the tetani and maximal force for single twitches and fatigue values were extracted from the data set with customized software macros.

Transcript profiling—Microarray experiments were carried out with a validated, custom designed ATLAS™ cDNA nylon filter on total RNA as described. Each of the filters held cDNA probes for 222 mRNAs covering selected cDNA probes for gene ontologies involved in major muscle functions and TNC-associated-pathways. The curation of transcripts to a gene ontology (GO) was based upon the information available for the associated biological process through the electronic literature (http://www.expasy.org/sprot/ and http://www.ncbi.nlm.nih.gov/sites/entrez). Data sets and platform design were deposited in compliance with the minimal information about a microarray experiment (MIAME) under accession codes GSE8551, GSE8549, GSE8550 and GSE8552 at Gene Expression Omnibus (GEO; http://www.ncbi.nlm.nih.gov/geo). Statistical analysis of expression data was carried out using ‘statistical analysis for microarrays’ (SAM) and the subsequent assessment of global themes of coregulation: Differentially expressed RNAs between genotypes in cage controls were determined from RNA signals after normalization to the total filter signal. P-values were adjusted for false-discovery rate of 5%. Enrichment of a certain GO in the genotype effect on transcript expression was defined as a significant co-directional level change of relevant transcripts based on a sign-test. This was calculated with the ‘binomialdist’ function of MS-Excel (MS-Office for Windows XP, Kildare, Ireland).

Genotype differences in the ‘reloading response’ of transcript expression were evaluated using the ‘R1 vs CTL’ ratio of normalized transcript levels. This ratio was calculated for each genotype and reloading sample by relating the normalized transcript signals from the R1 experiment to the respective mean of transcript signals in cage controls (CTL). The ‘R1 vs CTL’ ratios of significantly altered transcripts were logarithmized, median-centered and subjected to hierarchical cluster analysis as described Fluck, M., Schmutz, S., Wittwer, M., Hoppeler, H. & Desplanches, D. (2005) Am J Physiol 289, R4-14. (http://rana.lbl.gov/index.htm?software/manuals/ClusterTreeView.pdf) and analyzed for co-regulation of transcript level adjustments within a GO (see above). Additionally, the genotype-dependent reloading response of transcript levels per GO category was compared to the situation in cage control. ‘Inversed’ genotype-regulation of the reloading response was declared when the ‘R1 vs CTL’ ratio was significantly different between the ‘TNC-deficient’ and ‘wildtype’ genotype (sign-test) and pointed in the opposite direction as seen in cage controls (‘CTL’).

Immunoblotting—Sample preparation, electrophoresis on a 7.5% SDS-Polyacrylamide gel, western blotting and quantitative immunodetection was carried out as described (7, 43) except that ultra-sensitive enhanced chemoluminescence was used (Pierce, Supersignal-Femto). The panel of primary and HRP-conjugated secondary antibodies being used is specified under the paragraph “bio-reagents”.

For quantitative analysis, 20 μg of protein, of samples to be compared were loaded on the same gel to reduce assay-to-assay variability. Signals were background-corrected for each gel, related to the mean of the respective controls and pooled between the different experiments. Statistical tests were performed as described in the figure legends.

Statistics—Statistical tests were carried out as described in the individual sections and legends. Individual data for this testing were assembled in MS-Excel (Kildare, Ireland). Probability-based statistical tests were carried out with Statistica (StatSoft, Inc. version 6, www.statsoft.com). ANOVA analysis and post-hoc testing was applied to determine genotype, age and treatment effects. Statistical significance was assumed at p<0.05; with 0.05≦p<0.10 being considered as a trend.

Image processing—Photographs of slides and gels were downloaded from the original software into CorelDraw version 10 and PowerPoint (MSOffice for Windows XP, Kildare, Ireland) for trimming and labeling.

### Example 12

### Results from Experiments

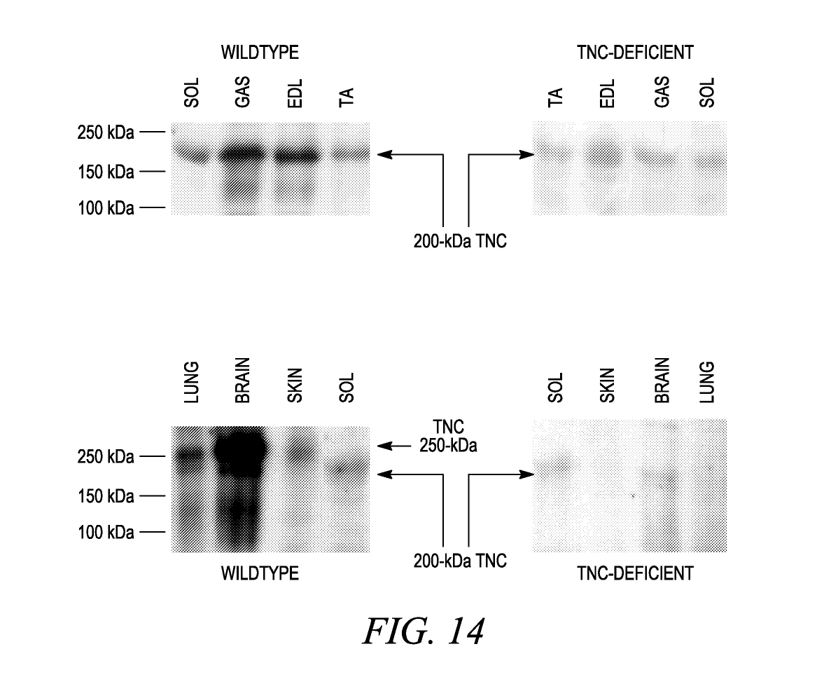

Tenascin-C isoform expression in wildtype and transgenic mice—Control experiments demonstrated that TNC isoform expression distinguished muscle from non-contractile tissues. In wildtype mice, leg muscles showed a variable expression of the small 200-kDa TNC isoform (FIG. 14A) while in lung, brain and skin the large 250-kDa TNC isoform was the predominant isoform (FIG. 14B). TNC expression was blunted in non-contractile tissues of transgenic littermates. However a 200-kDa TNC-immunoreactive band remained detectable at a 10-fold lower level in muscle tissue of TNC-deficient mice (FIG. 14B).

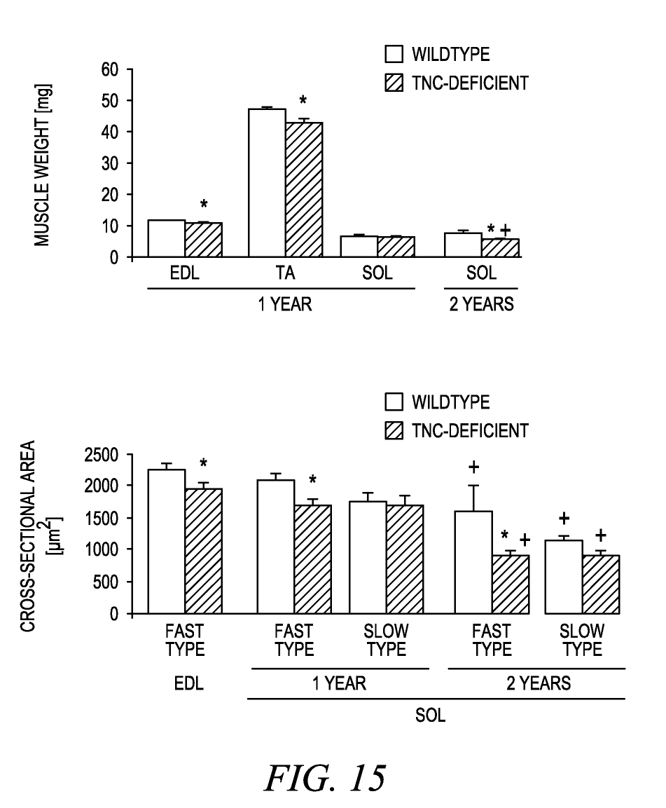

TNC-deficient mice demonstrate fast muscle fiber atrophy—One year-old TNC-deficient mice demonstrated reduced mass of the pure fast muscle type tibialis anterior and extensor digitorum longus (FIG. 15A). At this age no genotype difference was seen in the mixed slow/fast m. soleus. Quantitative microscopic analysis demonstrated a selective reduction of mean cross-sectional area for fast-type muscle fibers in extensor digitorum longus and soleus muscles of TNC-deficient mice (FIG. 15B). Contractile property measurements of m. soleus showed a significant slowing of muscle contractions in TNC deficient mice (table 4).

TABLE 4
Genotype effect on contractile properties of m. solei at one year of age

| Parameter | WT | TNC- |
|---|---|---|
| force single twitch [mN] | 0.8 ± 0.4 (6) | 0.9 ± 0.2 (6) |
| tetanic force [mN] | 11.4 ± 4.2 (3) | 9.2 ± 1.7 (6) |
| time-to-peak [msec] | 31.8 ± 3.7 (4) | 36.4 ± 1.9 (7)* |
| contraction duration [msec] | 24.7 ± 2.8 (5) | 29.2 ± 1.6 (7)* |
| half relaxation time [msec] | 20.3 ± 0.5 (4) | 31.8 ± 3.6 (6)* |
| fatigue [sec] | 57.3 ± 12.9 (3) | 52.0 ± 5.7 (5) |
| tibia length [mm] | 19.8 ± 0.6 (4) | 20.7 ± 0.3 (7)* |

Table 4 summarizes the mean and standard error of contractile parameters in soleus muscle of one-year-old wildtype (WT) and TNC-deficient mice. Numbers in brackets indicates the biological replicates per observation. *, significant difference between genotypes at p<0.01. ANOVA with HSD post-hoc test.

Further examination revealed that atrophy of fast soleus muscle fibers in TNC-deficient mice was progressive and became evident at the whole muscle level after two years of age (FIGS. 15A&B).

Transcript adjustments with TNC-deficiency—Experiments were performed to identify the contribution of expressional reprogramming of muscle-relevant factors to fast-fiber atrophy in soleus muscle. Transcript profiling identified a general up-regulation mRNA levels in m. solei of one-year old TNC-deficient cage controls (i.e. 60 of 63 affected mRNAs, p=4E-15). The major theme concerned the up-regulation of gene transcripts for gene ontologies (GOs) associated with the myofiber compartment, focal adhesion and angiogenesis (Table 5). This included several factors being associated with slow type fibers. At two-years of age, a majority of genotype differences in muscle mRNAs were preserved, except for those associated with myofibers.

TABLE 5
Gene ontologies with shifted expression
with TNC-deficiency in soleus muscle

| Gene ontology | gene counts | P |
|---|---|---|
| novel TNC targets | 45 | <0.001 |
| Adhesion | 12 | <0.001 |
| Angiogenesis | 14 | <0.001 |
| pro-angiogenic | 9 | <0.01 |
| proliferation | 12 | <0.001 |
| myofiber-associated | 23 | <0.001 |
| ECM-sarcomere axes | 5 | <0.01 |
| myogenesis | 9 | <0.01 |

Table 5 shows gene ontologies (GO) which transcripts showed unidirectional TNC-genotype differences in expression in soleus muscle of one-year old mice. All enriched GOs demonstrated enhanced expression levels of transcripts. For a comprehensive list of the affected transcripts see table 6.

TABLE 6
Shift in transcript expression with TNC-deficiency in mouse m. solei

| Gene | Genbank | TNC vs WT | GO1 | GO2 | GO3 |
|---|---|---|---|---|---|
| EphB3 | Z49086 | 1.03 | ECM | | |
| Ephrin B1 | U12983 | 1.05 | ECM | | |
| Integrin a8 | AF041409 | 1.39 | ECM | | |
| integrin b5 | AF043256 | 1.04 | ECM | | |
| laminin a3 | X84014 | 1.47 | ECM | | |
| laminin a4 | U59865 | 1.09 | ECM | | |
| laminin b2 | U42624 | 1.09 | ECM | | |
| laminin g2 | U43327 | 1.07 | ECM | | |
| MMP-2 | M84324 | 1.04 | ECM | | |
| Tenascin-X | X73959 | 1.11 | ECM | proliferation | |
| # integrin b1 | Y00769 | 1.05 | ECM | ECM-sarcomere | |
| laminin a2 | U12147 | 1.04 | ECM | ECM-sarcomere | |
| # Desmin | L22550 | 1.14 | myofiber | ECM-sarcomere | myogenesis |
| # MHC I | AY056464 | 1.09 | myofiber | ECM-sarcomere | |
| MHC IIX | AJ293626 | 1.15 | myofiber | ECM-sarcomere | |
| ALDO A | Y00516 | 1.01 | myofiber | energy metabolism | glycolysis |
| Enolase 2g | X52380 | 1.13 | myofiber | energy metabolism | glycolysis |
| Carbonic anhydrase 3 | M27796 | 1.03 | myofiber | energy metabolism | mitochondria |
| LDH 3 | M17587 | 1.16 | myofiber | energy metabolism | mitochondria |
| # H-FABP | X14961 | 1.00 | myofiber | energy metabolism | lipid |
| # LCAD | U21489 | 1.00 | myofiber | energy metabolism | lipid |
| # LPL | M60847 | 1.07 | myofiber | energy metabolism | lipid |
| DMPK | Z21503 | 1.04 | myofiber | myogenesis | |
| MEF2C | L13171 | 1.11 | myofiber | myogenesis | |
| MGF | NM_010512 | 1.22 | myofiber | myogenesis | |
| IGF-BP2 | X81580 | 1.04 | myogenesis | | |
| IGF-BP6 | X81584 | 1.26 | myogenesis | | |
| IGF-I | X04480 | 1.21 | myogenesis | | |
| IGF-II | M14951 | 2.32 | myogenesis | | |
| cdk4 | L01640 | 1.52 | proliferation | | |
| c-jun | J04115 | 1.09 | proliferation | | |
| cyclin A1 | X84311 | 1.81 | proliferation | | |
| DNA polymerase d 1 | Z21848 | 1.04 | proliferation | | |
| DNA polymerase e 2 | AF036898 | 1.30 | proliferation | | |
| EGF | J00380 | 1.09 | proliferation | | |
| fra-2 | X83971 | 1.12 | proliferation | | |
| HGF | X72307 | 1.28 | proliferation | | |
| p19 | U19597 | 1.17 | proliferation | | |
| Topoisomerase 1 | D10061 | 1.04 | proliferation | | |
| FGF R2 | M86441 | 1.06 | proliferation | angiogenesis | |
| Follistatin | Z29532 | 1.99 | proliferation | angiogenesis | |
| Ang | U22516 | 1.72 | angiogenesis | | |
| Ang 2 | AF004326 | 1.28 | angiogenesis | | |
| Ang 4 | AF113707 | 1.09 | angiogenesis | | |
| Angrp | U22519 | 1.09 | angiogenesis | | |
| # cadherin 5 | X83930 | 1.10 | angiogenesis | | |
| # CD31 | L06039 | 1.23 | angiogenesis | | |
| plasminogen | J04766 | 1.01 | angiogenesis | wound healing | |
| # VEGF | M95200 | 1.14 | angiogenesis | | |
| VEGF-B | U43836 | 1.00 | angiogenesis | | |
| VEGF-D | D89628 | 1.24 | angiogenesis | | |
| VEGF-R1 | X78568 | 1.16 | angiogenesis | | |
| VEGF-R3 | L07296 | 1.02 | angiogenesis | | |

Table 6 shows gene transcripts in m. solei of one-year-old mice showing a significant genotype difference and belonging to a gene ontology (GO) which was significantly enriched. Expression ratios between TNC-deficient and wildtype mice and GO categories used for testing of enrichment are given. Transcripts of previously identified TNC-pathways are printed in bold. Underlined transcripts were significantly altered in the same direction in m. solei between TNC-deficient and wildtype mice at two-years of age. #, associated with slow-type fibers (Bozyczko D, Decker C, Muschler J & Horwitz A F (1989) Exp Cell Res. 183, 72-91; Bass A, Brdiczka D, Eyer P, Hofer S & Pette D. (1969) Eur. J. Biochem. 10, 198-206; Fluck M. Hoppeler H. (2003) Rev. Physiol Biochem. Pharmacol. 146, 159-216).

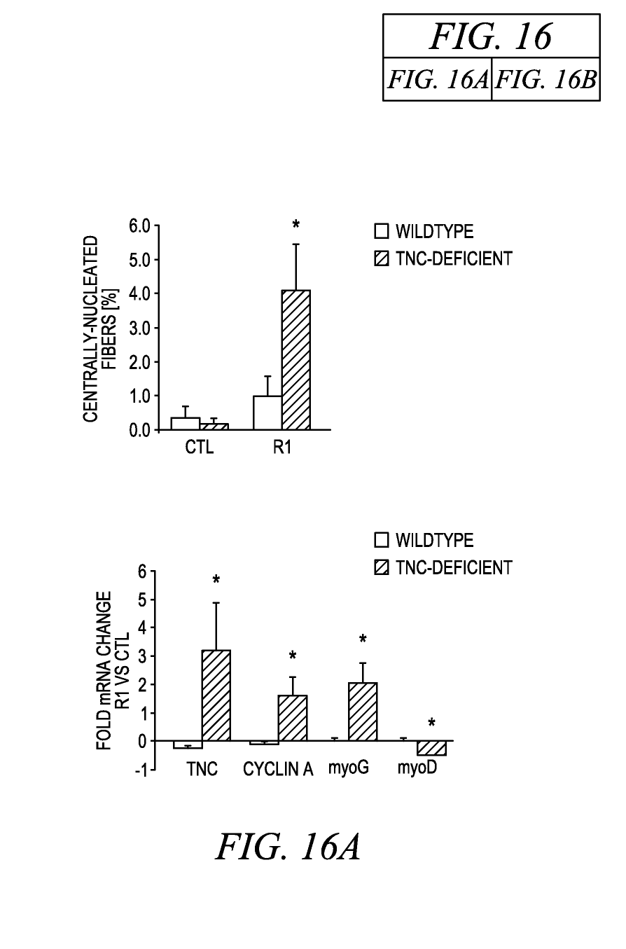

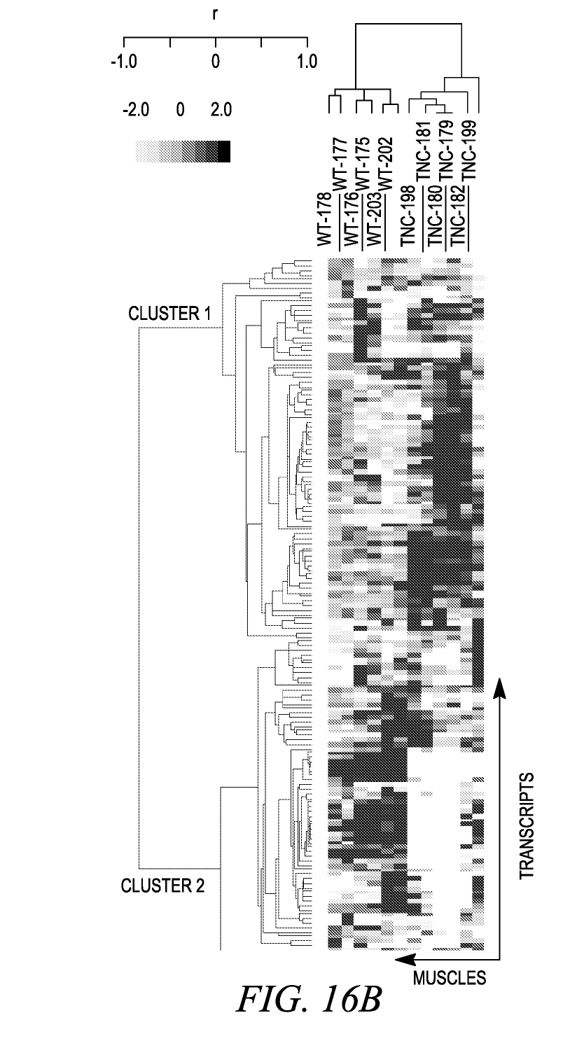

TNC-related molecular response to reloading—Whether altered mechanoresponsiveness of gene expression relates to fast soleus muscle fiber atrophy in one-year old TNC-deficient mice was investigated. Soleus muscle was mechanically challenged by the gravitational stimulus of reloading after deconditioning by 7 days of hindlimb suspension. The deconditioned and reloaded soleus muscle of TNC-deficient mice showed fiber damage which distinguished to cage controls (FIG. 16A). In wildtype mice, no difference was noted between reloaded and cage control muscle (p=0.4).

155 transcripts showed a significant TNC-genotype dependency of the one-day reloading response in one-year-old mice (FIG. 16B, table 7). Multicorrelation testing identified two main clusters of co-regulated mRNA levels. These could be assigned to discrete gene ontologies. Within the cluster of co-incidentally upregulated RNAs, transcripts assigned to de-adhesion, angiogenesis and wound healing were enriched (table 8). Conversely, factors associated with myofibers ‘concentrated’ in the cluster of down-regulated RNAs; the main exception being three up-regulated myogenic regulators myoG, SRF and MEF2A.

TABLE 7
Reloading induced transcript level alterations in m. solei
of wildtype and TNC-deficient mice

R1 v CTL

| Gene | Genbank | TNC- | WT |
|---|---|---|---|
| 14-3-3 zeta/delta | D78647 | 2.21 | 1.09 |
| ACE | J04946 | 1.90 | 1.02 |
| ADAM 2 | U16242 | 2.60 | 0.88 |
| ADMR | D17292 | 2.77 | 0.99 |
| ADORA1 | U05671 | 3.16 | 0.94 |
| ADORA2 | U05672 | 3.20 | 0.96 |
| ADORA2b | U05673 | 2.98 | 1.00 |
| ADORA3 | L20331 | 3.03 | 1.00 |
| ADRA1B | Y12738 | 3.00 | 0.97 |
| AGT | AF045887 | 2.45 | 1.02 |
| AGTR2 | L32840 | 2.77 | 0.88 |
| AhR | D38417 | 2.88 | 0.93 |
| ALDO A | Y00516 | 0.81 | 1.34 |
| ALDO B | X53402 | 2.94 | 0.87 |
| Ang 1 | U83509 | 1.99 | 0.74 |
| Ang 1 R | S67051 | 2.70 | 0.94 |
| Angrp | U22519 | 1.95 | 0.83 |
| Bax | L22472 | 2.31 | 1.18 |
| BTK | NM_013482 | 2.44 | 0.87 |
| Capn3 | X92523 | 2.11 | 0.81 |
| CAST | X62519 | 2.46 | 1.07 |
| cathepsin H | U06119 | 2.26 | 0.93 |
| CD31 | L06039 | 0.25 | 1.08 |
| CD44 | M27129 | 2.36 | 1.09 |
| cdk4 | L01640 | 1.94 | 0.92 |
| c-jun | J04115 | 1.77 | 1.06 |
| COX IVa | X54691 | 0.59 | 0.84 |
| COX Vb | X53157 | 0.62 | 0.91 |
| CSF2Ra | M85078 | 2.68 | 0.96 |
| CSF2Rb | M34397 | 1.83 | 0.89 |
| CSF3R | M58288 | 2.64 | 0.92 |
| c-src | U05247 | 2.58 | 0.94 |
| cyclin A2 | Z26580 | 2.62 | 0.92 |
| cyclin D1 | S78355 | 2.41 | 0.98 |
| Cyt C | X55771 | 2.39 | 0.77 |
| DGAT | AF078752 | 4.10 | 0.86 |
| EGF | J00380 | 1.99 | 0.88 |
| EGF-R | X78987 | 2.05 | 0.81 |
| eNOS 3 | U53142 | 3.13 | 0.89 |
| EphB2 | L25890 | 2.61 | 1.03 |
| EphB3 | Z49086 | 2.45 | 1.00 |
| EphB4 | Z49085 | 2.47 | 1.03 |
| Ephrin B1 | U12983 | 2.38 | 0.90 |
| Ephrin B2 | L38847 | 2.79 | 0.76 |
| EPOR | J04843 | 2.23 | 0.96 |
| ET-1 | U35233 | 2.34 | 0.94 |
| ET-2 | X59556 | 3.01 | 0.95 |
| ET-3 | U32330 | 3.12 | 0.96 |
| FGF R1 | X51893 | 1.68 | 0.85 |
| FGF R3 | X58255 | 4.11 | 0.76 |
| FGF R4 | X59927 | 2.96 | 0.93 |
| FGF-2 | M30644 | 2.79 | 0.96 |
| Fibronectin ED-B | X93167 | 2.98 | 1.02 |
| fra-1 | AF017128 | 2.83 | 0.95 |
| fra-2 | X83971 | 1.88 | 0.90 |
| GAPDH | M32599 | 0.76 | 1.33 |
| GIP | U34295 | 2.47 | 0.93 |
| Glut 2 | X16986 | 1.51 | 0.65 |
| Glut 3 | M75135 | 2.01 | 0.78 |
| Glut 4 | M23383 | 0.51 | 0.80 |
| Hd | U24233 | 2.52 | 0.93 |
| HIF-1a | U59496 | 2.41 | 0.92 |
| HIF-1b | U14333 | 2.00 | 0.84 |
| HK 1 | J05277 | 2.37 | 0.84 |
| HO-1 | M33203 | 2.52 | 1.04 |
| HPRT | J00423 | 3.68 | 0.76 |
| HSL | U08188 | 2.46 | 0.85 |
| IGF I R | AF056187 | 2.48 | 0.86 |
| IGF II R | U04710 | 1.69 | 1.06 |
| IGF-BP1 | X81579 | 3.61 | 0.91 |
| IGF-BP2 | X81580 | 2.04 | 0.81 |
| IGF-BP3 | X81581 | 2.18 | 0.90 |
| IGF-BP4 | X81582 | 2.15 | 1.09 |
| IGF-BP5 | L12447 | 1.68 | 0.80 |
| IGF-BP6 | X81584 | 0.35 | 0.88 |
| IGF-I | X04480 | 0.57 | 0.85 |
| IL1b | M15131 | 2.49 | 0.70 |
| IL6 | X06203 | 3.80 | 0.84 |
| IL6Ra | X51975 | 2.12 | 0.78 |
| InsR | J05149 | 2.23 | 0.76 |
| integrin a2 | X75427 | 3.06 | 0.88 |
| integrin av | U14135 | 2.82 | 0.94 |
| integrin b1 | Y00769 | 1.36 | 1.91 |
| integrin b5 | AF043256 | 1.73 | 0.90 |
| junB | J03236 | 2.83 | 0.63 |
| Lama2 | U12147 | 2.40 | 0.95 |
| Lama3 | X84014 | 0.29 | 1.03 |
| Lamb1 | M15525 | 3.87 | 0.79 |
| Lamb2 | U42624 | 1.71 | 0.90 |
| Lamb3 | U43298 | 2.70 | 0.96 |
| Lamc1 | X05211 | 2.13 | 1.29 |
| Lamc2 | U43327 | 2.58 | 0.97 |
| LDH 1 | U13687 | 1.86 | 0.91 |
| LDH 3 | M17587 | 0.43 | 0.90 |
| LDL R | Z19521 | 2.18 | 0.80 |
| MCAD | U07159 | 1.11 | 0.47 |
| MEF2A | U30823 | 2.16 | 0.98 |
| MEF2B | D50311 | 2.78 | 0.84 |
| MGF | NM_010512 | 0.38 | 1.00 |
| MHC I | AY056464 | 0.39 | 0.78 |
| MHC IIB | AJ278733 | 1.07 | 1.48 |
| MHC IIX | AJ293626 | 0.82 | 1.69 |
| MMP-10 | X76537 | 2.02 | 0.97 |
| MMP-11 | Z12604 | 2.71 | 1.00 |
| MMP-12 | M82831 | 2.46 | 0.83 |
| MMP-14 | X83536 | 2.29 | 0.86 |
| MMP-15 | D86332 | 2.59 | 0.83 |
| MMP-2 | M84324 | 2.11 | 1.02 |
| MMP-3 | X63162 | 3.14 | 0.95 |
| MMP-8 | U96696 | 3.12 | 0.85 |
| MMP-9 | X72795 | 2.66 | 0.86 |
| myf-5 | X56182 | 2.65 | 0.97 |
| myoD | M84918 | 0.54 | 0.99 |
| myoG | D90156 | 3.03 | 1.02 |
| p21 | U09507 | 2.93 | 1.09 |
| PCNA | X53068 | 2.40 | 0.97 |
| PDGFa | M29464 | 3.02 | 0.94 |
| PDGFb | M84453 | 2.77 | 1.02 |
| PDGFRa | M57683 | 2.57 | 0.94 |
| PDGFRb | X04367 | 2.94 | 0.94 |
| PDHA2 | M76728 | 2.37 | 0.92 |
| Pfkfb1 | X98848 | 2.01 | 0.66 |
| Pfkfb2 | X98847 | 2.57 | 0.85 |
| plasminogen | J04766 | 2.55 | 0.93 |
| PIGF | X96793 | 2.95 | 0.97 |
| Pola1 | D17384 | 2.14 | 0.90 |
| PolB | D29013 | 3.03 | 0.89 |
| Pold1 | Z21848 | 1.80 | 0.87 |
| PPARa | X57638 | 1.84 | 0.88 |
| PPARg | U01664 | 2.50 | 0.97 |
| Rpa2 | D00812 | 2.30 | 1.00 |
| RPS29 | L31609 | 1.01 | 1.40 |
| Rps6ka1 | M28489 | 2.80 | 0.90 |
| RPSA | J02870 | 2.64 | 1.13 |
| Scarb2 | AB008553 | 2.10 | 0.77 |
| SRF | NM_020493 | 1.66 | 0.94 |
| Tfam | NM_009360 | 2.50 | 0.96 |
| TGFb1 | M13177 | 2.10 | 1.10 |
| TIMP-1 | X04684 | 2.22 | 0.94 |
| TIMP-2 | X62622 | 1.47 | 0.89 |
| titin | X64700 | 0.57 | 0.84 |
| TNC | D90343 | 4.18 | 0.77 |
| TNW | AJ580920 | 2.94 | 0.99 |
| TOP2a | D12513 | 2.66 | 0.79 |
| TOP3A | AB006074 | 1.87 | 0.82 |
| TOP3b | AB013603 | 2.02 | 0.86 |
| t-PA | J03520 | 2.36 | 0.70 |
| TSP 2 | L07803 | 2.65 | 1.00 |
| TSP 3 | L24434 | 3.02 | 0.98 |
| u-PA | X02389 | 2.54 | 0.96 |
| u-PAR | X62700 | 2.36 | 0.89 |
| VCAM1 | M84487 | 2.93 | 0.93 |
| VEGF | M95200 | 1.70 | 0.86 |
| VEGF-R1 | X78568 | 2.01 | 0.90 |
| VEGF-R2 | X70842 | 2.33 | 0.81 |

Table 7 shows an alphabetically-ordered list of the 155 reloading-induced transcript level alterations in one-year-old mice. Numbers denote the expression ratio of mRNA levels in m. solei after one-day reloaded vs. cage control mice (R1 v CTL) in wildtype (WT) and TNC-deficient mice (TNC-). Transcript alterations which were significant as revealed by SAM are given in bold.

TABLE 8
Genotype differences in transcript expression with reloading of deconditioned soleus muscle.

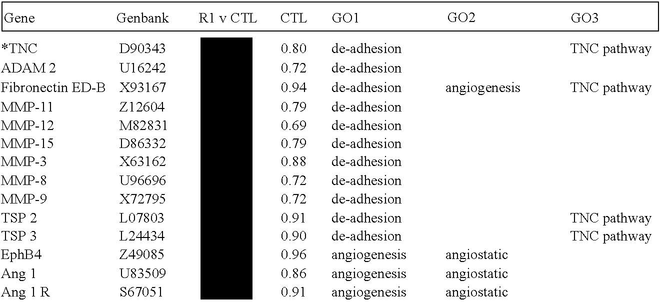

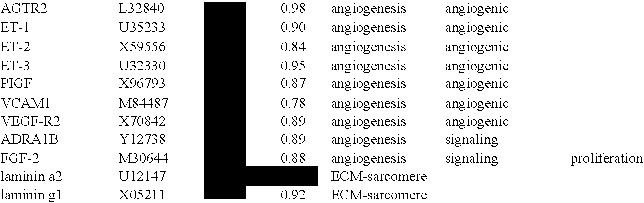

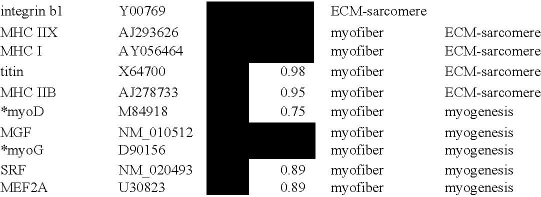

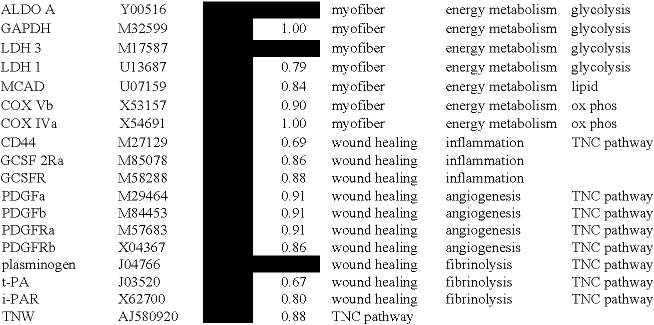

Table 8 shows co-clustered transcripts of enriched GOs which demonstrated a genotype-dependent reloading response in one-year-old mice. Numbers denote genotype ratio of mRNA levels in m. solei of one-day reloaded vs. cage control mice (R1 v CTL) or in cage controls (CTL). Black and grey boxes denote transcripts being significantly up- or down-regulated in TNC-deficient mice in the respective comparison. The three major GOs used for classification are given. Asterisk indicates changes which were verified at the protein level. Gene names in bold reflect TNC genotype-dependent transcripts which expression between genotypes in cage controls was inversed after reloading.

The comparison with cage controls revealed that reloading inversed the transcript expression ratios between genotypes (p=1 E-12, table 8), except for GOs relating to myofibers. This ‘mirror effect’ correlated with the expression of TNC mRNA which was selectively elevated in m. solei of TNC-deficient mice after reloading (FIG. 16C, mean r2=0.92).

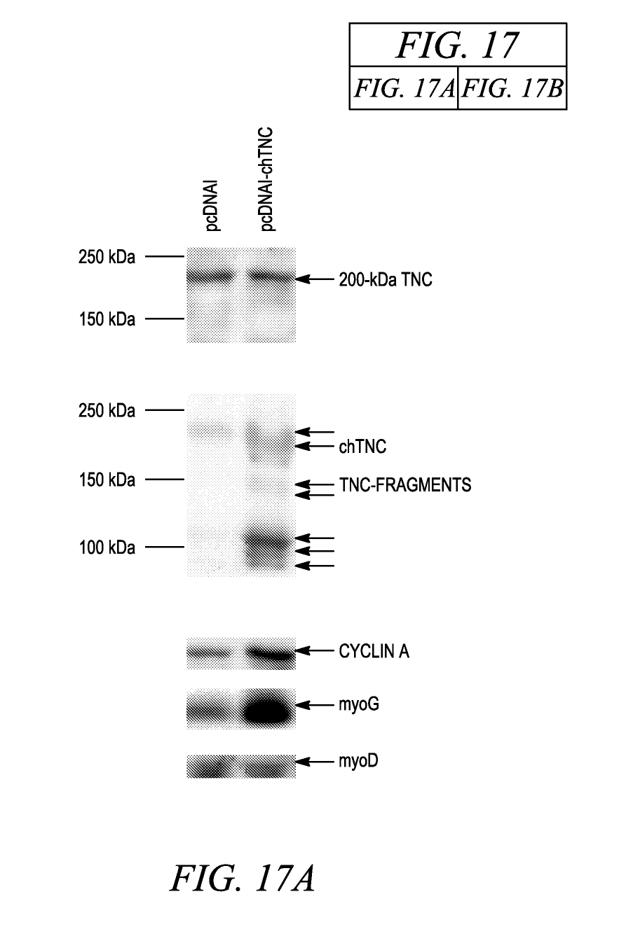

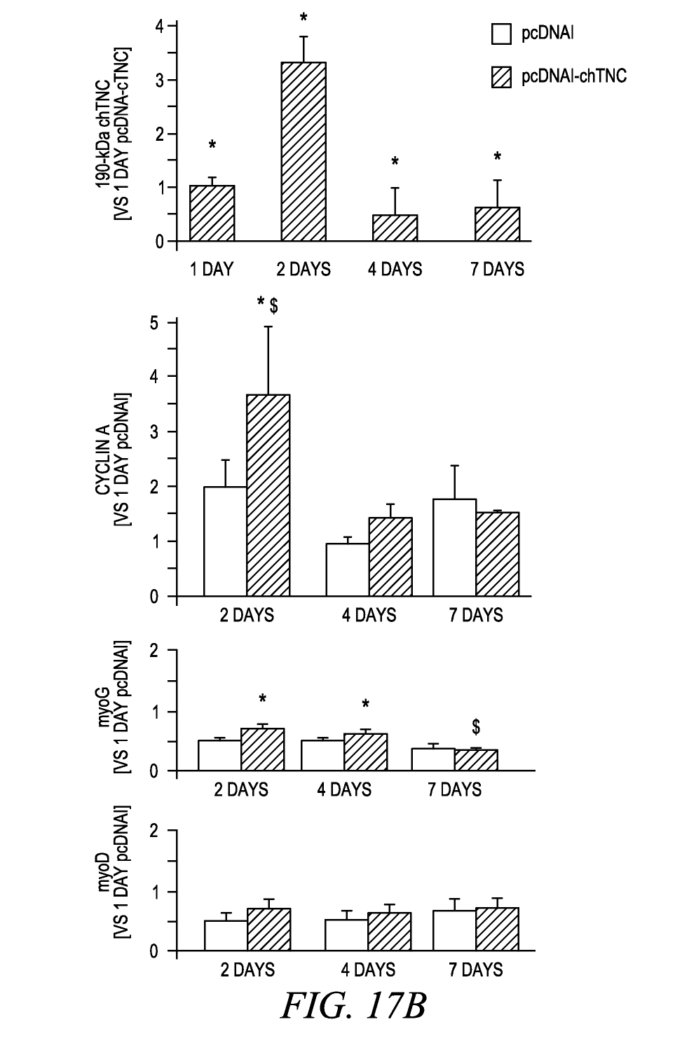

Proof-of-conceit for the myocellular TNC-signaling pathway—Muscle fiber-targeted overexpression of TNC in TNC-deficient mice was carried out to validate the TNC genotype association of gene transcripts at the protein level. The pure fast-type muscle tibialis anterior was chosen to study the TNC-mediated control of selected regulatory factors in relation to fast-type muscle fibers and muscle damage. This concerned the master regulators of myogenesis in slow- and fast-type muscle fibers, myoG and myoD, and the proliferation regulator cyclin A. Reloading ‘inversed’ the expression ratios between genotypes of the two latter transcripts as shown formerly (FIG. 16C). Right muscles were subjected to transfection by electro transfer with a plasmid for constitutive expression of chicken TNC (chTNC). The left muscles, being transfected with empty vector, served as inter-animal specificity control. Muscle damage was visibly induced after electro transfer (data not shown). 190-kDa chTNC (and smaller fragments) were selectively overexpressed in right muscles during the first week after transfection with a maximum after 2 days (FIG. 17A, B). In empty transfected left muscles the 190-kDa chTNC protein was not detected. As well, expression of a 200-kDa TNC related protein was readily detectable on both (muscle) sides.

Quantitative immunoblotting of muscle pairs identified a transient increase of cyclin A and myoG protein levels two days—but not one day—after ‘Knock-In’ (FIG. 17C,D). MyoD protein levels were not significantly affected by TNC-overexpression (FIG. 17E).

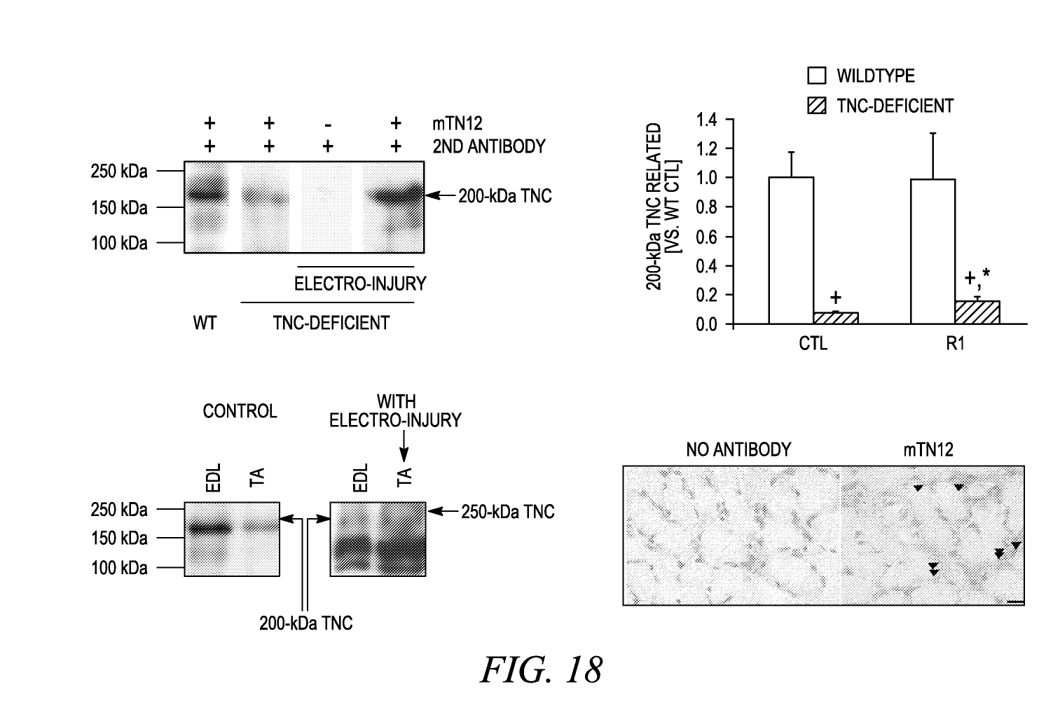

Expression of the small 200-kDa TNC-related protein with muscle damage—TNC-related protein expression in m. tibialis anterior of TNC-deficient mice after electro transfer was investigated. These experiments demonstrated the induced abundance of the 200-kDa TNC-immunoreactive protein in m. tibialis anterior of TNC-deficient mice after electro transfer (FIG. 18A). In wildtype mice, a 3-fold up-regulation of both the large and small TNC isoform was evident after electro transfer (FIG. 18B). Subsequent verification demonstrated the induced expression of the 200-kDa TNC immunoreactive protein in deconditioned soleus muscle of TNC-deficient mice after reloading (FIG. 18C). Microscopic examination of immunostained sections witnessed reloading-induced TNC-staining at the periphery of ˜10% of soleus muscle fibers (FIG. 18D).

### Example 13

### Tenascin-C Exerts Pleiotropic Control Over Muscle Repair

The role of Tenascin-C (TNC) in regenerative processes has been a riddle ever since transgenic mice with targeted ablation of TNC secretion were found to have no obvious phenotype. The present invention sheds light on this matter showing abnormal myogenesis and atrophy of ‘fast-differentiated’ myofibers of locomotor muscles in the original TNC-deficient mouse strain of Faessler. This pathology was related to the blunted expression of the large TNC isoform in TNC-deficient mice and the unexpected expression of a TNC-related protein with muscle fiber damage. The physiological implications are addressed with special emphasis on the novel concept of TNC-mediated coordination of myofiber and extracellular repair processes.

These experiments did not allow for the rejection of the possibility of an atypical TNC protein being produced in the transgenic mouse line under study. Doubt about the absence of TNC in allegedly TNC-deficiency arose with immunoblotting experiments demonstrating induced expression of a 200-kDa protein with muscle reloading and electropulsing of two different leg muscles. This protein was indistinguishable from the small TNC isoform, based on antigenicity and size (see FIG. 18). A functional similarity of this protein in TNC-deficient mice to the small TNC isoform is further suggested by the correlation of enhanced TNC mRNA levels and 200-kDa TNC protein during reloading (FIGS. 16C & 18C) with the inversion of genotype differences of transcripts in cage controls. Western blot experiments excluded the contribution of the related, and similar-sized, Tenascin W isoform to the 200-kDa TNC-related muscle protein (FIG. 22). FIG. 22 shows tenascin-W protein in soleus muscle of wildtype and TNC-deficient mice. A) Representative Western blot. B) Mean and standard error of Tenascin-W protein in muscles of cage controls (CTL) and one day reloaded (R1) animals. This expression of a TNC-related protein in muscle tissue seems to be the consequence of an alternative in-frame start codon in the modified TNC-gene sequence and protein release from damaged cells via a secretion-independent mechanism.

The first supposition is based on DNA-sequencing of the modified TNC-gene. These results identify the presence of a consensus in-frame start codon shortly after the ablated signal peptide which meets the consensus requirements for translation initiation (FIG. 20). The second condition relates to plasma membrane disruptions in cells which reside in tissues that are normally exposed to mechanical stress in vivo.

This damage is an overlooked but common consequence of mechanical loading which would allow direct release of cytoplasmic proteins due to membrane disruption. Both reloading of deconditioned soleus muscle and/or electropulsing provoke damage of the sarcolemma. A possible TNC variant released from damaged muscle of transgenic mice would not be easily distinguishable from the processed muscle-specific 200-kDa TNC isoform (FIGS. 14A & 18B). This is due to the fact that the anticipated site for cleavage of the TNC signal peptide during secretion is only a few kDa away from the alternative start codon (FIG. 20). In contrast, the blunted expression of the 250-kDa TNC protein in leg muscles of the transgenic line after the damaging interventions in the study (compare FIGS. 18A and 18B) suggests that this large TNC isoform relies on active secretion. This conclusion is compatible with the observation that this 250-kDa protein is secreted from interstitial cells. The theoretical and experimental considerations support the notion that possible defects in mechano-sensitive muscle tissue of TNC-deficient mice are masked by release of a short TNC variant upon fiber damage.

TNC-dependent muscle phenotype—This multilevel approach identified a discrete shift of transcript expression in the mixed soleus muscle of TNC-deficient mice towards the characteristics of slow type fibers (table 4). This was accompanied by correspondingly reduced fast fiber volume and slowed contraction (table 4). Also noted as the reduction in fast fiber cross-sectional area in the belly portion of soleus muscle was not matched by differences in muscles mass in one-year old TNC-deficient mice (FIG. 15). This lack of correspondence was not explained by alterations in total muscle fiber number (data not shown). Together with the observation on the elongated tibial bone which defines soleus muscle length (table 4) this indicates a complex role of TNC in determining the ‘architecture’ of the musculoskeletal system which has been overlooked.

Mapping TNC expression targets—The evidence for TNC-dependent gene regulation was corroborated by the transcript response of soleus muscle to reloading. This concerned pronounced elevations of interstitial gene messages in ‘TNC-deficient’ mice when structural and metabolic factors of muscle fibers were down-regulated (supplemental table 5). The genotype-specific up-regulation of mRNAs involved in wound healing, deadhesion and angiogenesis identified a series of novel targets of TNC signaling for the first time in skeletal muscle (table 8). Meanwhile the enhanced gene message of myogenic regulators (myoG, SRF and MEF2A) provided first direct evidence for a role of TNC in myogenesis in vivo.

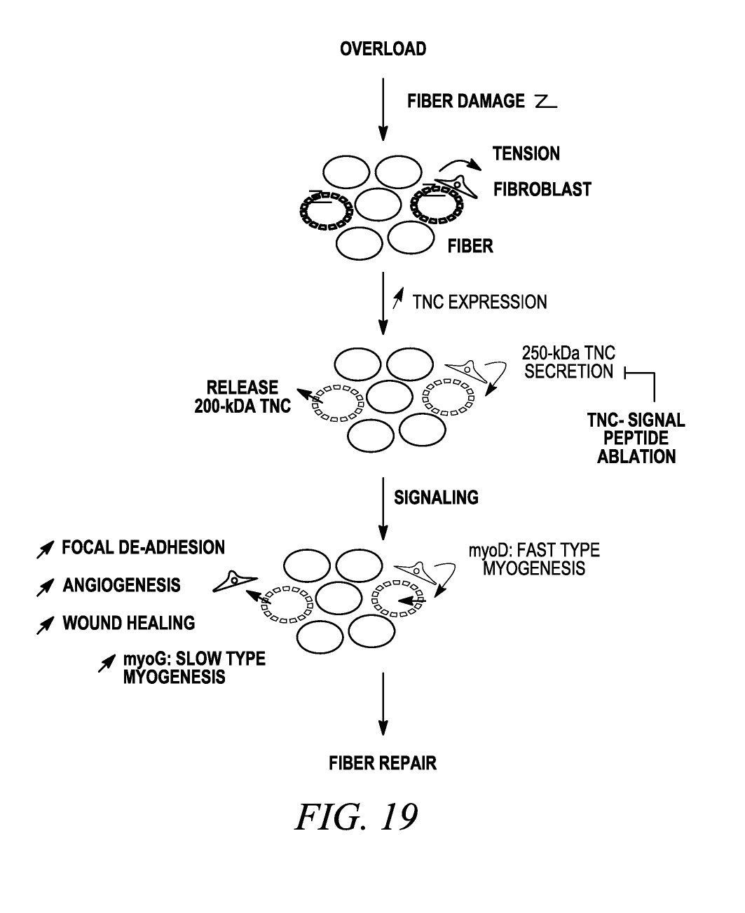

TNC controls ‘slow and fast-type’ myogenesis—The molecular analysis of the reloading response identified that transcript level alterations between TNC-genotypes of the master regulator of slow-type gene expression, myoG, was opposite to the alterations of the fast muscle type-related myoD (table 8, FIG. 16C). The dissimilar association of these myogenic transcription factors with TNC was confirmed at the protein level by muscle fiber-targeted over-expression of chicken TNC in fast-type tibialis anterior muscle (FIG. 17B-E). Whereas myoG protein levels were enhanced after the exogenous TNC overexpression, myoD protein abundance in TNC-deficient mice was not significantly affected (FIG. 17D/E). This suggests that an insufficient level of myoD expression as part of the “fast myogenic program” explains the deterioration of fast-type characteristics of leg muscles in TNC-deficient mice (FIG. 19).

Damage-induced coordination of myocellular and interstitial repair via TNC isoforms—Muscle to loading has been acknowledged earlier to induce a pleiotropic cell response. The functional implication of this complex process was largely ignored until recent microarray studies pointed out a concomitant myocellular and interstitial RNA regulation. The selective increase in fiber damage and TNC expression (FIG. 16A/C & 18C) in TNC-deficient mice with reloading of deconditioned soleus muscle indicates that the concomitantly enhanced interstitial factor expression (table 8) reflects a TNC-mediated damage response. This observation connects the regulation of small and large TNC isoforms to the differential control of slow and fast type myogenesis and cell proliferation. The up-regulation of the small 200-kDa TNC-related protein after muscle fiber damage (by reloading and somatic transgenesis) relates to the promotion of the slow myogenic program via myoG and activated cell proliferation via cyclin A. Conversely, the putative secreted large TNC isoform (which is absent in ‘TNC-deficient’ mice) relates to transcript expression of the ‘fast-type’ myogenic factor myoD (FIG. 19). The present novel results, imply that a novel damage-inducible TNC pathway coordinates the myocellular and interstitial response to mechanical fiber damage.

Mechanically-induced TNC production in muscle and repair—Mechanically-driven expression of the de-adhesive TNC protein is believed to be a requirement for repair of mechanically stressed cells by allowing their relief from strain. This identification of TNC-dependent RNA control of angiogenic, wound healing and certain myogenic factors after the mechanical challenge of reloading provides evidence that this process occurs in striated muscle tissue (table 8). A potential scenario is that these processes are concerted by TNC release from overload damaged cells in conjunction with activated secretion of the large TNC isoform from endomysial fibroblasts subjected to tensile stress (FIG. 19). In this regard, the TNC-promoted enhancement of the major regulators of cell proliferation and differentiation, cyclin A and myoG, in fast-type tibialis anterior muscle (FIG. 17) provides insight into the timing of muscle repair. Their up-regulation after 2 days corresponds to alterations in overloaded chicken muscle and mirrors the retarded cell recruitment following myocardial injury in ‘TNC-deficient’ mice. These observations indicate that damage-induced TNC production governs the pace of muscle fiber repair via the modulation of interstitial and myogenic cell activation at the site of fiber injury.

Cycles of micro-damage and repair have been argued to contribute to basal muscle turnover of skeletal muscles. These observations of the leg muscles of TNC-deficient mice imply a role of load-regulated TNC-upregulation in this damage-repair cycle. The selective alteration of fast muscle type properties in TNC-deficient mice (table 4, FIG. 15) suggests that TNC function relates to contraction-related factors. Interestingly, fast type muscle fibers show preferential vulnerability to reloading damage in rodents, ectopic TNC staining with atrophy and age-induced atrophy in humans (sarcopenia). The structural measures in the anti-gravitation soleus muscle also revealed that defects in TNC protein and associated gene expression accumulate during lifespan to produce a manifestation of fast fiber atrophy at the whole muscle level (FIG. 15A). Collectively these arguments point to deregulated TNC expression as a possible co-factor for the etiology of sarcopenia in humans.

This investigation into the mechano-biology of skeletal muscle show that TNC is part of a pleiotropic pathway that protects fast muscle fiber mass from deleterious consequences of mechanically-induced micro-damage. This insight into the biomechanical control of the muscle phenotype is of relevance for future approaches aiming at reducing or healing musculoskeletal injuries.

### Example 14

### Costameres are Nodal Points of Myofibre Differentiation and Force Transmission In Vivo

Sarcolemmal focal adhesions (costameres) have been hypothesized to integrate the contractile apparatus with the sarcolemma during lengthening and shortening of the muscle cells. Using a system approach this experiment demonstrates the functional implication of costameres in mechano-transduction in fully-differentiated muscle fibres.

Both, muscle-targeted overexpression of the focal adhesion modulators, focal adhesion kinase (FAK) and Tenascin-C, by gene electrotransfer of rodent muscle promoted the slow fibre expression program. Signal transduction to slow muscle transcript expression is under physiological control by muscle loading and functionally important as shown by fast-to-slow transformation of FAK-transfected muscle and fibres. FAK-overexpression also enhanced specific force in whole muscle but not single fibres due to the promotion of repair in damaged fibres after electrotransfer.

The observations support the concept of a double role of costameres in lateral force transmission and chemical mechano-transduction upstream of slow fibre transformation. The load-dependence of costamere's control over muscle function has major implications for effective gene therapy of striated muscle.

### Example 15

Provided hereafter is an example of a representative sequence. The underlined section is the atg start.

### SEQ ID NO: 2 (Tenascin-C)

The plasmid pcDNAI-chTNC used to overexpress Tenascin-C comprised nucleotides 1-5942 from the coding sequence of chicken tenascin C (ACCESSION number M23121) inserted into the pcDNAI cloning vector:

Genbank: ACCESSION number M23121

| | |
|---|---|
| 1 | ggggtttgac aggacggcga ggaatccggg agccgacagc tggctgcagt acctctgctt |
| 61 | cgtggaggct gcccgtggca ggatctgatc cgtcagccca cacgagaata agcgtgccaa |
| 121 | gaaaggaaag gaaactcaac ttagtttgaa ctggctctca aatttctcct ccagtctaca |
| 181 | aaggccaaac aaatataaga ctccatcagc tttgaaaagg aactgagcac tacaatggga |
| 241 | ctcccttccc aggttttggc ctgtgccatc ttaggtttgc tgtaccagca tgccagtggt |
| 301 | gggctcatca agcgaattat ccggcagaag cgggagactg ggctcaatgt gaccttacca |
| 361 | gaggataatc agcctgtggt tttcaatcat gtctacaaca tcaagctgcc tgttggctcc |
| 421 | ctttgctctg tggacctgga cacagcaagc ggggacgcag acctgaaggc agaaattgag |
| 481 | cctgtcaaga attacgagga gcatacggtg aatgagggga accagattgt cttcacgcac |
| 541 | cgcatcaaca ttccccgccg ggcctgtggc tgtgcggctg ccccagacat caaggacctg |
| 601 | ctgagcagac tggaggagct ggaggggctg gtatcctccc tccgggagca gtgtgccagc |
| 661 | ggggctggat gctgtcctaa ttcccagaca gcagaaggtc gcctggacac ggccccctat |
| 721 | tgcagtgggc acggcaacta cagcaccgag atctgtggct gcgtgtgcga gccaggctgg |
| 781 | aaaggcccca actgctccga accggcctgc ccacgcaact gcctcaaccg cggcctctgc |
| 841 | gtgcggggca aatgcatctg cgaggagggc tttaccggcg aggactgcag ccaggctgcc |
| 901 | tgcccgtctg actgcaacga ccaaggcaag tgtgtggatg gggtgtgcgt ctgcttcgag |
| 961 | ggctacacgg gcccggactg cggcgaggag ctctgccccc acgggtgtgg cattcacggg |
| 1021 | cgctgtgtgg gtggacgctg tgtgtgccac gagggcttca ctggcgagga ctgcaatgag |
| 1081 | cccctgtgcc ccaacaactg tcacaaccgc gggcgctgtg tggacaacga gtgcgtctgc |
| 1141 | gatgagggct acacgggaga ggactgcggc gagctgattt gccccaatga ctgctttgac |
| 1201 | cgtgggcgct gtatcaacgg gacctgcttc tgcgaggagg gctacactgg agaggactgc |
| 1261 | ggcgagctga cctgccccaa caactgcaac ggcaacgggc gctgcgagaa cgggctgtgt |
| 1321 | gtgtgccatg agggcttcgt gggggatgac tgcagccaga agaggtgccc gaaggactgc |
| 1381 | aataaccgcg ggcactgcgt ggatgggcgc tgtgtgtgcc atgaggggta cctgggggag |
| 1441 | gactgtgggg agctgcggtg ccccaacgac tgccacaacc gcgggcgctg catcaatggg |
| 1501 | cagtgtgtgt gtgatgaggg attcattggg gaggactgtg gagagctgcg gtgccccaac |
| 1561 | gactgccaca accgcgggcg ctgcgtcaat gggcagtgcg agtgccacga gggattcatc |
| 1621 | ggggaggact gcggggagct gcggtgtccc aacgactgca acagccatgg gcgctgtgtc |
| 1681 | aatgggcagt gcgtgtgtga tgaggggtac acaggggagg actgcgggga gttgcggtgc |
| 1741 | cccaacgact gccacaaccg cgggcgctgc gtggagggac gctgtgtgtg tgacaacggc |
| 1801 | ttcatggggg aggactgcgg ggagctgtcc tgtcccaatg actgccacca gcacgggcgc |
| 1861 | tgcgtcgatg ggcgctgcgt gtgccacgag ggcttcactg gggaagactg ccgggaacgg |
| 1921 | tcctgcccca atgactgcaa caacgtgggc cgctgtgtcg agggacggtg tgtctgtgag |
| 1981 | gaaggttaca tggggatcga ctgttctgat gtgtctcctc caacggagct gactgtaacg |
| 2041 | aatgtaacag ataaaacggt aaatctggaa tggaagcatg agaatctcgt caatgagtac |
| 2101 | cttgtcacct atgtccctac cagcagtggt ggcttagatc tacagttcac cgtaccagga |
| 2161 | aaccagacat ctgccactat tcatgagctg gagcctggtg tggaatactt catccgtgtc |
| 2221 | tttgcaatcc ttaaaaacaa gaaaagtatt ccagtcagtg ccagagtagc gacatatttg |
| 2281 | cctgctccag aaggtctgaa attcaaatct gttagagaaa cgtctgtcca ggtggaatgg |
| 2341 | gatcctctga gcatttcctt tgatggctgg gagctggtct ttcgtaatat gcagaaaaag |
| 2401 | gatgataatg gagacataac cagcagcttg aaaaggccgg agacatcata tatgcagcca |
| 2461 | ggattggcac caggacaaca gtataatgta tcccttcata tagtgaaaaa caataccaga |
| 2521 | ggaccagggc tatcccgagt gataaccaca aaactcgatg cccctagcca gattgaggcg |
| 2581 | aaagatgtca cagacaccac agctctgatc acatggtcca aacccttggc tgaaattgaa |
| 2641 | ggcatagagc tcacatatgg ccccaaggat gttccagggg acaggactac cattgacctc |
| 2701 | tctgaggatg aaaaccaata ttctattgga aacctgaggc cacacacaga atatgaagtg |
| 2761 | acactcattt ctcggcgagg ggacatggag agtgaccctg caaaagaagt ctttgtcaca |
| 2821 | gacttggatg ctccacgaaa cctgaagcga gtgtcacaga cagacaacag cattactttg |
| 2881 | gagtggaaga acagccatgc aaatattgat aattaccgaa ttaagtttgc tcccatttct |
| 2941 | ggtggagacc acactgagct gacagtgcca aagggcaacc aagcaacaac cagagctaca |
| 3001 | ctcacaggtt tgagacctgg aactgaatat ggcattggag tgacagcagt gagacaggac |
| 3061 | agggaaagtg ctcctgctac cattaatgct ggcactgatc ttgataaccc caaggacttg |
| 3121 | gaagtcagtg accccactga aaccaccctg tcccttcgct ggagaagacc agtggccaaa |
| 3181 | tttgatcgtt accgcctcac ttacgttagc ccctctggaa agaagaacga aatggagatc |
| 3241 | cctgtggaca gcacctcttt tatcctgaga ggattagacg cagggacgga gtacaccatc |
| 3301 | agtctagtgg cagagaaagg cagacacaaa agcaaaccca caaccatcaa gggttcgact |
| 3361 | gaggaagaac ctgagcttgg aaacttatca gtgtcagaga ctggctggga tggtttccag |
| 3421 | ctcacctgga cagcagccga cggggcctat gagaactttg tcattcaggt gcagcagtct |
| 3481 | gacaatccag aagaaacctg gaacattaca gtccccggcg gacagcactc tgtgaacgtt |
| 3541 | acaggcctca aggccaacac accttataac gtcacacttt acggtgtgat tcgaggctac |
| 3601 | agaaccaaac ccctttatgt tgaaaccacg acaggagcac accccgaagt tggtgagcta |
| 3661 | accgtttccg acattactcc tgaaagcttc aacctttctt ggacgaccac caacggggac |
| 3721 | tttgacgcct ttactattga aattattgat tctaacaggt tgctggagcc catggagttc |
| 3781 | aacatctcag gcaattcaag aacagctcat atctcagggc tttcccccag cactgatttt |
| 3841 | attgtctacc tctatgggat ctctcatggt ttccgcacac aggcaataag tgctgcggct |
| 3901 | acaacagagg cagaacccga ggtggacaac cttctggttt cagatgctac cccagacggc |
| 3961 | ttccgtctgt cctggactgc agatgatggg gttttcgaca gttttgttct aaaaatcagg |
| 4021 | gataccaaaa ggaaatctga tccactggaa ctcatagtac caggccatga gcgcacccat |
| 4081 | gatataacag ggctgaaaga gggcactgag tatgaaattg agctctatgg agttagcagt |
| 4141 | ggacggcgct cccaacccat aaattcagta gcaaccacag ttgtgggatc tcccaaggga |
| 4201 | atctctttct cggacatcac agaaaactct gctacagtca gctggacacc cccccgcagc |
| 4261 | cgtgtggata gctacagggt ctcctatgtc cccatcacag gcggcactcc caatgttgtt |
| 4321 | acagttgatg gaagcaagac aaggacaaag ctggtgaagt tagtcccagg tgtagactac |
| 4381 | aacgttaata tcatctctgt gaaaggcttt gaagaaagcg aacccatttc tggaattctg |
| 4441 | aaaacagctc tggacagccc gtcaggactg gtagtgatga acattacaga ctcggaggct |
| 4501 | ctggcaacct ggcagcctgc aattgcagct gtggataatt acattgtctc ctactcttct |
| 4561 | gaggatgagc cagaagttac acagatggta tcaggaaaca cagtggagta cgacctgaat |
| 4621 | ggccttcgac ctgcgacaga gtacaccctg agggtgcatg cagtgaagga tgcgcagaag |
| 4681 | agcgagaccc tctccaccca gttcactaca ggactcgatg ctccaaaaga tttaagtgct |
| 4741 | accgaggttc agtcagaaac agctgtgata acgtggaggc ctccacgtgc tcctgtcact |
| 4801 | gattacctcc tgacctacga gtccattgat ggcagagtca aggaagtcat cctagaccct |
| 4861 | gagacgacct cctacaccct gacagagctg agcccatcca ctcaatacac agtgaaactt |
| 4921 | caggcactga gcagatctat gaggagcaaa atgatccaga ctgttttcac cacaactggt |
| 4981 | cttctttatc cttatcctaa agactgctcc caagctctcc tgaatggaga ggtcacctct |
| 5041 | gggctctaca ctatttatct gaatggagac aggacacagc ctctgcaagt cttctgtgac |
| 5101 | atggctgaag atggaggcgg atggattgtg ttcctgaggc gtcaaaatgg aaaggaagat |
| 5161 | ttctacagga actggaagaa ttacgtggcc ggctttggag atcccaagga tgaattctgg |
| 5221 | ataggtctgg agaacctcca caaaatcagc tctcaggggc agtacgagct gcgtgtggat |
| 5281 | ctgagagaca gaggtgagac agcctatgct gtgtacgaca agttcagcgt tggagatgcc |
| 5341 | aagacccggt accggctgag ggtggatggc tacagtggca cagcaggtga ctccatgacc |
| 5401 | taccataatg gaagatcctt ctccactttt gacaaggaca atgattctgc tatcaccaac |
| 5461 | tgtgctttgt catacaaggg tgctttctgg tacaagaatt gtcaccgagt caatctgatg |
| 5521 | ggcagatatg gtgacaacaa ccacagtcag ggtgttaatt ggttccactg gaagggccac |
| 5581 | gaatactcca tccagtttgc agagatgaaa ctgagaccct ccagctttcg gaatctggaa |
| 5641 | ggaagacgaa agcgagcata aagccttggg atggtgaaag ggctacgggc agggcaacat |
| 5701 | ggggagggac agagagcggg gggcatggga ggatctctgg catcactggg gttatgggtg |
| 5761 | tgaggagctg gtagtcgtac caaagcatcg caacccttgg cacaagagcc caaacaacga |
| 5821 | gccttacgtg tcccagcaat tccacagagc agctccagct ctgcccactg ctgatgtcct |
| 5881 | tcacgccaaa gacaacgatc tcaagggttg tatgctgttt tcttcatttt tcttttctca |
| 5941 | gc |

### Example 16

Provided hereafter is an example of a representative sequence.

### SEQ ID NO: 3 (PCDNAI)

Genbank: ACCESSION number IG1047; M59925; M16445; X06296; cloning vector.

ggcgtaatct gctgcttgca aacaaaaaaa ccaccgctac
cagcggtggt ttgtttgccg gatcaagagc taccaactct
ttttccgaag gtaactggct tcagcagagc gcagatacca
aatactgtcc ttctagtgta gccgtagtta ggccaccact
tcaagaactc tgtagcaccg cctacatacc tcgctctgct
aatcctgtta ccagtggctg ctgccagtgg cgataagtcg
tgtcttaccg ggttggactc aagacgatag ttaccggata
aggcgcagcg gtcgggctga acggggggtt cgtgcacaca
gcccagcttg gagcgaacga cctacaccga actgagatac
ctacagcgtg agcattgaga aagcgccacg cttcccgaag
ggagaaaggc ggacaggtat ccggtaagcg gcagggtcgg
aacaggagag cgcacgaggg agcttccagg gggaaacgcc
tggtatcttt atagtcctgt cgggtttcgc cacctctgac
ttgagcgtcg atttttgtga tgctcgtcag gggggcggag
cctatggaaa aacgccagca acgcaagcta gcttctagct
agaaattgta aacgttaata ttttgttaaa attcgcgtta
aatttttgtt aaatcagctc attttttaac caataggccg
aaatcggcaa aatcccttat aaatcaaaag aatagcccga
gatagggttg agtgttgttc cagtttggaa caagagtcca
ctattaaaga acgtggactc caacgtcaaa gggcgaaaaa
ccgtctatca gggcgatggc agaccactac gtgaaccatc
acccaaatca agttttttgg ggtcgaggtg ccgtaaagca
ctaaatcgga accctaaagg gagcccccga tttagagctt
gacggggaaa gccggcgaac gtggcgagaa aggaagggaa
gaaagcgaaa ggagcgggcg ctagggcgct ggcaagtgta
gcggtcacgc tgcgcgtaac caccacaccc gccgcgctta
atgcgccgct acagggcgcg tactatggtt gctttgacga
gaccgtataa cgtgctttcc tcgttggaat cagagcggga
gctaaacagg aggccgatta aagggatttt agacaggaac
ggtacgccag ctggattacc gcggtctttc tcaacgtaac
actttacagc ggcgcgtcat ttgatatgat gcgccccgct
tcccgataag ggagcaggcc agtaaaagca ttacccgtgg
tggggttccc gagcggccaa agggagcaga ctctaaatct
gccgtcatcg acttcgaagg ttcgaatcct tccaccacca
ccatcacttt caaaagtccg aaagaatctg ctccctgctt
gtgtgttgga ggtcgctgag tagtgcgcga gtaaaattta
agctacaaca aggcaaggct tgaccgacaa ttgcatgaag
aatctgctta gggttaggcg ttttgcgctg cttcgcgatg
tacgggccag atatacgcgt tgacattgat tattgactag
ttattaatag taatcaatta cggggtcatt agttcatagc
ccatatatgg agttccgcgt tacataactt acggtaaatg
gcccgcctgg ctgacagaca aacgaccccc gcccattgac
gtcaataatg acgtatgttc ccatagtaac gccaataggg
actttccatt gacgtcaatg ggtggactat ttacggtaaa
ctgcccactt ggcagtacat caagtgtatc atatgccaag
tacgccccct attgacgtca atgacggtaa atggcccgcc
tggcattatg cccagtacat gaccttatgg gactttccta
cttggcagta catctacgta ttagtcatcg ctattaccat
ggtgatgcgg ttttggcagt acatcaatgg gcgtggatag
cggtttgact cacggggatt tccaagtctc caccccattg
acgtcaatgg gagtttgttt tggcaccaaa atcaacggga
ctttccaaaa tgtcgtaaca actccgcccc attgacgcaa
atgggcggta ggcgtgtacg gtgggaggtc tatataagca
gagctctctg gctaactaga gaacccactg cttactggct
tatcgaaatt aatacgactc actataggga gacccaagct
tggtaccgag ctcggatcca ctagtaacgg ccgccagtgt
gctggaattc tgcagatatc catcacactg gcggccgctc
gagcatgcat ctagagggcc ctattctata gtgtcaccta
aatgctagag gatctttgtg aaggaacctt acttctgtgg
tgtgacataa ttggacaaac tacctacaga gatttaaagc
tctaaggtaa atataaaatt tttaagtgta taatgtgtta
aactactgat tctaattgtt tgtgtatttt agattccaac
ctatggaact gatgaatggg agcagtggtg gaatgccttt
aatgaggaaa acctgttttg ctcagaagaa atgccatcta
gtgatgatga ggctactgct gactctcaac attctactcc
tccaaaaaag aagagaaagg tagaagaccc caaggacttt
ccttcagaat tgctaagttt tttgagtcat gctgtgttta
gtaatagaac tcttgcttgc tttgctattt acaccacaaa
ggaaaaagct gcactgctat acaagaaaat tatggaaaaa
tatttgatgt atagtgcctt gactagagat cataatcagc
cataccacat ttgtagaggt tttacttgct ttaaaaaacc
tcccacacct ccccctgaac ctgaaacata aaatgaatgc
aattgttgtt gttaacttgt ttattgcagc ttataatggt
tacaaataaa gcaatagcat cacaaatttc acaaataaag
catttttttc actgcattct agttgtggtt tgtccaaact
catcaatgta tcttatcatg tctggatcat cccgccatgg
tatcaacgcc atatttctat ttacagtagg gacctcttcg
ttgtgtaggt accgctgtat tcctagggaa atagtagagg
caccttgaac tgtctgcatc agccatatag cccccgctgt
tcgacttaca aacacaggca cagtactgac aaacccatac
acctcctctg aaatacccat agttgctagg gctgtctccg
aactcattac accctccaaa gtcagagctg taatttcgcc
atcaagggca gcgagggctt ctccagataa aatagcttct
gccgagagtc ccgtaagggt agacacttca gctaatccct
cgatgaggtc tactagaata gtcagtgcgg ctcccatttt
gaaaattcac ttacttgatc agcttcagaa gatggcggag
ggcctccaac acagtaattt tcctcccgac tcttaaaata
gaaaatgtca agtcagttaa gcaggaagtg gactaactga
cgcagctggc cgtgcgacat cctcttttaa ttagttgcta
ggcaacgccc tccagagggc gtgtggtttt gcaagaggaa
gcaaaagcct ctccacccag gcctagaatg tttccaccca
atcattacta tgacaacagc tgtttttttt agtattaagc
agaggccggg gacccctggg cccgcttact ctggagaaaa
agaagagagg cattgtagag gcttccagag gcaacttgtc
aaaacaggac tgcttctatt tctgtcacac tgtctggccc
tgtcacaagg tcaagcacct ccataccccc tttaataagc
agtttgggaa cgggtgcggg tcttactccg cccatcccgc
ccctaactcc gcccagttcc gcccattctc cgccccatgg
ctgactaatt ttttttattt atgcagaggc cgaggccgcc
tcggcctctg agctattcca gaagtagtga ggaggctttt
ttggaggcct aggcttttgc aaaaagctaa ttc

### Example 17

Tidball J G, Salem G, Zernicke R. Site and mechanical conditions for failure of skeletal muscle in experimental strain injuries. Journal of Applied Physiology 74(3):1280-6, 1993.
Noonan T J, Best T M, Seaber A V, Garrett W E Jr. Identification of a threshold for skeletal muscle injury. American Journal of Sports Medicine 22(2): 257-61, 1994.
Zachary I, Sinnett-Smith J, Rozengurt E, Bombesin, vasopressin, and endothelin stimulation of tyrosine phosphorylation in Swiss 3T3 cells. Identification of a novel tyrosine kinase as a major substrate. Journal of Biological Chemistry 267(27): 19031-4, 1992.
Abedi H, Zachary I. Vascular endothelial growth factor stimulates tyrosine phosphorylation and recruitment to new focal adhesions of focal adhesion kinase and paxillin in endothelial cells. Journal of Biological Chemistry 272(24): 15442-51, 1997.
American Physiological Society. 2002. Guiding principles for research involving animals and human beings. Am J Physiol Regul Integr Comp Physiol 283(2):R281-283.
Bloch R J, Gonzalez-Serratos H. 2003. Lateral force transmission across costameres in skeletal muscle. Exerc Sport Sci Rev 31 (2):73-78.
Bozyczko D, Decker C, Muschler J, Horwitz A F. 1989. Integrin on developing and adult skeletal muscle. Exp Cell Res 183(1):72-91.
Brooks A R, Harkins R N, Wang P, Qian H S, Liu P, Rubanyi G M. 2004. Transcriptional silencing is associated with extensive methylation of the CMV promoter following adenoviral gene delivery to muscle. J Gene Med 6(4):395-404.
Burridge K, Chrzanowska-Wodnicka M. 1996. Focal adhesions, contractility, and signaling. Annu Rev Cell Dev Biol 12:463-518.
Campbell W G, Gordon S E, Carlson C J, Pattison J S, Hamilton M T, Booth F W. 2001. Differential global gene expression in red and white skeletal muscle. Am J Physiol Cell Physiol 280(4):C763-768.
Clemente C F, Corat M A, Saad S T, Franchini K G. 2005. Differentiation of C2C12 myoblasts is critically regulated by FAK signaling. Am J Physiol Regul Integr Comp Physiol 289(3):R862-870.
D'Antona G, Pellegrino M A, Adami R, Rossi R, Carlizzi C N, Canepari M, Saltin B, Bottinelli R. 2003. The effect of ageing and immobilization on structure and function of human skeletal muscle fibres. J Physiol 552(Pt 2):499-511.
Dapp C, Schmutz S, Hoppeler H, Fluck M. 2004. Transcriptional reprogramming and ultrastructure during atrophy and recovery of mouse soleus muscle. Physiol Genomics 20(1):97-107.
Davies P F, Barbee K A, Volin M V, Robotewskyj A, Chen J, Joseph L, Griem M L, Wernick M N, Jacobs E, Polacek D C, dePaola N, Barakat A I. 1997. Spatial relationships in early signaling events of flow-mediated endothelial mechanotransduction. Annu Rev Physiol 59:527-549.
Durieux A C, Bonnefoy R, Busso T, Freyssenet D. 2004. In vivo gene electrotransfer into skeletal muscle: effects of plasmid DNA on the occurrence and extent of muscle damage. J Gene Med 6(7):809-816.
Durieux A C, Bonnefoy R, Manissolle C, Freyssenet D. 2002. High-efficiency gene electrotransfer into skeletal muscle: description and physiological applicability of a new pulse generator. Biochem Biophys Res Commun 296(2):443-450.
Durieux A C, Desplanches D, Freyssenet D, Fluck M. 2007. Mechanotransduction in striated muscle via focal adhesion kinase. Biochem Soc Trans 35(Pt 5):1312-1313.
Erickson H P. 1993. Gene knockouts of c-src, transforming growth factor beta 1, and tenascin suggest superfluous, nonfunctional expression of proteins. J Cell Biol 120(5):1079-1081.
Ervasti J M. 2003. Costameres: the Achilles' heel of Herculean muscle. J Biol Chem 278(16):13591-13594.
Fluck M, Carson J A, Gordon S E, Ziemiecki A, Booth F W. 1999. Focal adhesion proteins FAK and paxillin increase in hypertrophied skeletal muscle. Am J Physiol 277(1 Pt 1):C152-162.
Fluck M, Hoppeler H. 2003. Molecular basis of skeletal muscle plasticity—from gene to form and function. Rev Physiol Biochem Pharmacol 146:159-216.
Fluck M, Schmutz S, Wittwer M, Hoppeler H, Desplanches D. 2005. Transcriptional reprogramming during reloading of atrophied rat soleus muscle. Am J Physiol Regul Integr Comp Physiol 289(1):R4-14.
Fluck M, Ziemiecki A, Billeter R, Muntener M. 2002. Fibre-type specific concentration of focal adhesion kinase at the sarcolemma: influence of fibre innervation and regeneration. J Exp Biol 205(Pt 16):2337-2348.
Fonseca P M, Inoue R Y, Kobarg C B, Crosara-Alberto D P, Kobarg J, Franchini K G. 2005. Targeting to C-terminal myosin heavy chain may explain mechanotransduction involving focal adhesion kinase in cardiac myocytes. Circ Res 96(1):73-81.
Giannone G, Sheetz M P. 2006. Substrate rigidity and force define form through tyrosine phosphatase and kinase pathways. Trends Cell Biol 16(4):213-223.
Giraud M N, Fluck M, Zuppinger C, Suter T M. 2005. Expressional reprogramming of survival pathways in rat cardiocytes by neuregulin-1 beta. J Appl Physiol 99(1):313-322.
Goldspink G. 1999. Changes in muscle mass and phenotype and the expression of autocrine and systemic growth factors by muscle in response to stretch and overload. J Anat 194 (Pt 3):323-334.
Gordon S E, Fluck M, Booth F W. 2001. Selected Contribution: Skeletal muscle focal adhesion kinase, paxillin, and serum response factor are loading dependent. J Appl Physiol 90(3):1174-1183; discussion 1165.
Gronevik E, von Steyern F V, Kalhovde J M, Tjelle T E, Mathiesen I. 2005. Gene expression and immune response kinetics using electroporation-mediated DNA delivery to muscle. J Gene Med 7(2):218-227.
Guan J L. 1997. Role of focal adhesion kinase in integrin signaling. Int J Biochem Cell Biol 29(8-9): 1085-1096.
Habets P E, Franco D, Ruijter J M, Sargeant A J, Pereira J A, Moorman A F. 1999. RNA content differs in slow and fast muscle fibers: implications for interpretation of changes in muscle gene expression. J Histochem Cytochem 47(8):995-1004.
Hamadi A, Bouali M, Dontenwill M, Stoeckel H, Takeda K, Ronde P. 2005. Regulation of focal adhesion dynamics and disassembly by phosphorylation of FAK at tyrosine 397. J Cell Sci 118(Pt 19):4415-4425.
Hecker T P, Gladson C L. 2003. Focal adhesion kinase in cancer. Front Biosci 8:s705-714.
Hildebrand J D, Schaller M D, Parsons J T. 1993. Identification of sequences required for the efficient localization of the focal adhesion kinase, pp 125FAK, to cellular focal adhesions. J Cell Biol 123(4):993-1005.
Hoppeler H, Fluck M. 2003. Plasticity of skeletal muscle mitochondria: structure and function. Med Sci Sports Exerc 35(1):95-104.
Ilic D, Furuta Y, Suda T, Atsumi T, Fujimoto J, Ikawa Y, Yamamoto T, Aizawa S. 1995. Focal adhesion kinase is not essential for in vitro and in vivo differentiation of ES cells. Biochem Biophys Res Commun 209(1):300-309.
Ingber D E. 1997. Tensegrity: the architectural basis of cellular mechanotransduction. Annu Rev Physiol 59:575-599.
Judex S, Zhong N, Squire M E, Ye K, Donahue L R, Hadjiargyrou M, Rubin C T. 2005. Mechanical modulation of molecular signals which regulate anabolic and catabolic activity in bone tissue. J Cell Biochem 94(5):982-994.
Kjaer M. 2004. Role of extracellular matrix in adaptation of tendon and skeletal muscle to mechanical loading. Physiol Rev 84(2):649-698.
Klossner S, Durieux Anne-Cecile, Freyssenet Damien, Flueck Martin An in vivo test system to decipher signaling pathways. invited from Physiological Genomics.
Kovacic-Milivojevic B, Roediger F, Almeida E A, Damsky C H, Gardner D G, Ilic D. 2001. Focal adhesion kinase and p130Cas mediate both sarcomeric organization and activation of genes associated with cardiac myocyte hypertrophy. Mol Biol Cell 12(8):2290-2307.
Li Z, Mericskay M, Agbulut O, Butler-Browne G, Carlsson L, Thornell L E, Babinet C, Paulin D. 1997. Desmin is essential for the tensile strength and integrity of myofibrils but not for myogenic commitment, differentiation, and fusion of skeletal muscle. J Cell Biol 139(1):129-144.
MacKenna D, Summerour S R, Villarreal F J. 2000. Role of mechanical factors in modulating cardiac fibroblast function and extracellular matrix synthesis. Cardiovasc Res 46(2):257-263.
Maniura-Weber K, Goffart S, Garstka H L, Montoya J, Wiesner R J. 2004. Transient overexpression of mitochondrial transcription factor A (TFAM) is sufficient to stimulate mitochondrial DNA transcription, but not sufficient to increase mtDNA copy number in cultured cells. Nucleic Acids Res 32(20):6015-6027.
Mansour H, de Tombe P P, Samarel A M, Russell B. 2004. Restoration of resting sarcomere length after uniaxial static strain is regulated by protein kinase Cepsilon and focal adhesion kinase. Circ Res 94(5):642-649.
Mokhtarian A, Lefaucheur J P, Even P C, Sebille A. 1999. Hindlimb immobilization applied to 21-day-old mdx mice prevents the occurrence of muscle degeneration. J Appl Physiol 86(3):924-931.
Nadruz W, Jr., Corat M A, Marin T M, Guimaraes Pereira G A, Franchini K G. 2005. Focal adhesion kinase mediates MEF2 and c-Jun activation by stretch: Role in the activation of the cardiac hypertrophic genetic program. Cardiovasc Res 68(1):87-97.
Parsons J T. 2003. Focal adhesion kinase: the first ten years. J Cell Sci 116(Pt 8):1409-1416.
Pham C G, Harpf A E, Keller R S, Vu H T, Shai S Y, Loftus J C, Ross R S. 2000. Striated muscle-specific beta(1D)-integrin and FAK are involved in cardiac myocyte hypertrophic response pathway. Am J Physiol Heart Circ Physiol 279(6):H2916-2926.
Pirone D M, Liu W F, Ruiz S A, Gao L, Raghavan S, Lemmon C A, Romer L H, Chen C S. 2006. An inhibitory role for FAK in regulating proliferation: a link between limited adhesion and RhoAROCK signaling. J Cell Biol 174(2):277-288.
Quach N L, Rando T A. 2006. Focal adhesion kinase is essential for costamerogenesis in cultured skeletal muscle cells. Dev Biol 293(1):38-52.
Richardson A, Parsons T. 1996. A mechanism for regulation of the adhesion-associated proteintyrosine kinase pp 125FAK. Nature 380(6574):538-540.
Rizzuto G, Cappelletti M, Maione D, Savino R, Lazzaro D, Costa P, Mathiesen I, Cortese R, Ciliberto G, Laufer R, La Monica N, Fattori E. 1999. Efficient and regulated erythropoietin production by naked DNA injection and muscle electroporation. Proc Natl Acad Sci USA 96(11):6417-6422.
Samarel A M. 2005. Costameres, focal adhesions, and cardiomyocyte mechanotransduction. Am J Physiol Heart Circ Physiol 289(6):H2291-2301.
Sanes J R, Lawrence J C, Jr. 1983. Activity-dependent accumulation of basal lamina by cultured rat myotubes. Dev Biol 97(1):123-136.
Sastry S K, Lakonishok M, Wu S, Truong T Q, Huttenlocher A, Turner C E, Horwitz A F. 1999. Quantitative changes in integrin and focal adhesion signaling regulate myoblast cell cycle withdrawal. J Cell Biol 144(6):1295-1309.
Schlaepfer D D, Mitra S K, Ilic D. 2004. Control of motile and invasive cell phenotypes by focal adhesion kinase. Biochim Biophys Acta 1692(2-3):77-102.
Shyy J Y, Chien S. 1997. Role of integrins in cellular responses to mechanical stress and adhesion. Curr Opin Cell Biol 9(5):707-713.
Taylor J M, Mack C P, Nolan K, Regan C P, Owens G K, Parsons J T. 2001. Selective expression of an endogenous inhibitor of FAK regulates proliferation and migration of vascular smooth muscle cells. Mol Cell Biol 21(5):1565-1572.
Tupling A R, Asahi M, MacLennan D H. 2002. Sarcolipin overexpression in rat slow twitch muscle inhibits sarcoplasmic reticulum Ca2+ uptake and impairs contractile function. J Biol Chem 277(47):44740-44746.
Vandenburgh H H. 1983. Cell shape and growth regulation in skeletal muscle: exogenous versus endogenous factors. J Cell Physiol 116(3):363-371.
Yamada K, Green K G, Samarel A M, Saffitz J E. 2005. Distinct pathways regulate expression of cardiac electrical and mechanical junction proteins in response to stretch. Circ Res 97(4):346-353.
Akimoto K, Nakaya M, Yamanaka T, Tanaka J, Matsuda S, Weng Q P, Avruch J and Ohno S. Atypical protein kinase Clambda binds and regulates p70 S6 kinase. Biochem J 335 (Pt 2): 417-424, 1998.
Andre F and Mir L M. DNA electrotransfer: its principles and an updated review of its therapeutic applications. Gene Ther 11 Suppl 1: S33-S42, 2004.
Baar K and Esser K. Phosphorylation of p70(S6k) correlates with increased skeletal muscle mass following resistance exercise. Am J Physiol 276: C120-C127, 1999.
Baar K, Nader G and Bodine S. Resistance exercise, muscle loading/unloading and the control of muscle mass. Essays in Biochemistry 42: 61-74, 2006.
Bodine S C, Stitt T N, Gonzalez M, Kline W O, Stover G L, Bauerlein R, Zlotchenko E, Scrimgeour A, Lawrence J C, Glass D J and Yancopoulos G D. Akt/mTOR pathway is a crucial regulator of skeletal muscle hypertrophy and can prevent muscle atrophy in vivo. Nat Cell Biol 3: 1014-1019, 2001.
Booth F W, Tseng B S, Fluck M and Carson J A. Molecular and cellular adaptation of muscle in response to physical training. Acta Physiol Scand 162: 343-350, 1998.
Carlson C J, Booth F W and Gordon S E. Skeletal muscle myostatin mRNA expression is fiber-type specific and increases during hindlimb unloading. Am J Physiol 277: R601-R606, 1999.
Cary L A and Guan J L. Focal adhesion kinase in integrin-mediated signaling. Front Biosci 4: D102-D113, 1999.
Chan T O, Rittenhouse S E and Tsichlis P N. AKT/PKB and other D3 phosphoinositide-regulated kinases: kinase activation by phosphoinositide-dependent phosphorylation. Annu Rev Biochem 68: 965-1014, 1999.
Chiquet M and Fluck M. Early responses to mechanical stress: from signals at the cell surface to altered gene expression. In: “Protein Adaptations and Signal Transduction”, K B Storey and J M Storey eds Elsevier Science B.V.: 97-109, 2002.
Cleasby M E, Davey J R, Reinten T A, Graham M W, James D E, Kraegen E W and Cooney G J. Acute bidirectional manipulation of muscle glucose uptake by in vivo electrotransfer of constructs targeting glucose transporter genes. Diabetes 54: 2702-2711, 2005.
Dapp C, Schmutz S, Hoppeler H and Fluck M. Transcriptional reprogramming and ultrastructure during atrophy and recovery of mouse soleus muscle. Physiol Genomics 20: 97-107, 2004.
Dardevet D, Sornet C, Vary T and Grizard J. Phosphatidylinositol 3-kinase and p70 s6 kinase participate in the regulation of protein turnover in skeletal muscle by insulin and insulin-like growth factor I. Endocrinology 137: 4087-4094, 1996.
Dennis P B, Pullen N, Pearson R B, Kozma S C and Thomas G. Phosphorylation sites in the autoinhibitory domain participate in p70(s6k) activation loop phosphorylation. J Biol Chem 273: 14845-14852, 1998.
Devor B B, Zhang X, Patel S K, Polte T R and Hanks S K. Chicken and Mouse Focal Adhesion Kinases Are Similar in Structure at Their Amino Termini. Biochemical and Biophysical Research Communications 190: 1084-1089, 1993.
Durieux A C, Bonnefoy R, Busso T and Freyssenet D. In vivo gene electrotransfer into skeletal muscle: effects of plasmid DNA on the occurrence and extent of muscle damage. J Gene Med 6: 809-816, 2004.
Durieux A C, Bonnefoy R, Manissolle C and Freyssenet D. High-efficiency gene electrotransfer into skeletal muscle: description and physiological applicability of a new pulse generator. Biochem Biophys Res Commun 296: 443-450, 2002.
Durieux A C, Desplanches D, Freyssenet D and Fluck M. Mechanotransduction in striated muscle via focal adhesion kinase. Biochem Soc Trans 35: 1312-1313, 2007.
Flück M, Carson J A, Gordon S E, Ziemiecki A and Booth F W. Focal adhesion proteins FAK and paxillin increase in hypertrophied skeletal muscle. Am J Physiol 277: C152-C162, 1999.
Fluck M, Dapp C, Schmutz S, Wit E and Hoppeler H. Transcriptional profiling of tissue plasticity: role of shifts in gene expression and technical limitations. J Appl Physiol 99: 397-413, 2005.
Fluck M and Hoppeler H. Molecular basis of skeletal muscle plasticity—from gene to form and function. Rev Physiol Biochem Pharmacol 146: 159-216, 2003.
22. Fluck M, Schmutz S, Wittwer M, Hoppeler H and Desplanches D. Transcriptional reprogramming during reloading of atrophied rat soleus muscle. Am J Physiol Regul Integr Comp Physiol 289: R4-14, 2005.
Fluck M, Waxham M N, Hamilton M T and Booth F W. Skeletal muscle Ca(2+)-independent kinase activity increases during either hypertrophy or running. J Appl Physiol 88: 352-358, 2000.
Fluck M, Ziemiecki A, Billeter R and Muntener M. Fibre-type specific concentration of focal adhesion kinase at the sarcolemma: influence of fibre innervation and regeneration. J Exp Biol 205: 2337-2348, 2002.
Gan B, Yoo Y and Guan J L. Association of focal adhesion kinase with tuberous sclerosis complex 2 in the regulation of s6 kinase activation and cell growth. J Biol Chem 281: 37321-37329, 2006.
Gehl J, Sorensen T H, Nielsen K, Raskmark P, Nielsen S L, Skovsgaard T and Mir L M. In vivo electroporation of skeletal muscle: threshold, efficacy and relation to electric field distribution. Biochim Biophys Acta 1428: 233-240, 1999.
Gingras A C, Raught B and Sonenberg N. Regulation of translation initiation by FRAP/mTOR. Genes & Development 15: 807-826, 2001.
Gordon S E, Fluck M and Booth F W. Selected Contribution: Skeletal muscle focal adhesion kinase, paxillin, and serum response factor are loading dependent. J Appl Physiol 90: 1174-1183, 2001.
Guerfali I, Manissolle C, Durieux A C, Bonnefoy R, Bartegi A and Freyssenet D. Calcineurin A and CaMKIV transactivate PGC-1 alpha promoter, but differentially regulate cytochrome c promoter in rat skeletal muscle. Pflugers Arch 454: 297-305, 2007.
Hemmings B A. Akt signaling: linking membrane events to life and death decisions. Science 275: 628-630, 1997.
31. Hunter T. Signaling—2000 and beyond. Cell 100: 113-127, 2000.
Ingber D E. Cellular mechanotransduction: putting all the pieces together again. Faseb J 20: 811-827, 2006.
Isotani S, Hara K, Tokunaga C, Inoue H, Avruch J and Yonezawa K. Immunopurified mammalian target of rapamycin phosphorylates and activates p70 S6 kinase alpha in vitro. J Biol Chem 274: 34493-34498, 1999.
Kimball S R, Horetsky R L and Jefferson L S. Signal transduction pathways involved in the regulation of protein synthesis by insulin in L6 myoblasts. Am J Physiol 274: C221-C228, 1998.
Kimball S R, O'Malley J P, Anthony J C, Crozier S J and Jefferson L S. Assessment of biomarkers of protein anabolism in skeletal muscle during the life span of the rat: sarcopenia despite elevated protein synthesis. American Journal of Physiology-Endocrinology and Metabolism 287: E772-E780, 2004.
Laser M, Kasi V S, Hamawaki M, Cooper G, Kerr C M and Kuppuswamy D. Differential activation of p70 and p85 S6 kinase isoforms during cardiac hypertrophy in the adult mammal. J Biol Chem 273: 24610-24619, 1998.
Malik R K and Parsons J T. Integrin-dependent activation of the p70 ribosomal S6 kinase signaling pathway. J Biol Chem 271: 29785-29791, 1996.
Manning G, Whyte D B, Martinez R, Hunter T and Sudarsanam S. The protein kinase complement of the human genome. Science 298: 1912-1934, 2002.
Mir L M, Moller P H, Andre F and Gehl J. Electric pulse-mediated gene delivery to various animal tissues. Adv Genet. 54: 83-114, 2005.
Napoli R, Gibson L, Hirshman M F, Boppart M D, Dufresne S D, Horton E S and Goodyear L J. Epinephrine and insulin stimulate different mitogen-activated protein kinase signaling pathways in rat skeletal muscle. Diabetes 47: 1549-1554, 1998.
Neumann E, Schaefer-Ridder M, Wang Y and Hofschneider P H. Gene transfer into mouse lyoma cells by electroporation in high electric fields. EMBO J 1: 841-845, 1982.
Pallafacchina G, Calabria E, Serrano A L, Kalhovde J M and Schiaffino S. A protein kinase B-dependent and rapamycin-sensitive pathway controls skeletal muscle growth but not fiber type specification. Proc Natl Acad Sci USA 99: 9213-9218, 2002.
Pullen N and Thomas G. The modular phosphorylation and activation of p70s6k. FEBS Lett 410: 78-82, 1997.
Reynolds T H, Bodine S C and Lawrence J C, Jr. Control of Ser2448 phosphorylation in the mammalian target of rapamycin by insulin and skeletal muscle load. J Biol Chem 277: 17657-17662, 2002.
Rickert D, Lendlein A, Peters I, Moses M A and Franke R P. Biocompatibility testing of novel multifunctional polymeric biomaterials for tissue engineering applications in head and neck surgery: an overview. European Archives of Oto-Rhino-Laryngology 263: 215-222, 2006.
Sale E M, Atkinson P P, Arnott C H, Chad J E and Sale G J. Role of ERK1/ERK2 and p70S6K pathway in insulin signalling of protein synthesis. FEBS Lett 446: 122-126, 1999.
Spangenburg E E, LeRoith D, Ward C W and Bodine S. A functional insulin-like growth factor receptor is not necessary for load-induced skeletal muscle hypertrophy. Journal of Physiology 586: 283-291, 2008.
Thomas G and Hall M N. TOR signalling and control of cell growth. Curr Opin Cell Biol 9: 782-787, 1997.
Thomason D B and Booth F W. Stable incorporation of a bacterial gene into adult rat skeletal muscle in vivo. Am J Physiol 258: C578-C581, 1990.
Thomson D M and Gordon S E. Impaired overload-induced muscle growth is associated with diminished translational signalling in aged rat fast-twitch skeletal muscle. J Physiol 574: 291-305, 2006.
Trollet C, Bloquel C, Scherman D and Bigey P. Electrotransfer into skeletal muscle for protein expression. Curr Gene Ther 6: 561-578, 2006.
Potter H, Wier L, Leder P. Enhancer-dependent expression of human kappa immunoglobulin genes introduced into mouse pre-B lymphocytes by electroporation. Proc Natl Acad Sci USA. 81(22): 7161-5, 1984.
Tur-Kaspa R, Teicher L, Levine B J, Skoultchi A I, and Shafritz D A, Use of electroporation to introduce biologically active foreign genes into primary rat hepatocytes. Mol Cell Biol 6(2):716-8, 1986
Chiquet-Ehrismann, R. & Chiquet, M. (2003) J Pathol. 200, 488-499.
Imanaka-Yoshida, K., Hiroe, M., Yasutomi, Y., Toyozaki, T., Tsuchiya, T., Noda, N., Maki, T., Nishikawa, T., Sakakura, T. & Yoshida, T. (2002) J Pathol. 197, 388-394.
Aufderheide, E., Chiquet-Ehrismann, R. & Ekblom, P. (1987) J Cell Biol 105, 599-608.
Thesleff, I., Vaahtokari, A., Kettunen, P. & Aberg, T. (1995) Connect Tissue Res 32, 9-15.
Hurme, T. & Kalimo, H. (1992) Muscle Nerve 15, 482-489.
Fluck, M., Tunc-Civelek, V. & Chiquet, M. (2000) J Cell Sci 113 (Pt 20), 3583-3591.
Fluck, M., Chiquet, M., Schmutz, S., Mayet-Sornay, M. H. & Desplanches, D. (2003) Am J Physiol Regul. ol 284, R792-R801.
Chen, Y. W., Hubal, M. J., Hoffman, E. P., Thompson, P. D. & Clarkson, P. M. (2003) J Appl Physiol 95, 2485-2494.
Crameri, R. M., Langberg, H., Teisner, B., Magnusson, P., Schroder, H. D., Olesen, J. L., Jensen, C. H., Koskinen, S., Suetta, C. & Kjaer, M. (2004) Matrix Biol 23, 259-264.
Jarvinen, T. A., Jozsa, L., Kannus, P., Jarvinen, T. L., Hurme, T., Kvist, M., Pelto-Huikko, M., Kalimo, H. & Jarvinen, M. (2003) J Cell Sci 116, 857-866.
Chiquet, M. & Fluck, M. (2003) J Cell Sci 116, 3851-3853.
Roth-Kleiner, M., Hirsch, E. & Schittny, J. C. (2004) Am J Respir Cell Mol. Biol 30, 360-366.
Murphy-Ullrich, J. E. (2001) J Clin Invest 107, 785-790.
Fischer, D., Tucker, R. P., Chiquet-Ehrismann, R. & Adams, J. C. (1997) Mol. Biol Cell 8, 2055-2075.
Mackie, E. J. & Tucker, R. P. (1999) J Cell Sci 112 (Pt 22), 3847-3853.
Wenk, M. B., Midwood, K. S. & Schwarzbauer, J. E. (2000) J Cell Biol 150, 913-920.
Irintchev, A., Salvini, T. F., Faissner, A. & Wernig, A. (1993) J Neurocytol 22, 955-965.
Fluck, M., Schmutz, S., Wittwer, M., Hoppeler, H. & Desplanches, D. (2005) Am J Physiol 289, R4-14.
Erickson, H. P. (1993) J Cell Biol 120, 1079-1081.
Forsberg, E., Hirsch, E., Frohlich, L., Meyer, M., Ekblom, P., Aszodi, A., Werner, S. & Fassler, R. (1996) Proc Natl Acad Sci USA 93, 6594-6599.
Saga, Y., Yagi, T., Ikawa, Y., Sakakura, T. & Aizawa, S. (1992) Genes Dev. 6, 1821-1831.
Tamaoki, M., Imanaka-Yoshida, K., Yokoyama, K., Nishioka, T., Inada, H., Hiroe, M., Sakakura, T. & Yoshida, T. (2005) Am J Pathol 167, 71-80.
Ballard, V. L., Sharma, A., Duignan, I., Holm, J. M., Chin, A., Choi, R., Hajjar, K. A., Wong, S. C. & Edelberg, J. M. (2006) FASEB J 20, 717-719.
Cifuentes-Diaz, C., Faille, L., Goudou, D., Schachner, M., Rieger, F. & Angaut-Petit, D. (2002) J Neurosci. Res. 67, 93-99.
Matsuda, A., Yoshiki, A., Tagawa, Y., Matsuda, H. & Kusakabe, M. (1999) Invest Opthalmol Vis Sci 40, 1071-1080.
Mitrovic, N. & Schachner, M. (1995) J Neurosci Res 42, 710-717.
Settles, D. L., Kusakabe, M., Steindler, D. A., Fillmore, H. & Erickson, H. P. (1997) J Neurosci Res 47, 109-117.
Redick, S. D. & Schwarzbauer, J. E. (1995) J Cell Sci 108 (Pt 4), 1761-1769.
Kozak, M. (1996) Mamm Genome 7, 563-574.
McNeil, P. L. & Khakee, R. (1992) Am J Pathol 140, 1097-1109.
Booth, F. W. & Thomason, D. B. (1991) Physiol Rev 71, 541-585.
Dapp, C., Schmutz, S., Hoppeler, H. & Fluck, M. (2004) Physiol Genomics 20, 97-107.
Durieux, A. C., Bonnefoy, R., Manissolle, C. & Freyssenet, D. (2002) Biochem Biophys Res Commun 296, 443-450.
Fluck, M. & Hoppeler, H. (2003) Rev. Physiol Biochem Pharmacol 146, 159-216.
Tidball, J. G., Berchenko, E. & Frenette, J. (1999) J Leukoc. Biol 65, 492-498.
Fischer, D., Brown-Ludi, M., Schulthess, T. & Chiquet-Ehrismann, R. (1997) J Cell Sci 110 (Pt 13), 1513-1522.
Aufderheide, E. & Ekblom, P. (1988) J Cell Biol 107, 2341-2349.
Chiquet, M., Vrucinic-Filipi, N., Schenk, S., Beck, K. & Chiquet-Ehrismann, R. (1991) Eur J Biochem 199, 379-388.
Scherberich, A., Tucker, R. P., Degen, M., Brown-Luedi, M., Andres, A. C. &
Chiquet-Ehrismann, R. (2005) Oncogene 24, 1525-1532.
Anon. (2002) Am J Physiol Regul 283, R281-R283.
Krippendorf, B. B. & Riley, D. A. (1993) Muscle Nerve 16, 99-108.
Fluck, M., Ziemiecki, A., Billeter, R. & Muntener, M. (2002) J Exp Biol 205, 2337-2348.
Giraud, M. N., Fluck, M., Zuppinger, C. & Suter, T. M. (2005) J Appl Physiol 99, 313-322.
Humbert, D., Cruz-Orive, L M, Weibel, E R, Gehr, P., Burri, P. & Hoppeler, H. (1990) Acta Stereol 9, 111-124.
Andrade, F. H., Hatala, D. A. & McMullen, C. A. (2004) Pflugers Arch 448, 547-551.
Armstrong, R. B. & Phelps, R. O. (1984) Am J Anat 171, 259-272.
Sakuma, K., Watanabe, K., Sano, M., Uramoto, I., Sakamoto, K. & Totsuka, T. (1999) Biochim Biophys Acta 1428, 284-292.
Fluck, M., Kitzmann, M., Dapp, C., Chiquet, M., Booth, F. W. & Fernandez, A. (2003) J Appl Physiol 95, 1664-1671.
Aszodi, A., Bateman, J. F., Gustafsson, E., Boot-Handford, R. & Fassler, R. (2000) Cell Struct Funct. 25, 73-84.
Muesch, A., Hartmann, E., Rohde, K., Rubartelli, A., Sitia, R. & Rapoport, T. A. (1990) Trends Biochem Sci 15, 86-88.
Clarke, M. S., Vanderburg, C. R. & Feeback, D. L. (2001) J Gravit Physiol 8, 37-47.
Gissel, H. & Clausen, T. (2003) Am J Physiol 285, R132-R142.
Jones, P. L., Crack, J. & Rabinovitch, M. (1997) J Cell Biol 139, 279-293.
Preissner, K. T., May, A. E., Wohn, K. D., Germer, M. & Kanse, S. M. (1997) Thromb Haemost 78, 88-95.
Matsumoto, K., Hiraiwa, N., Yoshiki, A., Ohnishi, M. & Kusakabe, M. (2002) Biochem Biophys Res Commun 290, 1220-1227.
Garcion, E., Faissner, A. & ffrench-Constant, C. (2001) Development 128, 2485-2496.
Gundersen, D., Tran-Thang, C., Sordat, B., Mourali, F. & Ruegg, C. (1997) J Immunol. 158, 1051-1060.
Hughes, S. M., Taylor, J. M., Tapscott, S. J., Gurley, C. M., Carter, W. J. & Peterson, C. A. (1993) Development 118, 1137-1147.
Jablecki, C. K., Heuser, J. E. & Kaufman, S. (1973) J Cell Biol 57, 743-759.
Laurent, G. J., Sparrow, M. P. & Millward, D. J. (1978) Biochem J 176, 407-417.
Pattison, J. S., Folk, L. C., Madsen, R. W., Childs, T. E. & Booth, F. W. (2003) Physiol Genomics 15, 34-43.
Carson, J. A., Nettleton, D. & Reecy, J. M. (2002) FASEB J 16, 207-209.
Lowe, D. A. & Alway, S. E. (1999) Cell Tissue Res 296, 531-539.
Charge, S. B. & Rudnicki, M. A. (2004) Physiol Rev. 84, 209-238.
Vijayan, K., Thompson, J. L., Norenberg, K. M., Fitts, R. H. & Riley, D. A. (2001) J Appl Physiol 90, 770-776.
Schoser, B. G., Faissner, A. & Goebel, H. H. (1999) J Mol Neurosci 13, 167-175.

### B. EXAMPLES OF EMBODIMENTS

Described hereafter are non-limiting examples of embodiments of the invention.

1. A method for improving muscle function in a subject, which comprises:
enhancing focal adhesion signaling in a muscle of a subject; and
providing a load to the muscle;
whereby the focal adhesion signaling is enhanced and the load is provided each in an amount effective to improve the function of the muscle.

2. The method of claim 1, wherein the focal adhesion signaling is enhanced by administering a signaling pathway agonist.

3. The method of claim 1, wherein the focal adhesion signaling is enhanced by administering a signaling pathway member.

4. The method of claim 2, wherein the agonist is a pharmacological drug selected from the group consisting of bombesin, vasopressin, endothelin, vascular endothelial growth factor, angiotensin 2, activators of integrin signaling, activators of G-protein signaling, and reactive oxygen species

5. The method of claim 3, wherein the signaling pathway member is administered by delivering a nucleic acid that encodes the member.

6. The method of claim 5, wherein the nucleic acid encoding the member comprises a vector, a plasmid, or a recombinant viral vector.

7. The method of claim 6, wherein the nucleic acid is operably linked to a control element capable of directing in vivo transcription of the nucleic acid.

8. The method of claim 3, wherein the signaling pathway member is administered by delivering a protein that encodes the member.

9. The method of claim 3, wherein the signaling pathway member is selected from the group consisting of focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

10. The method of claim 9, wherein the focal adhesion kinase (FAK) sequence is identical to or substantially identical to a fragment of an amino acid sequence encoded by SEQ ID No. 1.

11. The method of claim 3, wherein the signaling pathway member comprises a detectable tag.

12. The method of claim 11, wherein the tag is selected from the group consisting of an epitope tag, a fluorescent tag, an affinity tag, a solubilization tag, and a chromatography tag.

13. The method of claims 2 or 3, wherein the signaling pathway agonist or member are administered to the subject from the group consisting of oral, rectal, transmucosal, transdermal, pulmonary, ophthalmic, intestinal, intramuscular, subcutaneous, intravenous, intramedullary, intrathecal, direct intraventricular, intraperitoneal, intranasal, and intraocular means.

14. The method of claim 1, wherein the focal adhesion signaling is enhanced after load is provided to the muscle.

15. The method of claim 1, wherein the load is provided to the muscle after focal adhesion signaling is enhanced.

16. The method of claim 1, wherein the muscle is selected from the group comprising skeletal, cardiac, smooth, slow oxidative fibers, fast oxidative fibers and fast glycolytic fibers.

17. A method for determining whether a subject will respond to a treatment for improving muscle function, which comprises:
measuring the activity of a focal adhesion signaling pathway member in a sample from a subject who has undergone or will undergo a treatment for muscle function that comprises (i) enhancing focal adhesion signaling in a muscle of a subject; and (ii) providing a load to the muscle; and
determining whether the subject will respond to the treatment based on the measured activity.

18. The method of claim 17, the focal adhesion signaling pathway member is selected from the group consisting of focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

19. The method of claim 18, wherein the S6 kinase activity measures the amount of S6 kinase RNA.

20. The method of claim 18, wherein the S6 kinase activity measures the amount of S6 kinase protein.

21. The method of claim 18, wherein the S6 kinase activity measures the degree of S6 kinase phosphorylation.

22. The method of claim 18, wherein the S6 kinase activity measures the phosphotransfer activity of S6 kinase.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method for improving muscle function in a subject, which comprises:
enhancing focal adhesion signaling in a muscle of a subject; and
providing a load to the muscle;
whereby the focal adhesion signaling is enhanced and the load is provided each in an amount effective to improve the function of the muscle.

2. The method of claim 1, wherein the focal adhesion signaling is enhanced by administering a signaling pathway agonist.

3. The method of claim 1, wherein the focal adhesion signaling is enhanced by administering a signaling pathway member.

4. The method of claim 2, wherein the agonist is a pharmacological drug selected from the group consisting of bombesin, vasopressin, endothelin, vascular endothelial growth factor, angiotensin 2, activators of integrin signaling, activators of G-protein signaling, and reactive oxygen species

5. The method of claim 3, wherein the signaling pathway member is administered by delivering a nucleic acid that encodes the member.

6. The method of claim 5, wherein the nucleic acid encoding the member comprises a vector, a plasmid, or a recombinant viral vector.

7. The method of claim 6, wherein the nucleic acid is operably linked to a control element capable of directing in vivo transcription of the nucleic acid.

8. The method of claim 3, wherein the signaling pathway member is administered by delivering a protein that encodes the member.

9. The method of claim 3, wherein the signaling pathway member is selected from the group consisting of focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

10. The method of claim 9, wherein the focal adhesion kinase (FAK) sequence is identical to or substantially identical to a fragment of an amino acid sequence encoded by SEQ ID No. 1.

11. The method of claim 3, wherein the signaling pathway member comprises a detectable tag.

12. The method of claim 11, wherein the tag is selected from the group consisting of an epitope tag, a fluorescent tag, an affinity tag, a solubilization tag, and a chromatography tag.

13. The method of claim 3, wherein the signaling pathway agonist or member are administered to the subject from the group consisting of oral, rectal, transmucosal, transdermal, pulmonary, ophthalmic, intestinal, intramuscular, subcutaneous, intravenous, intramedullary, intrathecal, direct intraventricular, intraperitoneal, intranasal, and intraocular means.

14. The method of claim 1, wherein the focal adhesion signaling is enhanced after load is provided to the muscle.

15. The method of claim 1, wherein the load is provided to the muscle after focal adhesion signaling is enhanced.

16. The method of claim 1, wherein the muscle is selected from the group comprising skeletal, cardiac, smooth, slow oxidative fibers, fast oxidative fibers and fast glycolytic fibers.

17. A method for determining whether a subject will respond to a treatment for improving muscle function, which comprises:
measuring the activity of a focal adhesion signaling pathway member in a sample from a subject who has undergone or will undergo a treatment for muscle function that comprises (i) enhancing focal adhesion signaling in a muscle of a subject; and (ii) providing a load to the muscle; and
determining whether the subject will respond to the treatment based on the measured activity.

18. The method of claim 17, the focal adhesion signaling pathway member is selected from the group consisting of focal adhesion kinase (FAK), ribosomal S6 kinase, mammalian target of rapamycin (mTOR), myosin I heavy chain, myosin II heavy chain, tenascin-c, tenascin-w, tenascin-y, bombesin, reactive oxygen species, seven transmembrane receptor, integrin α7β1, integrin α7A, integrin α7B, vinculin, dystrophin, dystroglycans, sarcoglycan (α, β, γ, δ) dystrobrevin, dysferlin, ankyrin, plectin, α-B-crystallin, zyxin, desmin, synemin, paranemin, laminin α2β1γ1 (laminin 2), laminin α2β2γ1 (laminin 4) laminin 2/4, laminin 8/9, laminin 10/11, collagen IV, collagen VI, fibronectin, and eukaryotic translation initiation factor 4E binding protein I (eIF4E-BP1).

19. The method of claim 18, wherein the S6 kinase activity measures the amount of S6 kinase RNA.

20. The method of claim 18, wherein the S6 kinase activity measures the amount of S6 kinase protein.

21. The method of claim 18, wherein the S6 kinase activity measures the degree of S6 kinase phosphorylation.

22. The method of claim 18, wherein the S6 kinase activity measures the phosphotransfer activity of S6 kinase.


SEQUENCE LISTING (34 sequences)

SEQ ID NO: 1 - DNA, 3201 bp, Gallus gallus
atggagcagaagctgatctccgaggaggacctgggatccatggcagcagcttaccttgatccaaacttgaatcatacaccaagttcaagtgcaaagacgcacctcggtactgggatggagcgttccccgggggccatggagcgagtcctaaaggtttttcactactttgaaaacagcagcgagccaacgacgtgggccagcattatccggcatggagatgctactgatgttcgaggcataatacagaagattgtggactgtcacaaagtgaaaaatgtggcctgctatgggttgcgactcagtcatctgcagtctgaggaggttcactggctgcacctggacatgggggtatccaatgtgagagagaaatttgaactagcacatcctccagaagaatggaaatatgaactgagaattcggtacctgcccaaaggatttctaaaccagttcactgaggacaaaccaactttaaattttttctatcagcaggtgaaaaatgactatatgttagaaatagcagatcaagtggaccaggaaattgctttgaaactaggttgccttgaaatcaggagatcctacggagagatgagaggcaatgcattagagaagaaatccaactatgaagtgctagaaaaagatgtcggtttaagacgattttttccgaagagtttgctagattcagtgaaggccaaaacactacgaaaattaatccaacagacatttcgacaatttgccaacctcaacagagaagaaagtattttgaaattctttgagatcctctctccagtgtacagatttgacaaggaatgcttcaagtgtgcccttggttcaagctggattatttcagtggagctggcaattggcccagaggaaggaatcagctaccttacagacaagggtgcaaatccaactcacctggcagattttaatcaagtacaaactattcagtattcaaacagtgaagacaaggacagaaaagggatgttgcaactgaagatagctggtgcacctgagcctctgacagtgacagcaccatccttaaccattgcagagaatatggctgacttgatagacggatactgccgactggtgaatggagccacgcaatcttttattatcaggccacagaaagaaggtgaaagagctttaccatcaataccaaagctggccaacaatgagaagcaaggagtaaggtcgcacacagtctctgtatcagaaacagatgactatgcagagataatagatgaagaagatacttatacaatgccatcaaccagagattatgaaattcaaagggagagaattgaactggggcgctgcattggtgaaggacagtttggagatgtgcaccaaggaatttacatgagtccggaaaatccagctatggctgtagcaatcaaaacatgtaaaaactgcacctcagacagcgttagagaaaagttcctacaagaagccttaacaatgcgtcagtttgatcatcctcacattgtgaagctcattggagttattacagaaaacccagtgtggataatcatggagctctgtacacttggagagttgagatcgtttctgcaagtaagaaaattcagcttggacctggcctccctcatcctctacgcttaccagcttagcacagcacttgcttacctagagagcaaaagatttgtacatagagatattgctgctaggaacgtgctggtatctgccactgactgtgtgaaattgggtgactttggcttatcccgatacatggaagacagtacttactataaagcttccaaaggaaagttacctatcaaatggatggctccagagtcaatcaacttccgacggtttacctcagcaagcgatgtgtggatgtttggtgtgtgtatgtgggagatcctgatgcatggggtaaagcccttccagggagtgaaaaataatgatgttattggtcggattgagaacggtgagcggctccccatgcctccgaactgccctcccaccctctacagccttatgaccaagtgctgggcatacgaccctagtagacgacccaggtttactgaacttaaagcacaactcagtacaatactggaggaggagaagctgcagcaagaggaacgaatgagaatggaatccaggcgacaagtcacagtatcctgggactcaggaggatcagatgaagctcctcccaagcccagcaggcctggttaccccagcccaaggtccagtgaagggttttatccgagtcctcagcatatggtacagccaaatcactaccaggtatctggctactctggttctcatgggataccagccatggcaggcagcatttatcctgggcaagcttctctcttggatcaaacagattcctggaaccatcgacctcaggaagtatcagcatggcagccaaacatggaggattcgggcactttggatgtacgaggaatggggcaggttctgcccacacatctcatggaggagaggttaataagacaacagcaagagatggaagaagatcaacgctggcttgagaaagaggaacgattcctggtaatgaaacctgatgtgcggctctccagaggcagcattgaacgggaggacggaggtctccagggcccagctggtaaccagcacatatatcagcctgtgggtaaaccagatcatgccgctccaccaaagaagccccctcgccctggagccccccacttgggcagcctcgcgagcctgaacagccccgtggacagctacaacgaaggcgtgaagatcaagccacaggaaatcagccctcctcctacggccaacctggaccgctccaatgacaaagtctatgagaatgtaaccgggctggtgaaagctgtcatagagatgtccagtaaaatacagccagctccgccagaggagtacgtgcccatggtaaaggaggttggcttggcgctgagaaccttgctagcaacagtggatgagtcgctgccagtgcttcctgcaagcacccacagagagattgagatggcccagaaactgctgaactctgacctggctgagctcattaacaagatgaagctggcccagcagtacgtcatgaccagcctgcagcaggagtacaagaagcaaatgctgacggctgctcacgctctggctgtggatgccaagaacttgctggatgtcatcgatcaagccagactgaaaatgatcagccagtccaggccccactaa

SEQ ID NO: 2 - DNA, 5942 bp, Gallus gallus
ggggtttgacaggacggcgaggaatccgggagccgacagctggctgcagtacctctgcttcgtggaggctgcccgtggcaggatctgatccgtcagcccacacgagaataagcgtgccaagaaaggaaaggaaactcaacttagtttgaactggctctcaaatttctcctccagtctacaaaggccaaacaaatataagactccatcagctttgaaaaggaactgagcactacaatgggactcccttcccaggttttggcctgtgccatcttaggtttgctgtaccagcatgccagtggtgggctcatcaagcgaattatccggcagaagcgggagactgggctcaatgtgaccttaccagaggataatcagcctgtggttttcaatcatgtctacaacatcaagctgcctgttggctccctttgctctgtggacctggacacagcaagcggggacgcagacctgaaggcagaaattgagcctgtcaagaattacgaggagcatacggtgaatgaggggaaccagattgtcttcacgcaccgcatcaacattccccgccgggcctgtggctgtgcggctgccccagacatcaaggacctgctgagcagactggaggagctggaggggctggtatcctccctccgggagcagtgtgccagcggggctggatgctgtcctaattcccagacagcagaaggtcgcctggacacggccccctattgcagtgggcacggcaactacagcaccgagatctgtggctgcgtgtgcgagccaggctggaaaggccccaactgctccgaaccggcctgcccacgcaactgcctcaaccgcggcctctgcgtgcggggcaaatgcatctgcgaggagggctttaccggcgaggactgcagccaggctgcctgcccgtctgactgcaacgaccaaggcaagtgtgtggatggggtgtgcgtctgcttcgagggctacacgggcccggactgcggcgaggagctctgcccccacgggtgtggcattcacgggcgctgtgtgggtggacgctgtgtgtgccacgagggcttcactggcgaggactgcaatgagcccctgtgccccaacaactgtcacaaccgcgggcgctgtgtggacaacgagtgcgtctgcgatgagggctacacgggagaggactgcggcgagctgatttgccccaatgactgctttgaccgtgggcgctgtatcaacgggacctgcttctgcgaggagggctacactggagaggactgcggcgagctgacctgccccaacaactgcaacggcaacgggcgctgcgagaacgggctgtgtgtgtgccatgagggcttcgtgggggatgactgcagccagaagaggtgcccgaaggactgcaataaccgcgggcactgcgtggatgggcgctgtgtgtgccatgaggggtacctgggggaggactgtggggagctgcggtgccccaacgactgccacaaccgcgggcgctgcatcaatgggcagtgtgtgtgtgatgagggattcattggggaggactgtggagagctgcggtgccccaacgactgccacaaccgcgggcgctgcgtcaatgggcagtgcgagtgccacgagggattcatcggggaggactgcggggagctgcggtgtcccaacgactgcaacagccatgggcgctgtgtcaatgggcagtgcgtgtgtgatgaggggtacacaggggaggactgcggggagttgcggtgccccaacgactgccacaaccgcgggcgctgcgtggagggacgctgtgtgtgtgacaacggcttcatgggggaggactgcggggagctgtcctgtcccaatgactgccaccagcacgggcgctgcgtcgatgggcgctgcgtgtgccacgagggcttcactggggaagactgccgggaacggtcctgccccaatgactgcaacaacgtgggccgctgtgtcgagggacggtgtgtctgtgaggaaggttacatggggatcgactgttctgatgtgtctcctccaacggagctgactgtaacgaatgtaacagataaaacggtaaatctggaatggaagcatgagaatctcgtcaatgagtaccttgtcacctatgtccctaccagcagtggtggcttagatctacagttcaccgtaccaggaaaccagacatctgccactattcatgagctggagcctggtgtggaatacttcatccgtgtctttgcaatccttaaaaacaagaaaagtattccagtcagtgccagagtagcgacatatttgcctgctccagaaggtctgaaattcaaatctgttagagaaacgtctgtccaggtggaatgggatcctctgagcatttcctttgatggctgggagctggtctttcgtaatatgcagaaaaaggatgataatggagacataaccagcagcttgaaaaggccggagacatcatatatgcagccaggattggcaccaggacaacagtataatgtatcccttcatatagtgaaaaacaataccagaggaccagggctatcccgagtgataaccacaaaactcgatgcccctagccagattgaggcgaaagatgtcacagacaccacagctctgatcacatggtccaaacccttggctgaaattgaaggcatagagctcacatatggccccaaggatgttccaggggacaggactaccattgacctctctgaggatgaaaaccaatattctattggaaacctgaggccacacacagaatatgaagtgacactcatttctcggcgaggggacatggagagtgaccctgcaaaagaagtctttgtcacagacttggatgctccacgaaacctgaagcgagtgtcacagacagacaacagcattactttggagtggaagaacagccatgcaaatattgataattaccgaattaagtttgctcccatttctggtggagaccacactgagctgacagtgccaaagggcaaccaagcaacaaccagagctacactcacaggtttgagacctggaactgaatatggcattggagtgacagcagtgagacaggacagggaaagtgctcctgctaccattaatgctggcactgatcttgataaccccaaggacttggaagtcagtgaccccactgaaaccaccctgtcccttcgctggagaagaccagtggccaaatttgatcgttaccgcctcacttacgttagcccctctggaaagaagaacgaaatggagatccctgtggacagcacctcttttatcctgagaggattagacgcagggacggagtacaccatcagtctagtggcagagaaaggcagacacaaaagcaaacccacaaccatcaagggttcgactgaggaagaacctgagcttggaaacttatcagtgtcagagactggctgggatggtttccagctcacctggacagcagccgacggggcctatgagaactttgtcattcaggtgcagcagtctgacaatccagaagaaacctggaacattacagtccccggcggacagcactctgtgaacgttacaggcctcaaggccaacacaccttataacgtcacactttacggtgtgattcgaggctacagaaccaaacccctttatgttgaaaccacgacaggagcacaccccgaagttggtgagctaaccgtttccgacattactcctgaaagcttcaacctttcttggacgaccaccaacggggactttgacgcctttactattgaaattattgattctaacaggttgctggagcccatggagttcaacatctcaggcaattcaagaacagctcatatctcagggctttcccccagcactgattttattgtctacctctatgggatctctcatggtttccgcacacaggcaataagtgctgcggctacaacagaggcagaacccgaggtggacaaccttctggtttcagatgctaccccagacggcttccgtctgtcctggactgcagatgatggggttttcgacagttttgttctaaaaatcagggataccaaaaggaaatctgatccactggaactcatagtaccaggccatgagcgcacccatgatataacagggctgaaagagggcactgagtatgaaattgagctctatggagttagcagtggacggcgctcccaacccataaattcagtagcaaccacagttgtgggatctcccaagggaatctctttctcggacatcacagaaaactctgctacagtcagctggacacccccccgcagccgtgtggatagctacagggtctcctatgtccccatcacaggcggcactcccaatgttgttacagttgatggaagcaagacaaggacaaagctggtgaagttagtcccaggtgtagactacaacgttaatatcatctctgtgaaaggctttgaagaaagcgaacccatttctggaattctgaaaacagctctggacagcccgtcaggactggtagtgatgaacattacagactcggaggctctggcaacctggcagcctgcaattgcagctgtggataattacattgtctcctactcttctgaggatgagccagaagttacacagatggtatcaggaaacacagtggagtacgacctgaatggccttcgacctgcgacagagtacaccctgagggtgcatgcagtgaaggatgcgcagaagagcgagaccctctccacccagttcactacaggactcgatgctccaaaagatttaagtgctaccgaggttcagtcagaaacagctgtgataacgtggaggcctccacgtgctcctgtcactgattacctcctgacctacgagtccattgatggcagagtcaaggaagtcatcctagaccctgagacgacctcctacaccctgacagagctgagcccatccactcaatacacagtgaaacttcaggcactgagcagatctatgaggagcaaaatgatccagactgttttcaccacaactggtcttctttatccttatcctaaagactgctcccaagctctcctgaatggagaggtcacctctgggctctacactatttatctgaatggagacaggacacagcctctgcaagtcttctgtgacatggctgaagatggaggcggatggattgtgttcctgaggcgtcaaaatggaaaggaagatttctacaggaactggaagaattacgtggccggctttggagatcccaaggatgaattctggataggtctggagaacctccacaaaatcagctctcaggggcagtacgagctgcgtgtggatctgagagacagaggtgagacagcctatgctgtgtacgacaagttcagcgttggagatgccaagacccggtaccggctgagggtggatggctacagtggcacagcaggtgactccatgacctaccataatggaagatccttctccacttttgacaaggacaatgattctgctatcaccaactgtgctttgtcatacaagggtgctttctggtacaagaattgtcaccgagtcaatctgatgggcagatatggtgacaacaaccacagtcagggtgttaattggttccactggaagggccacgaatactccatccagtttgcagagatgaaactgagaccctccagctttcggaatctggaaggaagacgaaagcgagcataaagccttgggatggtgaaagggctacgggcagggcaacatggggagggacagagagcggggggcatgggaggatctctggcatcactggggttatgggtgtgaggagctggtagtcgtaccaaagcatcgcaacccttggcacaagagcccaaacaacgagccttacgtgtcccagcaattccacagagcagctccagctctgcccactgctgatgtccttcacgccaaagacaacgatctcaagggttgtatgctgttttcttcatttttcttttctcagc

SEQ ID NO: 3 - DNA, 4033 bp, Artificial Sequence
  Description of Artificial Sequence Synthetic polynucleotide
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagcttctagctagaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggccgcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagaccgtataacgtgctttcctcgttggaatcagagcgggagctaaacaggaggccgattaaagggattttagacaggaacggtacgccagctggattaccgcggtctttctcaacgtaacactttacagcggcgcgtcatttgatatgatgcgccccgcttcccgataagggagcaggccagtaaaagcattacccgtggtggggttcccgagcggccaaagggagcagactctaaatctgccgtcatcgacttcgaaggttcgaatccttcccccaccaccatcactttcaaaagtccgaaagaatctgctccctgcttgtgtgttggaggtcgctgagtagtgcgcgagtaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcatcccgccatggtatcaacgccatatttctatttacagtagggacctcttcgttgtgtaggtaccgctgtattcctagggaaatagtagaggcaccttgaactgtctgcatcagccatatagcccccgctgttcgacttacaaacacaggcacagtactgacaaacccatacacctcctctgaaatacccatagttgctagggctgtctccgaactcattacaccctccaaagtcagagctgtaatttcgccatcaagggcagcgagggcttctccagataaaatagcttctgccgagagtcccgtaagggtagacacttcagctaatccctcgatgaggtctactagaatagtcagtgcggctcccattttgaaaattcacttacttgatcagcttcagaagatggcggagggcctccaacacagtaattttcctcccgactcttaaaatagaaaatgtcaagtcagttaagcaggaagtggactaactgacgcagctggccgtgcgacatcctcttttaattagttgctaggcaacgccctccagagggcgtgtggttttgcaagaggaagcaaaagcctctccacccaggcctagaatgtttccacccaatcattactatgacaacagctgttttttttagtattaagcagaggccggggacccctgggcccgcttactctggagaaaaagaagagaggcattgtagaggcttccagaggcaacttgtcaaaacaggactgcttctatttctgtcacactgtctggccctgtcacaaggtccagcacctccataccccctttaataagcagtttgggaacgggtgcgggtcttactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaattc

SEQ ID NO: 4 - DNA, 10 bp, Artificial Sequence
  Description of Artificial Sequence Synthetic oligonucleotide
gccggcggag

SEQ ID NO: 5 - RNA, 10 nt, Artificial Sequence
  Description of Artificial Sequence Synthetic oligonucleotide
cucauaaggu

SEQ ID NO: 6 - RNA, 10 nt, Artificial Sequence
  Description of Artificial Sequence Synthetic oligonucleotide
gacuuugauu

SEQ ID NO: 7 - DNA, 10 bp, Artificial Sequence
  Description of Artificial Sequence Synthetic oligonucleotide
cggaacccaa

SEQ ID NO: 8 - RNA, 10 nt, Artificial Sequence
  Description of Artificial Sequence Synthetic oligonucleotide
auacuccccc

SEQ ID NO: 9 - RNA, 10 nt, Artificial Sequence
  Description of Artificial Sequence Synthetic oligonucleotide
ccuugcgacc

SEQ ID NO: 10 - protein, 9 aa, Artificial Sequence
  Description of Artificial Sequence Synthetic peptide
DYKDDDDKG

SEQ ID NO: 11 - protein, 6 aa, Artificial Sequence
  Description of Artificial Sequence Synthetic peptide
DTYRYI

SEQ ID NO: 12 - protein, 14 aa, Artificial Sequence
  Description of Artificial Sequence Synthetic peptide
GKPIPNPLLGLDST

SEQ ID NO: 13 - protein, 10 aa, Artificial Sequence
  Description of Artificial Sequence Synthetic peptide
EQKLISEEDL

SEQ ID NO: 14 - protein, 11 aa, Artificial Sequence
  Description of Artificial Sequence Synthetic peptide
QPELAPEDPED

SEQ ID NO: 15 - protein, 9 aa, Artificial Sequence
  Description of Artificial Sequence Synthetic peptide
YPYDVPDYA

SEQ ID NO: 16 - protein, 11 aa, Artificial Sequence
  Description of Artificial Sequence Synthetic peptide
YTDIEMNRLGK

SEQ ID NO: 17 - protein, 6 aa, Artificial Sequence
  Description of Artificial Sequence Synthetic 6xHis tag
HHHHHH

SEQ ID NO: 18 - protein, 7 aa, Artificial Sequence
  Description of Artificial Sequence Synthetic peptide
CCXXXCC

SEQ ID NO: 19 - protein, 6 aa, Artificial Sequence
  Description of Artificial Sequence Synthetic peptide
CCPGCC

SEQ ID NO: 20 - protein, 6 aa, Artificial Sequence
  Description of Artificial Sequence Synthetic peptide
LVPRGS

SEQ ID NO: 21 - protein, 5 aa, Artificial Sequence
  Description of Artificial Sequence Synthetic peptide
DDDDK

SEQ ID NO: 22 - protein, 7 aa, Artificial Sequence
  Description of Artificial Sequence Synthetic peptide
ENLYFQG

SEQ ID NO: 23 - protein, 8 aa, Artificial Sequence
  Description of Artificial Sequence Synthetic peptide
LEVLFQGP

SEQ ID NO: 24 - protein, 9 aa, Artificial Sequence
  Description of Artificial Sequence Synthetic peptide
RRRLRRLRA

SEQ ID NO: 25 - protein, 1066 aa, Gallus gallus
MEQKLISEEDLGSMAAAYLDPNLNHTPSSSAKTHLGTGMERSPGAMERVLKVFHYFENSSEPTTWASIIRHGDATDVRGIIQKIVDCHKVKNVACYGLRLSHLQSEEVHWLHLDMGVSNVREKFELAHPPEEWKYELRIRYLPKGFLNQFTEDKPTLNFFYQQVKNDYMLEIADQVDQEIALKLGCLEIRRSYGEMRGNALEKKSNYEVLEKDVGLRRFFPKSLLDSVKAKTLRKLIQQTFRQFANLNREESILKFFEILSPVYRFDKECFKCALGSSWIISVELAIGPEEGISYLTDKGANPTHLADFNQVQTIQYSNSEDKDRKGMLQLKIAGAPEPLTVTAPSLTIAENMADLIDGYCRLVNGATQSFIIRPQKEGERALPSIPKLANNEKQGVRSHTVSVSETDDYAEIIDEEDTYTMPSTRDYEIQRERIELGRCIGEGQFGDVHQGIYMSPENPAMAVAIKTCKNCTSDSVREKFLQEALTMRQFDHPHIVKLIGVITENPVWIIMELCTLGELRSFLQVRKFSLDLASLILYAYQLSTALAYLESKRFVHRDIAARNVLVSATDCVKLGDFGLSRYMEDSTYYKASKGKLPIKWMAPESINFRRFTSASDVWMFGVCMWEILMHGVKPFQGVKNNDVIGRIENGERLPMPPNCPPTLYSLMTKCWAYDPSRRPRFTELKAQLSTILEEEKLQQEERMRMESRRQVTVSWDSGGSDEAPPKPSRPGYPSPRSSEGFYPSPQHMVQPNHYQVSGYSGSHGIPAMAGSIYPGQASLLDQTDSWNHRPQEVSAWQPNMEDSGTLDVRGMGQVLPTHLMEERLIRQQQEMEEDQRWLEKEERFLVMKPDVRLSRGSIEREDGGLQGPAGNQHIYQPVGKPDHAAPPKKPPRPGAPHLGSLASLNSPVDSYNEGVKIKPQEISPPPTANLDRSNDKVYENVTGLVKAVIEMSSKIQPAPPEEYVPMVKEVGLALRTLLATVDESLPVLPASTHREIEMAQKLLNSDLAELINKMKLAQQYVMTSLQQEYKKQMLTAAHALAVDAKNLLDVIDQARLKMISQSRPH

SEQ ID NO: 26 - protein, 24 aa, Mus sp.
MGAVTWLLPGIFLALFALTPEGGV

SEQ ID NO: 27 - protein, 17 aa, Mus sp.
LREQCTMGTGCCLQPAE

SEQ ID NO: 28 - DNA, 50 bp, Mus sp.
ctgccaggcatctttctagctttgtttgccctcactcccgaaggtggggt

SEQ ID NO: 29 - DNA, 14 bp, Mus sp.
aagggagcggtgca

SEQ ID NO: 30 - DNA, 29 bp, Mus sp.
gtacaggctgttgcctccaacctgcagaa

SEQ ID NO: 31 - protein, 11 aa, Mus sp.
MGTGCCLQPAE

SEQ ID NO: 32 - DNA, 52 bp, Mus sp.
ctgctctttactgaaggctctttactattgctttatgataatgtttcatagt

SEQ ID NO: 33 - DNA, 15 bp, Mus sp.
agaagctggtcgagt

SEQ ID NO: 34 - DNA, 29 bp, Mus sp.
gtacaggctgttgcctccaacctgcagaa